SEQUENCE LISTING (52 sequences)

SEQ ID NO: 1 - protein, 30 aa, Coccidioides immitis
  source 1..30: note = RMSCC 2394
ATFSNFGSVLDIFAPGVDVESTMPGGGTQM

SEQ ID NO: 2 - protein, 30 aa, Coccidioides immitis
PTATLPPSPTGSACEPHGDHWHCEPLPTST

SEQ ID NO: 3 - protein, 30 aa, Coccidioides immitis
TGTCEPHGDHWHCPPGIPEPTTPPPSEPSI

SEQ ID NO: 4 - protein, 30 aa, Coccidioides immitis
PPPSEPSIPATTGTCEPHGDHWHCPPGVPE

SEQ ID NO: 5 - protein, 30 aa, Coccidioides posadasii
  source 1..30: note = str. Silveira
KILEKPLAKSTGAKGKNKKKKKKPAKKQAE

SEQ ID NO: 6 - protein, 30 aa, Coccidioides immitis
  source 1..30: note = H538.4
ASPHHQPPVDKPKGGQEFYEAPKPEEPATT

SEQ ID NO: 7 - protein, 30 aa, Coccidioides posadasii
  source 1..30: note = RMSCC 3488
QRDYPTIDWEGLEDRIQAYFAELDQILTRK

SEQ ID NO: 8 - protein, 30 aa, Coccidioides immitis
PKNPNPQPNPYDPHQPKRKPKPEPKPEPYD

SEQ ID NO: 9 - protein, 30 aa, Coccidioides immitis
EPKPEPYDPYWPKPNPYDPHQPKRKPKPEP

SEQ ID NO: 10 - protein, 30 aa, Coccidioides immitis
KRKPKPEPKPEPYDPYWPKPKPFDPFRPKP

SEQ ID NO: 11 - protein, 30 aa, Coccidioides immitis
FDPFRPKPKPYDPFRPESKPFDPFRPKPKP

SEQ ID NO: 12 - protein, 30 aa, Coccidioides immitis
RPKPKPYDPFRPEPKPYDPFRPESKPFDPF

SEQ ID NO: 13 - protein, 30 aa, Coccidioides immitis
ESKPFDPFRPESKPYDPFRPESKPYDPYWP

SEQ ID NO: 14 - protein, 30 aa, Coccidioides immitis
PYWPKPKPFDPYWPESKPKPKPKPRDPHQE

SEQ ID NO: 15 - protein, 30 aa, Coccidioides immitis
KPRDPHQETPQDPYQPKPQDPEPRPGDGDR

SEQ ID NO: 16 - protein, 30 aa, Coccidioides immitis
PRPGDGDRVINVGFPAGGLICHPACFPPSH

SEQ ID NO: 17 - protein, 30 aa, Coccidioides immitis
STCCELPKPKRESNPHDSDQPNPRPGDHDG

SEQ ID NO: 18 - protein, 30 aa, Coccidioides immitis
HPHHHPHDSHQPNPRPGDRDGPLDPNQPAP

SEQ ID NO: 19 - protein, 30 aa, Coccidioides posadasii
  source 1..30: note = str. Silveira
RLLDDYDAHRADRSSGALSFAPKFDVRESK

SEQ ID NO: 20 - protein, 30 aa, Coccidioides posadasii
  source 1..30: note = str. Silveira
YTSGTPPGKASEAAKGKETETGERYWVSER

SEQ ID NO: 21 - protein, 30 aa, Coccidioides posadasii
  source 1..30: note = RMSCC 2394
VKEKEESVQQDTSKTGMEADNELDGSAIDG

SEQ ID NO: 22 - protein, 30 aa, Coccidioides immitis
  source 1..30: note = RMSCC 2394
ESPRGWWPDLQPKNPNPQPKPYDPYQPKPK

SEQ ID NO: 23 - protein, 30 aa, Coccidioides immitis
  source 1..30: note = RMSCC 2394
PESKPYDPYQPELKPFDPYQPESKPFDPYW

SEQ ID NO: 24 - protein, 30 aa, Coccidioides immitis
  source 1..30: note = RMSCC 2394
SKPFDPYWPESKPFDPYQPESKPFDPYQPE

SEQ ID NO: 25 - protein, 30 aa, Coccidioides immitis
  source 1..30: note = RMSCC 2394
PFDPYQPESKPFDPYWPESNHYDSYQLEPR

SEQ ID NO: 26 - protein, 30 aa, Coccidioides immitis
  source 1..30: note = RMSCC 2394
DSYQLEPRAGPRPFLRIPRPRPRPHRPRPH

SEQ ID NO: 27 - protein, 30 aa, Coccidioides immitis
  source 1..30: note = RMSCC 2394
GPHRPSRPQPRPQPVPIPQNPHLPNLDASL

SEQ ID NO: 28 - protein, 30 aa, Coccidioides immitis
  source 1..30: note = RMSCC 2394
LPNLDASLIDALLPKPHQETPQDPYQPKPQ

SEQ ID NO: 29 - protein, 30 aa, Coccidioides immitis
  source 1..30: note = RMSCC 2394
DPYQPKPQDPEPRPGDGDRVINVGFPAGDL

SEQ ID NO: 30 - protein, 30 aa, Coccidioides immitis
  source 1..30: note = RMSCC 2394
DLDCFTCCKPLKPKPKPESNPHDSDQPNPR

SEQ ID NO: 31 - protein, 30 aa, Coccidioides immitis
  source 1..30: note = RMSCC 2394
DSDQPNPRPGDHDGPIIDHGALDPNHPHHH

SEQ ID NO: 32 - protein, 30 aa, Coccidioides immitis
GDHDGPIIDHGALDPNHPHHHPHDSHQPAP

SEQ ID NO: 33 - protein, 30 aa, Coccidioides posadasii
  source 1..30: note = RMSCC 3488
KPKPYDPHQPKPKPKPNPTSLIPSRLIRSI

SEQ ID NO: 34 - protein, 30 aa, Coccidioides posadasii
  source 1..30: note = RMSCC 3488
PRPFLRIPRPRPSPHRPRPHRPSRPSRPRP

SEQ ID NO: 35 - protein, 30 aa, Coccidioides immitis
  source 1..30: note = RMSCC 2394
PTTSTTASQPTNGPPADECEPHGDHWHCPP

SEQ ID NO: 36 - protein, 30 aa, Coccidioides immitis
ASEFVTMAPRITLSDFPLLPLTYDDLNSMV

SEQ ID NO: 37 - protein, 30 aa, Coccidioides immitis
VTAGVPHLQQAGVNTPNLHPTTVGVLNELM

SEQ ID NO: 38 - protein, 30 aa, Coccidioides posadasii
  source 1..30: note = str. Silveira
EHSYCDTYGCDGPMEPKTPKPTDCYGDCED

SEQ ID NO: 39 - protein, 30 aa, Coccidioides posadasii
  source 1..30: note = str. Silveira
PPKKYGDCDDYDGYCDGPSKTSIKPEPPKP

SEQ ID NO: 40 - protein, 30 aa, Coccidioides posadasii
  source 1..30: note = str. Silveira
MALSPTASCALACPEGDVCCQAACVGVPCP

SEQ ID NO: 41 - protein, 30 aa, Coccidioides posadasii
  source 1..30: note = str. Silveira
CDQGNGSPAAIEAYAKCQSDCIKTYFFTGS

SEQ ID NO: 42 - protein, 30 aa, Coccidioides immitis
SDEEEEAEAEADKELSAKLSPNAADGKVEL

SEQ ID NO: 43 - protein, 30 aa, Coccidioides immitis
ATPSHYDEYGYKMRKRGAKEHSYCDTYGCD

SEQ ID NO: 44 - protein, 30 aa, Coccidioides immitis
YCDTYGCDGPMEPKPPKPTDCYGDCEDGYD

SEQ ID NO: 45 - protein, 30 aa, Coccidioides posadasii
  source 1..30: note = RMSCC 3488
NEAYEAGLIGKNASGSGYDFDIYIHRGMGA

SEQ ID NO: 46 - protein, 30 aa, Coccidioides immitis
KLENVPSPDFGLFSDILIGTIRSEIADSSE

SEQ ID NO: 47 - protein, 30 aa, Coccidioides immitis
SIAENAIKAEIMAKKDDDKHQHQGAKDFLV

SEQ ID NO: 48 - protein, 30 aa, Coccidioides posadasii
  source 1..30: note = str. Silveira
QSYDGGFAQAPWLEAHAGLTYSALGTLSFL

SEQ ID NO: 49 - protein, 30 aa, Coccidioides immitis
YPPHDCQSYGDDYGNCKGEKPSATPSHYDE

SEQ ID NO: 50 - protein, 30 aa, Coccidioides immitis
VDATEKFLIETAPGETRWVTEAEKFELKEK

SEQ ID NO: 51 - protein, 8 aa, Coccidioides sp.
YCDTYGCD

SEQ ID NO: 52 - protein, 9 aa, Coccidioides sp.
PESKPFDPY

(19) United States
(12) Patent Application Publication
(10) Pub. No.: US 2026/0309707 A1
(43) Pub. Date: Oct. 8, 2026

(54) COMPOSITIONS AND METHODS FOR DETECTION AND TREATMENT OF COCCIDIOIDES INFECTION

(71) Applicant: The Translational Genomics Research Institute, Phoenix, AZ (US)

(72) Inventors: Heather Lynn Mead, Flagstaff, AZ (US); John Altin, Flagstaff, AZ (US); Jason Ladner, Flagstaff, AZ (US); Evan Elko, Flagstaff, AZ (US)

(21) Appl. No.: 19/640,343

(22) Filed: Apr. 6, 2026

Related U.S. Application Data
(60) Provisional application No. 63/783,996, filed on Apr. 5, 2025.

(51) Int. Cl. G01N 33/569; A61K 39/00; G01N 33/543

(52) U.S. Cl. CPC G01N 33/56961; A61K 39/0002; G01N 33/54386; G01N 2333/37; G01N 2469/20

(57) ABSTRACT

The present disclosure provides methods and compositions that may be used to detect and the presence of Coccidioides in a sample. By designing a proteome-wide peptide library covering covers both species of Coccidioides, new epitopes were found having antibody reactivity. A multi-peptide assay was used to validate the diagnostic utility of the discovered antigens. Thus, the disclosed technology reveals new Coccidioides targets for the development of diagnostics, vaccines and therapeutics.

### RELATED APPLICATIONS

This application claims the benefit of U.S. Provisional Patent Application No. 63/783,996, filed Apr. 5, 2025, titled “COMPOSITIONS AND METHODS FOR DETECTION AND TREATMENT OF COCCIDIOIDES INFECTION,” the entirety of the disclosure of which is hereby incorporated by this reference.

### SEQUENCE LISTING

In accordance with 37 C.F.R. § 1.831, the present specification makes reference to a Sequence Listing submitted electronically in the form of an XML file (entitled “274US-PAT”, created on Apr. 6, 2026, 51,711 bytes in size). The entire contents of the Sequence Listing are herein incorporated by reference in their entirety, with the intention that, upon publication (including issuance), this incorporated Sequence Listing will be inserted in the published document immediately before the claims.

### FEDERAL FUNDING STATEMENT

This invention was made with government support under AI180987 and AI166058 awarded by the National Institute of Health. The government has certain rights in the invention.

### TECHNICAL FIELD

The present invention is related to compositions and methods for diagnosing, treating, and preventing disease and predicting immune response to disease, and more particularly, is related to compositions and methods for diagnosing, treatment, prevention, and prognosis of Coccidioides infections.

### BACKGROUND

Coccidioidomycosis (CM), commonly referred to as Valley Fever (VF), is an environmentally acquired fungal disease endemic in the southwestern United States, where its incidence has been increasing in recent years¹. The disease has a broad range of clinical manifestations, ranging from mild and self-limiting infections to chronic pneumonia often accompanied by flu-like symptoms¹. In rare cases, the infection can become extrapulmonary and spread to other organs^{2,3}. Due to the presentation of symptoms similar to those of many bacterial infections, delays in diagnosis are common, often resulting in inappropriate use of antibiotics and increased risk of developing severe disease caused by the delay in correct evaluation and treatment^{3,4}. Even in endemic areas where awareness is high, a correct medical diagnosis typically requires multiple clinical visits and takes an average of 23 days⁴. The current diagnostic process for CM is imprecise, and clinicians often use multiple different approaches to balance turnaround time, accessibility and interpretability of the current diagnostic options^{2,5}.

Clinicians frequently use serology-based assays to aid in diagnosis of CM, as they are safer and more rapid than culture or microscopy based methods². Current serological diagnostics use either crude fungal lysate, or one of three known diagnostic antigens: Beta-glucosidase 2 (formerly referred to as tube precipitin, TP)⁶, complement fixation chitinase (CTS1, CF)⁷ and galactomannan⁸. These targets have been used in several different formats, including enzyme immunoassay (EIA)^{9,10}, lateral flow¹¹, immunodiffusion, and complement fixation¹², where they detect either IgM or IgG reactivity^{5,9}. The sensitivity of these assays range from ~30-87%, with specificity ranging from ~68-99%,¹³⁻¹⁶. Considerable effort has been put into increasing the performance of these assays and identifying new antigenic targets, however, identifying and testing potential antigens has been challenging due to the large number of potential immunogenic proteins. Coccidioides spp. are genetically and morphologically complex, with ~8,000 protein coding genes per strain, 50% of which are unannotated some of which are critical host specific morphology which evolved to evade the host immune system^{17,18}. Identification and production of unannotated proteins with immunogenic potential is labor intensive (crude lysate, SDS page, sequencing) and cost prohibitive (protein expression and purification at scale). Historically, the number of antigens (once identified) that could be tested was limited by the cost and sample volume requirements associated with establishing and running single-plex serology assays, such as enzyme-linked immunosorbent assays (ELISAs)¹⁹. However, recent advances in highly-multiplexed serology now allow for the assessment of antibody binding to hundreds of thousands of antigens in a single assay with minimal upfront investment and sample consumption^{20,21}.

Citation or identification of any document in this application is not an admission that such a document is available as prior art to the present invention.

### SUMMARY

A need exists for methods of diagnosing, treating, preventing, and determining an immune response to Coccidioides infections, including Coccidioidomycosis caused by Coccidioides infection. A further need exists to identify immunogenic targets with better diagnostic potential for the disease Coccidioidomycosis.

Coccidioidomycosis (CM), caused by Coccidioides spp., accounts for a significant proportion of community-acquired pneumonia in endemic regions such as the southwestern United States. However, CM remains underdiagnosed, in part because of the limitations of existing diagnostic assays. Coccidioides (also referred to herein as Cocci) has a complex life cycle and large proteome (approximately 7,000 to 9,000 proteins), and therefore a breadth of potential antigenic proteins (FIG. 1). The enormous diversity of possible Coccidioides specific antigens poses technical challenges in the identification of new targets recognized by patients' antibodies. There are likely uncharacterized antigens with diagnostic potential in the Coccidioides spp. proteome, however identifying these antigenic proteins from the large and poorly-understood proteomic space poses a significant challenge. Using a highly-multiplexed serology platform that measures antibody reactivity against a broad range of fully-customizable DNA-barcoded peptide antigens (PepSeq), we have designed the first proteome-wide peptide library, which covers both species of Coccidioides (FIG. 2). We developed a 244,000-plex 30mer library providing proteome-wide representation of Coccidioides spp. We utilized this library to characterize protein and epitope-level antibody reactivity in serum from CM patients, naturally infected pigtail macaques (PTMs), and healthy human and animal controls (from outside the endemic region). Our analysis revealed a panel of previously unidentified protein antigens, many containing multiple epitopes that were frequently recognized by IgG antibodies in CM patients. We observed 48 epitopes from six proteins that were uniquely recognized by the disease cohort. A subset of these epitopes were also commonly targeted by IgG in PTMs with CM. We adapted several of these targets into a pooled peptide ELISA that discriminated cases from controls in an independent validation cohort with high accuracy (AUC 0.95). An assay containing 16 of the epitopes was able to differentiate between Coccidioidomycosis patients and healthy controls (AUC 0.94). Together, these results demonstrate the power of peptide-based highly-multiplexed serology to discover novel fungal antigens and successfully identify CM specific epitope-resolved targets for new diagnostic assays.

The present disclosure includes methods for diagnosing, prognosing or monitoring the treatment of a Coccidioides infection in a subject by detecting in a sample from the subject the presence or absence of an antibody to a peptide comprising an amino acid sequence selected from the group consisting of SEQ ID NOS: 1-50.

In some aspects, the present disclosure relates to a method of detecting in a sample the presence of an antibody that binds to Coccidioides protein. The method may comprise the steps of providing a biological sample from a subject suspected to be infected with Coccidioides; contacting the biological sample with a peptide comprising an amino acid sequence having at least 85% sequence identity to an amino acid sequence selected from the group consisting of SEQ ID NOS: 1-50; and detecting antigen-antibody complexes formed.

In one aspect, the method comprises contacting the biological sample with a peptide comprising an amino acid sequence having at least 85%, at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOS: 1-50.

In various embodiments, methods for diagnosing, prognosing or monitoring the treatment of a Coccidioides infection in a subject may comprise the step of detecting in a sample from the subject the presence or absence of an antibody to a peptide comprising an amino acid sequence selected from the group consisting of SEQ ID NOS: 1-50.

Further, methods of serologically detecting an antibody to Coccidioides in a sample may comprise the steps of contacting the sample with a peptide under conditions sufficient to allow the binding of the antibody to the peptide. The peptide may comprise an amino acid sequence selected from the group consisting of SEQ ID NOS: 1-50 and detecting formation of an antibody-peptide complex comprising the antibody and the peptide. The formation of the antibody-peptide complex is indicative of an antibody to an epitope of a Coccidioides antigen being present in the sample.

The present disclosure further includes a peptide reactive with an antibody specific to Coccidioides. The peptide may comprise an amino acid sequence selected from the group consisting of SEQ ID NOS: 1-50. In some embodiments, a synthesized peptide is provided comprising an amino acid sequence selected from the group consisting of SEQ ID NOS: 1, 3, 7, 13, 15, 17, 24, 26, 29, 32, 36, 38, 39, 40, 43, and 44. In some embodiments, a synthesized peptide is provided comprising an amino acid sequence selected from the group consisting of SEQ ID NOS: 1-50 or an amino acid sequence having at least 85%, at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOS: 1-50.

According to further embodiments, an immunogenic composition is provided. The immunogenic composition may comprise a peptide with an amino acid sequence selected from the group consisting of SEQ ID NOS: 1-50. The immunogenic composition may comprise a peptide comprising an amino acid sequence selected from the group consisting of SEQ ID NOS: 1, 3, 7, 13, 15, 17, 24, 26, 29, 32, 36, 38, 39, 40, 43, and 44. The immunogenic composition may further comprise a pharmaceutically acceptable carrier or adjuvant. Thus, a method of recruiting an antifungal antibody in a subject to prevent or limit a Coccidioides infection in the subject is provided herein, wherein the antifungal antibody is specific to Coccidioides. A method of eliciting an immune response in a subject may comprise the step of administering to the subject an immunogenic composition including a peptide comprising an amino acid sequence selected from the group consisting of SEQ ID NOS: 1-50. The immunogenic composition may further comprise a pharmaceutically acceptable carrier or adjuvant. Thus, a method of recruiting an antifungal antibody in a subject to prevent or limit a Coccidioides infection in the subject is provided herein, wherein the antifungal antibody is specific to Coccidioides.

In certain aspects, the present disclosure provides a method of detecting in a sample the presence of an antibody that binds to a Coccidioides protein. The method may comprise the steps of providing a biological sample from a subject suspected to be infected with Coccidioides; contacting the biological sample with a peptide comprising an amino acid sequence selected from the group consisting of SEQ ID NOS: 1-50; and detecting antigen-antibody complexes formed. The method of detecting antigen-antibody complexes may comprise a technique selected from the group consisting of flow cytometry, immunohistochemistry, enzyme-linked immunosorbent assay (ELISA), Western Blot, and immunoaffinity chromatography.

In another aspect, the present invention provides for a method of detecting in a sample the presence of an antibody that binds to a Coccidioides protein, the method comprising: providing a biological sample from a subject suspected to be infected with Coccidioides; contacting the biological sample with a peptide comprising an amino acid sequence having at least 85% sequence identity to an amino acid sequence selected from the group consisting of SEQ ID NO: 1-50; and detecting antigen-antibody complexes formed. In certain embodiments, the peptide comprises an amino acid sequence having at least 85% sequence identity to an amino acid sequence selected from the group consisting of SEQ ID NO: 2-4, 8-9, 11-18, 23-25, 27, 29, 30-33, 35, 37-41, 43-44, and 48. In certain embodiments, the peptide comprises an amino acid sequence having at least 85% sequence identity to an amino acid sequence selected from the group consisting of SEQ ID NO: 1, 3, 7, 13, 15, 17, 24, 26, 29, 32, 36, 38, 39, 40, 43, and 44.

In certain embodiments, the method further comprises detecting in the sample the presence of one more additional antibodies that each binds to a Coccidioides protein, wherein the sample is contacted with one or more additional peptides comprising an amino acid sequence having at least 85% sequence identity to an amino acid sequence selected from the group consisting of SEQ ID NO: 1-50. In certain embodiments, the one or more additional peptides comprise an amino acid sequence having at least 85% sequence identity to an amino acid sequence selected from the group consisting of SEQ ID NO: 2-4, 8-9, 11-18, 23-25, 27, 29, 30-33, 35, 37-41, 43-44, and 48. In certain embodiments, the one or more additional peptides comprise an amino acid sequence having at least 85% sequence identity to an amino acid sequence selected from the group consisting of SEQ ID NO: 1, 3, 7, 13, 15, 17, 24, 26, 29, 32, 36, 38, 39, 40, 43, and 44. In certain embodiments, the method comprises detecting in the sample the presence of a first, second, third, fourth, fifth, and/or sixth antibody that binds to a Coccidioides protein, wherein the first peptide comprises an amino acid sequence having at least 85% sequence identity to an amino acid sequence selected from the group consisting of SEQ ID NO: 38, 39, 43, and 44, wherein the second peptide comprises an amino acid sequence having at least 85% sequence identity to an amino acid sequence selected from the group consisting of SEQ ID NO: 8-9, 11-18, 23-25, 27, and 29-33, wherein the third peptide comprises an amino acid sequence having at least 85% sequence identity to an amino acid sequence selected from the group consisting of SEQ ID NO: 2-4 and 35, wherein the fourth peptide comprises an amino acid sequence having at least 85% sequence identity to an amino acid sequence selected from the group consisting of SEQ ID NO: 40-41, wherein the fifth peptide comprises an amino acid sequence having at least 85% sequence identity to an amino acid sequence consisting of SEQ ID NO: 48, and/or wherein the sixth peptide comprises an amino acid sequence having at least 85% sequence identity to an amino acid sequence consisting of SEQ ID NO: 37. Thus, in embodiments, any combination of peptides selected from any of the proteins can be detected. For example, any number of peptides derived from a first protein and any number of peptides derived from any of the other proteins can be used for detection.

In certain embodiments, the biological sample is whole blood, serum, or plasma. In certain embodiments, the antibody is IgG. In certain embodiments, the antibody is IgA. In certain embodiments, the antibody is IgM. In certain embodiments, the subject is a human. In certain embodiments, the method of detecting antigen-antibody complexes comprises a technique selected from the group consisting of enzyme-linked immunosorbent assay (ELISA), western blot, flow cytometry, and immunoaffinity chromatography.

In certain embodiments, detection of the antigen-antibody complex is indicative of a Coccidioides infection. In certain embodiments, the method further comprises treating the subject for a Coccidioides infection if an antigen-antibody complex is detected.

In another aspect, the present invention provides for an immunogenic composition, comprising: one or more peptides comprising an amino acid sequence having at least 85% sequence identity to an amino acid sequence selected from the group consisting of SEQ ID NO: 1-50; and a pharmaceutically acceptable carrier or adjuvant. In certain embodiments, the amino acid sequence comprises 85% sequence identity to an amino acid sequence selected from the group consisting of SEQ ID NO: 2-4, 8-9, 11-18, 23-25, 27, 29, 30-33, 35, 37-41, 43-44, and 48. In certain embodiments, the amino acid sequence comprises 85% sequence identity to an amino acid sequence selected from the group consisting of SEQ ID NO: 1, 3, 7, 13, 15, 17, 24, 26, 29, 32, 36, 38, 39, 40, 43, and 44. In certain embodiments, the composition comprises a first, second, third, fourth, fifth, and/or sixth peptide, wherein the first peptide comprises an amino acid sequence having at least 85% sequence identity to an amino acid sequence selected from the group consisting of SEQ ID NO: 38, 39, 43, and 44, wherein the second peptide comprises an amino acid sequence having at least 85% sequence identity to an amino acid sequence selected from the group consisting of SEQ ID NO: 8-9, 11-18, 23-25, 27, and 29-33, wherein the third peptide comprises an amino acid sequence having at least 85% sequence identity to an amino acid sequence selected from the group consisting of SEQ ID NO: 2-4 and 35, wherein the fourth peptide comprises an amino acid sequence having at least 85% sequence identity to an amino acid sequence selected from the group consisting of SEQ ID NO: 40-41, wherein the fifth peptide comprises an amino acid sequence having at least 85% sequence identity to an amino acid sequence consisting of SEQ ID NO: 48, and/or wherein the sixth peptide comprises an amino acid sequence having at least 85% sequence identity to an amino acid sequence consisting of SEQ ID NO: 37.

In another aspect, the present invention provides for a method of eliciting an immune response in a subject, the method comprising administering to the subject the immunogenic composition of any embodiment herein.

In another aspect, the present invention provides for a kit for detecting the presence of Coccidioides in a subject comprising one or more peptides comprising an amino acid sequence having at least 85% sequence identity to an amino acid sequence selected from the group consisting of SEQ ID NOS: 1-50.

In certain embodiments, the one or more peptides comprise an amino acid sequence having at least 85% sequence identity to an amino acid sequence selected from the group consisting of SEQ ID NO: 2-4, 8-9, 11-18, 23-25, 27, 29, 30-33, 35, 37-41, 43-44, and 48. In certain embodiments, the kit comprises a first, second, third, fourth, fifth, and/or sixth peptide, wherein the first peptide comprises an amino acid sequence having at least 85% sequence identity to an amino acid sequence selected from the group consisting of SEQ ID NO: 38, 39, 43, and 44, wherein the second peptide comprises an amino acid sequence having at least 85% sequence identity to an amino acid sequence selected from the group consisting of SEQ ID NO: 8-9, 11-18, 23-25, 27, and 29-33, wherein the third peptide comprises an amino acid sequence having at least 85% sequence identity to an amino acid sequence selected from the group consisting of SEQ ID NO: 2-4 and 35, wherein the fourth peptide comprises an amino acid sequence having at least 85% sequence identity to an amino acid sequence selected from the group consisting of SEQ ID NO: 40-41, wherein the fifth peptide comprises an amino acid sequence having at least 85% sequence identity to an amino acid sequence consisting of SEQ ID NO: 48, and/or wherein the sixth peptide comprises an amino acid sequence having at least 85% sequence identity to an amino acid sequence consisting of SEQ ID NO: 37.

In certain embodiments, the one or more peptides comprise an amino acid sequence having at least 85% sequence identity to an amino acid sequence selected from the group consisting of SEQ ID NO: 1, 3, 7, 13, 15, 17, 24, 26, 29, 32, 36, 38, 39, 40, 43, and 44. In certain embodiments, the kit comprises 16 peptides comprising an amino acid sequence having at least 85% sequence identity to an amino acid sequence as set forth by SEQ ID NO: 1, 3, 7, 13, 15, 17, 24, 26, 29, 32, 36, 38, 39, 40, 43, and 44.

In certain embodiments, each peptide is biotinylated. In certain embodiments, the kit comprises a plate having microwells coated with the peptides.

The foregoing features and elements may be combined in various combinations without exclusivity, unless expressly indicated otherwise. These features and elements as well as the operation thereof will become more apparent in light of the following description. It should be understood, however, the following description is intended to be exemplary in nature and non-limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Implementations will hereinafter be described in conjunction with the appended and/or included DRAWINGS, where like designations denote like elements, and:

FIG. 1—illustrates binding of naïve B to antigens results in the generation of pathogen specific antibodies.

FIG. 2A-2C—Overview of a multiplexed peptide assay (“PepSeq”) protocol. FIG. 2A shows the design of a peptide library begins with a set of target protein sequences which are informatically converted into DNA encodings and constant regions (black segments) are added to each end. The corresponding DNA oligonucleotide library is prepared by massively-parallel DNA synthesis and converted in bulk to a corresponding library of DNA-barcoded peptides (‘PepSeq probes’) using in vitro transcription and translation taking advantage of intramolecular coupling mediated by a tethered puromycin (P) containing molecule. FIG. 2B shows a library of PepSeq probes are then incubated with antibodies to bind their cognate epitopes. The antibodies are then captured onto protein-bound beads via their constant regions, unbound probes are washed away, and bound probes are eluted. FIG. 2C shows the DNA tags of eluted PepSeq probes are amplified by PCR and the relative abundance of each probe is quantified using high-throughput sequencing. Changes in relative abundance provide a semi-quantitative measure of cognate antibody abundance/affinity for each peptide in the design.

FIG. 3A-3E—PepSeq identifies Coccidioides spp. peptides and proteins commonly recognized by antibodies in infected individuals. A PepSeq library (CWP) covering the full proteome of Coccidioides spp. was used to broadly assess anti-Coccidioides spp. antibody reactivity in the discovery cohort of 70 CM (Coccidioidomycosis) patient sera and 60 control sera from a non-endemic region in the US. FIG. 3A) A volcano plot showing the Benjamini-Hochberg corrected p-values from a Fisher's exact test comparing the number of positive and negative CM and non-endemic control samples (y-axis) versus OddsRatio (x-axis) for a subset of 884 peptides recognized by serum IgG antibodies in ≥5 serum samples. Significant peptides after multiple test correction are highlighted in red, non-significant peptides are shown in grey. Horizontal dashed line indicates an corrected p-value of 0.05. Vertical dashed line indicates an odds ratio of 1. FIG. 3B) Boxplots showing the Z score distributions for the 31 significantly enriched IgG peptides in the non-endemic control (blue) and CM (orange) cohorts. Red lines indicate the threshold set for each peptide (three standard deviations above the mean of the non-endemic control cohort). Colored bars at bottom indicate the ID of the protein from which each peptide was designed (see, FIG. 3C). A volcano plot showing the −log 10(P-value) from Fisher's exact test versus log 10 (OddsRatio) for a subset of 273 peptides recognized by serum IgM antibodies in ≥5 serum samples. Horizontal dashed line indicates a Benjamini-Hochberg corrected P-value of 0.05. Vertical dashed line indicates an odds ratio of 1. FIG. 3D) A volcano plot showing the −log 10(P-value) from a Fisher's exact test versus log 10 (OddsRatio) for a subset of 32 peptides recognized by serum IgA antibodies in ≥5 serum samples. Horizontal dashed line indicates a Benjamini-Hochberg corrected P-value of 0.05. Vertical dashed line indicates an odds ratio of 1. FIG. 3E) Boxplots showing the Z score distribution of the two significantly enriched IgA peptides in the non-endemic control and CM cohorts.

FIG. 4A-4B—Epitope level reactivity profiles for the six significantly enriched human IgG proteins. FIG. 4A) Heatmap showing the proportion of positive samples in both CM and non-endemic control samples across the full protein sequence for the six proteins that contain significantly enriched peptides. Black bars in “Sig. pep” row indicates the location of the 31 significantly enriched peptides identified in FIG. 1. Asterisks in IgG antigen 3 indicate the two peptides of interest also detected in the PTM IgG screen. FIG. 4B) Heatmap showing the Z scores for reactive peptides across the IgG antigen 1 protein sequence for all samples that contained at least one reactive peptide.

FIG. 5A-5B—PepSeq identifies candidate diagnostic peptides with immunoreactivity in pigtail macaque serum. A PepSeq library (CWP) covering the full proteome of Coccidioides spp. was used to broadly assess anti-Coccidioides spp. antibody reactivity in the serum from 33 PTMs with confirmed natural Coccidioides spp. infections and 17 control sera from PTMs raised outside of the endemic region of the US. FIG. 5A) A volcano plot showing the Benjamini-Hochberg corrected p-values from a Fisher's exact test comparing the number of positive and negative CM and non-endemic control samples (y-axis) versus OddsRatio (x-axis) for a subset of 104 peptides recognized by PTM serum IgG antibodies in ≥5 serum samples. Significant peptides after multiple test correction are shown in red, non-significant peptides are shown in grey. Horizontal dashed line indicates an corrected p-value of 0.05. Vertical dashed line indicates an odds ratio of 1. FIG. 5B) Boxplots showing the Z score distribution of the two significantly enriched IgG peptides in the PTM raised outside of the endemic region (blue) and naturally infected CM PTM (orange) cohorts.

FIG. 6A-6D—Peptides identified by PepSeq effectively discriminate between non-endemic controls and CM patients in the discovery cohort, as well as an independent CM cohort. FIG. 6A, FIG. 6B) ROC curves for discriminating between CM and control samples in the discovery cohort using PepSeq Z scores. FIG. 6A) ROC curves for the 31 significantly enriched human peptides identified in the IgG screen (grey lines) as well as for the sum of Z scores for a subset of these peptides selected by forward (n=4, pink) and backward (n=9, brown) selection algorithms. AUCs indicated in lower right corner. FIG. 6B) ROC curves of PepSeq Z scores for the 16 peptides selected to be part of the peptide ELISA (grey) and the sum of the selected peptides (red). FIG. 6C) Box plots showing the peptide ELISA normalized OD for discovery non-endemic control serum samples (light green, n=55), discovery CM patient sera that were run with PepSeq (dark green, n=51), validation non-endemic control (light purple, n=30) and validation CM patient sera (dark purple, n=18). Welch's two sample t-test show both the CM PepSeq and CM independent have significantly increased optical density values (pvals<0.001) compared to non-endemic controls. FIG. 6D) ROC curves for peptide ELISA normalized ODs for CM samples run on PepSeq (Discovery, green), and independent validation CM samples (Validation, purple).

FIG. 7A-7C—Enriched peptides in CM and control cohorts. Box plots show the number of enriched viral positive control and Coccidioides derived peptides (Z≥10 in both replicates) in the discovery negative control and CM cohorts for human IgG (FIG. 7A), human IgM (FIG. 7B) and human IgA (FIG. 7C).

FIG. 8A-8D—Reactivity profiles for 31 IgG peptides of interest for human IgM, human IgA and PTM IgG. Box plots show the Z scores for the 31 peptides of interest identified in the human IgG screen (FIG. 8A), in human IgM (FIG. 8B), human IgA (FIG. 8C), and PTM IgG (FIG. 8D).

FIG. 9—Protein level diagnostic potential of the six proteins of interest. Receiver operating characteristic (ROC) curves for the sum of all peptides in each protein. AUCs indicated in the lower right corner.

FIG. 10—PepSeq IgG reactivity for CF antigen (CTS1). Box plots showing the Z score distributions of the max Z score for all peptides (n=45) designed from CF antigen (CTS1).

FIG. 11—Correlation of PepSeq score and CF Titer. Box plots showing the sum of the IgG Z scores for the 31 IgG peptides of interest for each clinically measured CF titer. The color of the points indicate the diagnosis of the patients if available.

FIG. 12—Correlation of PepSeq scores for the 31 IgG peptides of interest and the counts of enriched viral peptides for each sample. Scatter plot showing the sum of the IgG Z scores for the 31 IgG peptides of interest (y-axis) and the number of viral enriched peptides (x-axis). Pearson correlation coefficient is 0.353 with a p-value of 0.017. The blue line shows the linear regression and the shaded regions show the 95% confidence interval.

FIG. 13—Correlation of PepSeq scores for the 31 IgG peptides of interest and sample collection time from diagnosis. Scatter plot showing the sum of the IgG Z scores for the 31 IgG peptides of interest (y-axis) and the days between when the sample was collected and CM diagnosis (x-axis). Antibody reactivity is detected years after diagnosis CM patient samples were collected 0-3,388 days after diagnosis.

FIG. 14—Sequence conservation of reactive epitope found in IgG antigen 1. Logo plot showing the AA probability at each position of the highly reactive repetitive proline rich epitope identified in IgG antigen 1 (SEQ ID NO: 52).

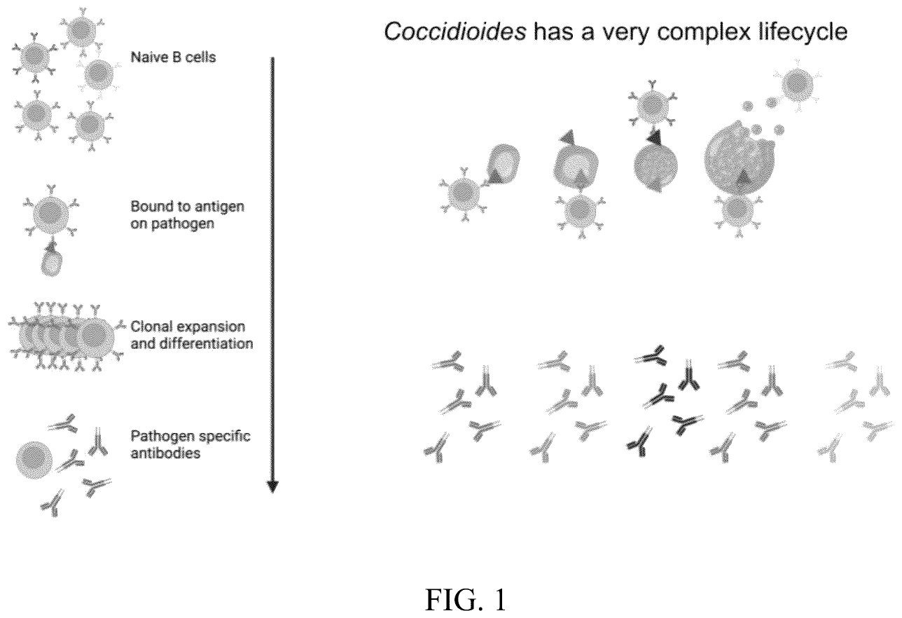

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E.A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); Marten H. Hofker and Jan van Deursen, Transgenic Mouse Methods and Protocols, 2nd edition (2011); and Yeast Genetics: Methods and Protocols (Jeffrey S. Smith & Daniel J. Burke, eds., Methods in Molecular Biology, vol. 1205, Humana Press/Springer, 2014).

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

The sample in this method is preferably a biological sample from a subject. The term “sample” or “biological sample” is used in its broadest sense. Depending upon the embodiment of the invention, for example, a sample may comprise a bodily fluid including whole blood, serum, plasma, urine, saliva, cerebral spinal fluid, semen, vaginal fluid, pulmonary fluid, tears, perspiration, mucus and the like; an extract from a cell, chromosome, organelle, or membrane isolated from a cell; a cell; genomic DNA, RNA, or cDNA, in solution or bound to a substrate; a tissue; a tissue print, or any other material isolated in whole or in part from a living subject or organism. Biological samples may also include sections of tissues such as biopsy and autopsy samples, and frozen sections taken for histologic purposes such as blood, plasma, serum, sputum, stool, tears, mucus, hair, skin, and the like. Biological samples also include explants and primary and/or transformed cell cultures derived from patient tissues.

The terms “peptide,” “polypeptide,” and “protein” are used interchangeably herein to refer to polymers of amino acids of any length, chemically or biochemically modified or derivatized amino acids, and polypeptides having modified peptide backbones. These terms also include proteins that are post-translationally modified through reactions that include glycosylation, acetylation and phosphorylation. The term “at least a portion” of a polypeptide means a portion having the minimal size characteristics of such sequences, or any larger fragment of the full length molecule, up to and including the full length molecule. For example, a portion of a polypeptide may be 4 to 15 amino acids, or may be 4 amino acids, 5 amino acids, 6 amino acids, 7 amino acids, and so on, up to a full length polypeptide. A portion of a polypeptide useful as an epitope may be as short as 4 amino acids. A portion of a polypeptide that performs the function of the full-length polypeptide would generally be longer than 4 amino acids.

The term “amino acid” refers to naturally occurring and synthetic amino acids, as well as amino acid analogs and amino acid mimetics that function in a manner similar to naturally occurring amino acids. Naturally occurring amino acids are those encoded by the genetic code, as well as those amino acids that are later modified. Unnatural amino acids are not encoded by the genetic code and can, but do not necessarily have the same basic structure as a naturally occurring amino acid. “Amino acid analogs” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to by either the three letter symbols or by the one-letter symbols recommended by the IUPAC, the IUAPC letter code are as follows: G=Glycine; A=Alanine; L-Leucine; M=Methionine; F=Phenylalanine; W=Tryptophan; K=Lysine; Q=Glutamine; E=Glutamic Acid; S=Serine; P=Proline; V=Valine; I=Isoleucine; C=Cysteine; Y=Tyrosine; H=Histidine; R=Arginine; N=Asparagine; D=Aspartic Acid; T=Threonine.

The terms “homologous” and “similar” refer to the relationship between proteins that possess a “common evolutionary origin,” including proteins from superfamilies (e.g., the immunoglobulin superfamily) and homologous proteins from different species. Such proteins (and their encoding genes) have sequence homology, as reflected by their sequence similarity, whether in terms of percent similarity or the presence of specific residues or motifs as conserved positions. In a specific embodiment, two peptide sequences are “substantially homologous or similar” when at least about 80%, or at least about 90%, or at least about 95) of the amino acids match over the defined lengths of the amino acid sequences.

The term “variant” applies to both amino acid and nucleic acid sequences. Because of the degeneracy of the genetic code, a large number of functionally identical nucleic acids encode any given protein. For instance, the codons GCA, GCC, GCG and GCU all encode the amino acid alanine. Variants may include individual substitutions, deletions or additions to a nucleic acid, peptide, polypeptide, or protein sequence which alters, adds or deletes a single amino acid or a small percentage of amino acids in the encoded sequence.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide.

Amino acids other than those indicated as conserved may differ in a protein so that the percent protein or amino acid sequence similarity between any two proteins of similar function may vary and may be, for example, from 70% to 99% as determined according to an alignment scheme. A “variant” also includes a polypeptide which has at least 60% amino acid identity as determined by BLAST or FASTA algorithms, preferably at least 75% most preferably at least 85%, and even more preferably at least 90%, and still more preferably at least 95%, and which has the same or substantially similar properties or functions as the native or parent protein to which it is compared. A particular variant is a “gain-of-function” variant, meaning a polypeptide variant in which the change of at least one given amino acid residue in a protein or enzyme improves a specific function of the polypeptide, including, but not limited to protein activity. The change in amino acid residue can be replacement of an amino acid with one having similar properties.

The term “antibody” is used herein in the broadest sense and refers generally to a molecule that contains at least one antigen binding site that immunospecifically binds to a particular antigen target of interest. The term “antibody” thus includes but is not limited to native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. The term “antibody,” thus, includes full length antibodies and/or their variants, as well as fragments thereof. Binding of an antibody to a target can cause a variety of effects, such as but not limited to where such binding modulates, decreases, increases, antagonizes, agonizes, mitigates, alleviates, blocks, inhibits, abrogates and/or interferes with at least one target activity or binding, or with receptor activity or binding, in vitro, in situ, and/or in vivo.

Antibodies (Abs) can be assigned to different classes, which differ in their biological properties, functional locations, and ability to deal with different antigens. There are five major classes of immunoglobulins (Ig): IgA, IgD, IgE, IgG, and IgM, and several of these may be further divided into subclasses (isotypes), e.g., IgG1, IgG2, IgG3, IgG4, IgA, and IgA2. The classes of immunoglobulins differ in their biological properties, functional locations, and interactions with different antigens. Immunoglobulin G (IgG), the major antibody in serum, is a heterotetrameric protein with two heavy chains and two light chains. IgG can be cleaved into three 50-kd fragments: two Fab fragments that bind antigen (F=fragment, ab=antigen binding), and an Fc fragment that crystallizes readily and does not bind antigen. The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The term “antigen” refers to any substance that triggers the immune system to produce antibodies against it. An antigen acts by stimulating the proliferation of the small number of cells that were already forming an antibody capable of recognizing the antigen.

Antibodies have specific and high affinity for the antigens that elicited their synthesis. Synthetic peptides can serve as antigens to stimulate the formation of specific antibodies. An antibody recognizes, on a large molecule, a specific group or cluster of amino acids called an epitope.

As used herein, the term “epitope” refers to a portion of an antigenic molecule to which an antibody is produced and to which the antibody will bind. For example, a “Cocci epitope” comprises the part of a Coccidioides protein to which an anti-Cocci antibody specifically binds. Epitopes can comprise linear amino acid residues (i.e., residues within the epitope are arranged sequentially one after another in a linear fashion), nonlinear amino acid residues, or both linear and nonlinear amino acid residues.

The terms “immunogen” and “immunogenic” refer to any compound or substance that is capable of eliciting an immune response in a human or non-human individual to the compound or substance, such as an antigen.

As used herein, the term “binding” refers to an attractive interaction between two molecules which results in a stable association in which the molecules are in close proximity to each other. Molecular binding can be classified into the following types: non-covalent, reversible covalent and irreversible covalent. Molecules that can participate in molecular binding include proteins, nucleic acids, carbohydrates, lipids, and small organic molecules such as pharmaceutical compounds. For example, proteins that form stable complexes with other molecules are often referred to as receptors while their binding partners are called ligands. Nucleic acids can also form stable complex with themselves or others, for example, DNA-protein complex, DNA-DNA complex, DNA-RNA complex.

As used herein, the term “specific binding” refers to the specificity of a binder, e.g., a protein or an antibody, such that it preferentially binds to a target, such as a polypeptide antigen, a receptor, or an antibody. When referring to a binding partner, e.g., protein, nucleic acid, antibody or other affinity capture agent, etc., “specific binding” can include a binding reaction of two or more binding partners with high affinity and/or complementarity to ensure selective hybridization under designated assay conditions. Typically, specific binding will be at least three times the standard deviation of the background signal. Thus, under designated conditions the binding partner binds to its particular target molecule and does not bind in a significant amount to other molecules present in the sample. Recognition by a binder or an antibody of a particular target in the presence of other potential interfering substances is one characteristic of such binding. Preferably, binders, antibodies or antibody fragments, peptides, or fusion peptides that are specific for or bind specifically to a target bind to the target with higher affinity than binding to other non-target substances. Also preferably, binders, antibodies or antibody fragments, peptides, or fusion peptides that are specific for or bind specifically to a target avoid binding to a significant percentage of non-target substances, e.g., non-target substances present in a testing sample. The binding affinity of an antibody to a target antigen, antigenic fragment, peptide, or fusion peptide, comprising the cognate epitope can be readily determined using any of a number of methods available in the art including, but not limited to, enzyme linked immunosorbent assay (ELISA). In some embodiments, binders, antibodies or antibody fragments, peptides, or fusion peptides of the present disclosure avoid binding greater than about 90% of non-target substances, although higher percentages are clearly contemplated and preferred. For example, binders, antibodies or antibody fragments, peptides, or fusion peptides of the present disclosure avoid binding about 91%, about 92%, about 93%, about 94%, about 95%, about 96%, about 97%, about 98%, about 99%, and about 99% or more of non-target substances. In other embodiments, binders, antibodies or antibody fragments, peptides, or fusion peptides of the present disclosure avoid binding greater than about 10%, 20%, 30%, 40%, 50%, 60%, or 70%, or greater than about 75%, or greater than about 80%, or greater than about 85% of non-target substances.

A target or a marker may be any molecular structure produced by a cell, expressed inside the cell, accessible on the cell surface, or secreted by the cell. A marker may be any protein, carbohydrate, fat, nucleic acid, catalytic site, or any target of these such as an enzyme, glycoprotein, cell membrane, virus, cell, organ, organelle, or any uni- or multimolecular structure or any other such structure now known or yet to be disclosed whether alone or in combination. A target may also be called a marker and the terms are used interchangeably.

A target may be represented by the sequence of amino acids, or sequence of one or more strands of a nucleic acid from which it may be derived. For example, a target may be represented by a protein sequence. Alternatively, a target may be represented by a nucleic acid sequence, the protein or peptide or the fragments thereof encoded by the nucleic acid sequence. Examples of such nucleic acids include both single stranded and double stranded nucleic acid sequences including miRNA, tRNA, siRNA, mRNA, cDNA, or genomic DNA sequences including complimentary sequences. The concept of a marker is not limited to the products of the exact nucleic acid sequence or protein sequence by which it may be represented. Rather, a marker encompasses all molecules that may be detected by a method of assessing the expression of the marker. Examples of molecules encompassed by a marker include point mutations, silent mutations, deletions, frameshift mutations, translocations, alternative splicing derivatives, differentially methylated sequences, differentially modified protein sequences, truncations, soluble forms of cell membrane associated markers, and any other variation that results in a product that may be identified as the marker. The term “target” further encompasses the products (i.e., proteins) of the gene or a gene allele thereof, whose expression or activity is directly or indirectly associated with a particular phenotype or cellular condition, or physiological characteristic.

Indirect methods of detecting a marker generally involve assessing the expression of material created from a genomic DNA template such as an RNA or protein molecule. Such expression may be assessed by any of a number of methods used currently in the art and yet to be developed. Examples include any nucleic acid detection method including the following nonlimiting examples, microarray RNA analysis, RNA in situ hybridization, RNAse protection assay, Northern blot, reverse transcription PCR, and quantitative reverse transcription PCR. Other examples include any process of detecting expression that uses an antibody including the following nonlimiting examples, flow cytometry, immunohistochemistry, ELISA, Western blot, Northwestern blot, and immunoaffinity chromatography. Antibodies may be monoclonal, polyclonal, or any antibody fragment including an Fab, F(ab)₂, Fv, scFv, phage display antibody, peptibody, multispecific ligand, or any other reagent with specific binding to a target. Other methods of assessing protein expression include the following nonlimiting examples: HPLC, mass spectrometry, protein microarray analysis, PAGE analysis, isoelectric focusing, 2-D gel electrophoresis, and enzymatic assays.

Agents that interact with a therapeutic target to result in a desirable therapeutic effect may include a pharmaceutically active ingredient or pharmaceutically acceptable salt thereof, a drug, a toxin, a chemical, a small organic molecule, a large molecule or peptide or an antibody. Large-molecule pharmaceuticals refer to pharmaceutical agents having a molecular weight greater than about 1000 Daltons, e.g., peptidic drugs, vaccines and hormones. The term “antibody” is used herein in the broadest sense and refers generally to a molecule that contains at least one antigen binding site that immunospecifically binds to a particular antigen target of interest. Antibody thus includes but is not limited to native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. The term, thus, includes full length antibodies and/or their variants as well as immunologically active fragments thereof, thus encompassing, antibody fragments capable of binding to a biological molecule (such as an antigen or receptor) or portions thereof, including, but not being limited to, Fab, Fab′, F(ab′)2, facb, pFc′, Fd, Fv or scFv (See, e.g., CURRENT PROTOCOLS IN IMMUNOLOGY, (Colligan et al., eds., John Wiley & Sons, Inc., NY, 1994-2001).

The screening or creation, identification and selection of appropriate therapeutic agent through the target identified herein can be accomplished by a variety of methods. One approach is to use structural knowledge about the target protein to design a candidate molecule with which it will precisely interact. An example would be computer assisted molecular design. A second approach is to use combinatorial or other libraries of molecules, whereby a large library of molecules is screened for inhibitory effect with regard to the target gene or protein expression, or ability to inhibit the transcriptional factor activity of the target protein. In a further example, a panel of antibodies may be screened for their ability to inhibit the target protein.

The term “monoclonal antibody” as used herein refers to an antibody obtained from a population of substantially homogeneous antibodies, i.e., the individual antibodies comprising the population are identical except for possible naturally occurring mutations that may be present in minor amounts. Various procedures known in the art may be used for the production of polyclonal or monoclonal antibodies to Cocci polypeptides or derivatives or analogs thereof. For the production of antibodies, various host animals can be immunized by injection with the antigenic polypeptide, including but not limited to rabbits, mice, rats, sheep, goats, etc. For preparation of monoclonal antibodies directed toward the Cocci polypeptides, any technique that provides for the production of antibody molecules by continuous cell lines in culture may be used. These include but are not limited to the hybridoma technique originally developed by Kohler and Milstein (Nature 256:495-497, 1975), as well as the trioma technique, the human B-cell hybridoma technique (Kozbor et al., Immunology Today 4:72, 1983; Cote et al., Proc. Natl. Acad. Sci. U.S.A. 80:2026-2030, 1983), and the EBV-hybridoma technique to produce human monoclonal antibodies (Cole et al., in Monoclonal Antibodies and Cancer Therapy, Alan R. Liss, Inc., pp. 77-96, 1985).

In the preparation of the pharmaceutical compositions comprising the antibodies described in the teachings herein, a variety of vehicles and excipients and routes of administration may be used, as will be apparent to the skilled artisan. Representative formulation technology is taught in, inter alia, Remington: The Science and Practice of Pharmacy, 19th Ed., Mack Publishing Co., Easton, Pa. (1995) and Handbook of Pharmaceutical Excipients, 3rd Ed, Kibbe, A. H. ed., Washington D.C., American Pharmaceutical Association (2000); hereby incorporated by reference in their entirety.

In other embodiments there is provided a pharmaceutical composition including an antibody or fragment as described above together with a pharmaceutically acceptable carrier, diluent or excipient. As used herein, “carrier(s)” can be used interchangeably with “excipient(s)” Carriers include any substance that may be administered with the one or more disclosed compounds with the intended purpose of facilitating, assisting, or helping the administration or other delivery of the compound. Carriers include any liquid, solid, semisolid, gel, aerosol or anything else that may be combined with the disclosed compound to aid in its administration. Examples include diluents, adjuvants, excipients, water, and oils (including petroleum, animal, vegetable or synthetic oils). Thus, the pharmaceutical compositions may generally comprise a pharmaceutically acceptable carrier and a pharmacologically effective amount of the antibodies, or mixture of antibodies. The pharmaceutical composition may be formulated as powders, granules, solutions, suspensions, aerosols, solids, pills, tablets, capsules, gels, topical creams, suppositories, transdermal patches, and other formulations known in the art.

The pharmaceutical compositions described herein may be administered by any means that enables the active agent to reach the agent's site of action in the body of the subject. The dosage administered varies depending upon factors, such as: pharmacodynamic characteristics; mode and route of administration; age, health, and weight of the recipient subject; nature and extent of symptoms; concurrent treatments; and frequency of treatment.

As used herein, the terms “administration” and “administering” of an agent to a subject include any route of introducing or delivering the agent to a subject to perform its intended function. Administration can be carried out by any suitable route, including intravenously, intramuscularly, intraperitoneally, inhalationally, intranasally, or subcutaneously. Administration includes self-administration and the administration by another.

The term “effective amount” or “therapeutically effective amount” refers to that amount of an agent or combination of agents as described herein that is sufficient to effect the intended application including, but not limited to, disease treatment and/or disease prevention. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells. The specific dose will vary depending on the particular agents chosen, the dosing regimen to be followed, whether the agent is administered in combination with other agents, timing of administration, the tissue to which it is administered, and the physical delivery system in which the compound is carried.

The terms “treatment,” “treating,” “treat,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

The terms “subject,” “individual,” and “patient” are used interchangeably herein. As used herein, the term “individual,” “patient,” or “subject” refers to any organism to which a provided composition is or may be administered, e.g., for experimental, diagnostic, prophylactic, cosmetic, and/or therapeutic purposes. For example, subject may refer to a human or a non-human animal. In some aspects, subject refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In further embodiments, the subject is a human.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Generally, some embodiments of the present invention can be used to develop diagnostics, therapies, and vaccines for Coccidioides infection based on the peptides and epitopes identified in the present disclosure. Among the various aspects of the present invention is the provision of one or more targets for diagnosing, treating, and preventing Coccidioides infection in a subject. The disclosed epitope regions of Coccidioides, including Coccidioides immitis and Coccidioides posadasii, indicate uses including serological assays, vaccines for eliciting antibodies, and predicting an individual's antibody response to a Coccidioides infection.

The invention may comprise methods for detecting the presence of exposure to, or infection by Coccidioides or a Coccidioides-associated fungal pathogen in a sample. The invention may further comprise methods of population screening, predicting immune response or disease outcome in an individual, serological assays, and neutralization assays. The invention may further comprise a method of eliciting neutralization antibodies to Coccidioides. The invention may further comprise a platform for detecting antibodies against one or more of the identified peptides.

In example embodiments, the methods, vaccines, and therapeutics may include, for example, a monoclonal antibody produced using the peptides disclosed herein.

Methods of Detecting Antibodies that Bind to a Coccidioides Protein

One aspect of the disclosure encompasses methods of detecting the presence of an antibody that binds to a Coccidioides protein in a sample from a subject. In example embodiments, the subject is tested for prior exposure to Coccidioides or a current Coccidioides infection. In example embodiments, the subject is monitored for a current Coccidioides infection. In example embodiments, the antibodies are generated in a subject exposed to Coccidioides.

In example embodiments, an antibody is detected that binds to a peptide selected from SEQ ID NO: 1-50, or variants thereof having at least 85% sequence identity to any of SEQ ID NO: 1-50. In example embodiments, a combination of one or more peptides as described herein are used to detect antibodies that bind to a Coccidioides protein.

In example embodiments, the combination of peptides is used in an assay designed to detect binding of the peptides to an antibody present in a patient sample. In example embodiments, a combination of one or more peptides as described herein is used to detect antibodies that bind to a Coccidioides protein. In example embodiments, the combination comprises two or more peptides selected from SEQ ID NO: 1-50, or variants thereof having at least 85% sequence identity to any of SEQ ID NO: 1-50. In various embodiments, the combination comprises peptides derived from multiple distinct Coccidioides proteins and/or multiple epitopes within a single Coccidioides protein. In such embodiments, the peptides may be selected without limitation as to the number of peptides selected from any individual protein or the number of distinct proteins represented in the combination.

In example embodiments, the combination comprises peptides selected to represent epitopes identified through proteome-wide serological analysis, including peptides that demonstrate diagnostic enrichment relative to non-endemic controls. In preferred embodiments, the combination comprises a defined panel of peptides selected from SEQ ID NO: 1-50 that together provide improved sensitivity, specificity, or robustness of antibody detection compared to detection using a single peptide or antigen alone. In example embodiments, the peptides comprise SEQ ID NO: 1, 3, 7, 13, 15, 17, 24, 26, 29, 32, 36, 38, 39, 40, 43, and 44, or variants thereof having at least 85% sequence identity.

In example embodiments, the combination further comprises one or more additional epitopes derived from Coccidioides proteins identified in prior studies or from previously known Coccidioides antigens, thereby enabling multiplexed detection of antibodies directed to both newly identified and previously characterized epitopes.

In exemplary embodiments, the combination comprises a panel of 2-20 peptides selected from SEQ ID NOS: 1-50 or variants thereof having at least 85% sequence identity, which panel is suitable for use in a multi-peptide ELISA for serological detection of Coccidioides infection. In various embodiments, the peptides are provided as discrete synthetic peptides and are not present as part of a full-length Coccidioides protein. In example embodiments, the peptides comprise SEQ ID NO: 1, 3, 7, 13, 15, 17, 24, 26, 29, 32, 36, 38, 39, 40, 43, and 44, or variants thereof having at least 85% sequence identity.

In example embodiments, the combination comprises one or more peptides derived from SOWgp and one or more peptides derived from at least one additional Coccidioides protein. In example embodiments, the additional protein is selected from IgG antigen 1, IgG antigen 2, IgG antigen 3, IgG antigen 4, or IgG antigen 5. In example embodiments, the combination comprises peptides selected from SOWgp and IgG antigens 1-3. In further embodiments, the combination comprises peptides selected from IgG antigens 1-5, optionally in the absence of SOWgp-derived peptides.

### ELISA

In example embodiments, detection of antibodies that bind to a Coccidioides protein is performed using an enzyme-linked immunosorbent assay (ELISA). In such embodiments, one or more peptides selected from SEQ ID NOS: 1-50, or variants thereof having at least 85% sequence identity to any of SEQ ID NOS: 1-50, are used as capture antigens to detect antibodies present in a biological sample obtained from a subject.

In various embodiments, the ELISA comprises contacting a biological sample with one or more peptides under conditions sufficient to permit binding of antibodies in the sample to the peptides, followed by detection of peptide-antibody complexes. The biological sample may comprise serum, plasma, or another suitable bodily fluid.

In example embodiments, the peptides are immobilized on a solid support suitable for ELISA, including but not limited to microtiter plates, wells, or other substrates. In example embodiments, the peptides are immobilized directly on the solid support, while in other embodiments the peptides are indirectly immobilized through interaction with a capture reagent.

In example embodiments, one or more peptides are labeled with biotin and immobilized on streptavidin- or avidin-coated plates. In further embodiments, the peptides are provided with other labels or functional groups that facilitate immobilization or detection, including affinity tags, linkers, or haptens. In various embodiments, the peptides are provided as discrete synthetic peptides and are not present as part of a full-length Coccidioides protein.

In example embodiments, bound antibodies are detected using a labeled secondary antibody that specifically binds to a constant region of the subject antibody. In example embodiments, the secondary antibody is specific for IgG, IgM, IgA, or combinations thereof.

In example embodiments, the secondary antibody comprises an enzymatic label selected from horseradish peroxidase (HRP), alkaline phosphatase, or other suitable enzymes. Following addition of an appropriate substrate, a detectable signal is generated, including a colorimetric, fluorescent, or chemiluminescent signal, which is measured to determine the presence or amount of antibodies that bind to the peptides.

In example embodiments, detection of antibody binding above a predetermined threshold indicates exposure to or infection with Coccidioides.

In example embodiments, the ELISA is performed in a direct ELISA format, while in other embodiments the ELISA is performed in an indirect or sandwich format. In further embodiments, the ELISA is conducted in a high-throughput or automated format.

### Flow Cytometry-Based Detection

In example embodiments, detection of antigen-antibody complexes may be performed using flow cytometry. In such embodiments, peptides disclosed herein may be immobilized on solid supports compatible with flow cytometric analysis, including microparticles or beads.

In example embodiments, peptides comprising an amino acid sequence selected from SEQ ID NOS: 1-50, or variants thereof having at least 85% sequence identity, are coupled to beads, such as polymeric, magnetic, latex, agarose, or polystyrene beads. Beads may have reactive surface chemistries that permit covalent or non-covalent attachment of peptides, including but not limited to amine-reactive, thiol-reactive, carboxyl-reactive, streptavidin-coated, or affinity-based coupling chemistries.

In example embodiments, peptides are synthesized with a functional group, such as biotin, a thiol group, an amine, or a linker moiety, to facilitate attachment to the bead surface. For example, biotinylated peptides may be immobilized on streptavidin-coated beads, or peptides may be covalently coupled to carboxylated beads using carbodiimide chemistry.

Following immobilization of peptides on beads, the peptide-coupled beads are contacted with a biological sample obtained from a subject, such as whole blood, serum, or plasma, under conditions sufficient to permit binding of antibodies present in the sample to the immobilized peptides. Unbound components may be removed by washing.

In example embodiments, bound antibodies are detected using labeled secondary antibodies that bind to the Fc region of the subject antibody. Such labels may include fluorescent dyes suitable for detection by flow cytometry. Detection may be isotype-specific and may include, for example, anti-human IgG, anti-human IgM, or anti-human IgA secondary antibodies.

The bead-antibody complexes are analyzed using a flow cytometer, wherein individual beads are interrogated to detect fluorescence associated with antibody binding. The intensity of detected fluorescence is indicative of the presence or amount of antibody bound to a peptide.

In example embodiments, different bead populations may be distinguished based on size, internal dye intensity, or other bead-specific characteristics, permitting multiplexed detection of antibodies binding to multiple different peptides simultaneously within a single assay. In such multiplexed embodiments, each bead population may be coupled to a different peptide or epitope disclosed herein.

Detection may be qualitative, semi-quantitative, or quantitative, and results may be expressed as fluorescence intensity, percentage of positive beads, or other flow cytometric metrics.

Flow cytometry-based peptide bead assays provide a rapid, sensitive, and scalable method for detecting antibodies to Coccidioides antigens. In example embodiments, such assays permit simultaneous detection of antibodies to multiple epitopes, increased analytical specificity, or reduced sample volume requirements.

### Immunoaffinity Chromatography Using Peptide-Immobilized Supports

In example embodiments, detection of antibodies specific to Coccidioides may be performed using immunoaffinity chromatography. In such embodiments, one or more peptides comprising an amino acid sequence selected from SEQ ID NOS: 1-50, or variants thereof having at least 85% sequence identity, are immobilized on a solid support.

In example embodiments, the solid support comprises a chromatography matrix, column, resin, bead, or membrane suitable for affinity-based separations. The peptides may be immobilized by covalent attachment or affinity interactions, including but not limited to amine coupling, thiol coupling, carbodiimide chemistry, or biotin-streptavidin interactions.

A biological sample obtained from a subject, such as serum or plasma, is passed over or through the peptide-immobilized support under conditions sufficient to permit binding of antibodies present in the sample to the immobilized peptides. Unbound material may be removed by washing, and the presence of bound antibodies may be detected directly or following elution from the support.

In example embodiments, bound antibodies are detected using labeled secondary antibodies, enzymatic detection, spectroscopic methods, or other immunological detection techniques. In other embodiments, antibodies bound to the peptide-immobilized support are eluted and subsequently analyzed or quantified.

Accordingly, immunoaffinity chromatography provides a method for selectively capturing and detecting antibodies specific to Coccidioides epitopes disclosed herein.

### Methods of Treating a Coccidioides Infection

One aspect of the disclosure encompasses methods of treating a subject that is determined to have a Coccidioides infection according to any of the detection methods described herein. In example embodiments, the methods disclosed herein further comprise treating the subject following detection of antibodies to Coccidioides peptides as described herein. Detection of antigen-antibody complexes may inform clinical decision-making regarding initiation, adjustment, or cessation of antifungal therapy. As used herein, “treatment” includes therapeutic, prophylactic, suppressive, or disease modifying interventions intended to reduce fungal load, alleviate symptoms, prevent disease progression, or reduce the risk of dissemination.

In example embodiments, detecting antibodies that bind to a Coccidioides protein is used as a diagnostic assay that can be used to guide treatment of a subject. In example embodiments, the diagnostic peptides and immunoassays disclosed herein may be used to monitor treatment response or disease progression. In example embodiments, the assays can distinguish between non-endemic samples and CM positive samples. In example embodiments, the methods and compositions disclosed herein may be used in connection with the treatment of a Coccidioides infection in a subject. In some embodiments, treatment is initiated following identification or suspicion of Coccidioides infection, including infection diagnosed or monitored using the diagnostic methods described herein. The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). The term “monitoring” generally refers to the follow-up of a disease or a condition in a subject for any changes which may occur over time.

In example embodiments, a Coccidioides infection is treated in a subject according to any known treatment. In example embodiments, treatment of a Coccidioides infection comprises administration of one or more antifungal agents. In example embodiments, the antifungal agent is an azole antifungal, including but not limited to fluconazole, itraconazole, voriconazole, posaconazole, or isavuconazole. In example embodiments, azole antifungals are administered orally and are suitable for the treatment of pulmonary, chronic, or non-severe extrapulmonary Coccidioides infection.

In example embodiments, treatment comprises administration of amphotericin B or a pharmaceutically acceptable formulation thereof. Amphotericin B is a powerful, broad-spectrum polyene antifungal medication used to treat severe, life-threatening systemic fungal infections and certain protozoan infections. It acts by binding to ergosterol in fungal cell membranes, causing cell leakage and death. Due to high toxicity (nephrotoxicity) and infusion-related reactions, it is often reserved for severe cases, with liposomal formulations used to reduce side effects. In example embodiments, amphotericin B is administered intravenously, in cases of severe infection, in disseminated disease, in immunocompromised subjects, or prior to or in combination with azole therapy. In example embodiments, amphotericin B therapy is followed by maintenance or suppressive therapy with an azole antifungal.

In example embodiments, treatment comprises combination therapy or sequential therapy, wherein a subject receives a first antifungal agent followed by a second antifungal agent, or concurrent administration of two or more antifungal agents. Sequential or combination therapy may be selected based on disease severity, response to treatment, subject risk factors, or site of infection.

In example embodiments, antifungal therapy is administered for a period of weeks, months, or longer durations as clinically indicated. In example embodiments, treatment duration is adjusted based on clinical response, serological monitoring, imaging results, or other disease markers. In example embodiments, treatment further comprises supportive care, which may include symptom management, hospitalization, or additional therapeutic interventions as appropriate.

### Immunogenic Compositions

One aspect of the disclosure encompasses methods of eliciting an immune response in a subject by administering to the subject an immunogenic composition. The methods may include recruiting an antifungal antibody in the subject, to protect the subject against a Coccidioides infection. The methods, vaccines, and therapeutics may include including a peptide comprising an amino acid sequence selected from the group consisting of SEQ ID NOS: 1-50, or variants thereof having at least 85% sequence identity to any of SEQ ID NOS: 1-50. In example embodiments, the peptides contribute to eliciting an immune response specific to Coccidioides infection as part of a multivalent or combination vaccine. In example embodiments, the composition comprises two or more peptides, including combinations of peptides derived from multiple distinct Coccidioides proteins and/or multiple epitopes within a single Coccidioides protein. In various embodiments, the peptides may be selected without limitation as to the number of peptides selected from any individual protein or the number of distinct proteins represented in the immunogenic composition. In example embodiments, one or more peptides included in an immunogenic composition comprise an amino acid sequence selected from SEQ ID NO: 1, 3, 7, 13, 15, 17, 24, 26, 29, 32, 36, 38, 39, 40, 43, and 44, or variants thereof having at least 85% sequence identity. In example embodiments, one or more peptides included in an immunogenic composition comprise an amino acid sequence selected from SEQ ID NO: 2-4, 8-9, 11-18, 23-25, 27, 29, 30-33, 35, 37-41, 43-44, and 48, or variants thereof having at least 85% sequence identity.

In example embodiments, the immunogenic composition comprises between one and forty-eight peptides selected from SEQ ID NOS: 1-50, or variants thereof having at least 85% sequence identity. In example embodiments, the composition comprises two or more peptides, at least five peptides, at least ten peptides, or at least twenty peptides selected from SEQ ID NOS: 1-50.

In various embodiments, the peptides are provided as discrete peptide sequences and are not present as part of a full-length native Coccidioides protein.

In example embodiments, two or more peptides selected from SEQ ID NOS: 1-50, or variants thereof having at least 85% sequence identity, are linked together in a single polypeptide. In example embodiments, the peptides are expressed as part of a fusion protein, optionally separated by linker sequences. In example embodiments, the fusion protein further comprises one or more carrier sequences, scaffold proteins, secretion signals, or expression-enhancing domains. In various embodiments, the fusion protein is produced recombinantly.

In example embodiments, the immunogenic composition further comprises one or more additional antigens derived from Coccidioides proteins identified in prior studies or previously known Coccidioides antigens. In further embodiments, the immunogenic composition comprises one or more antigens derived from non-Coccidioides fungal pathogens.

In example embodiments, the immunogenic composition further comprises a pharmaceutically acceptable carrier, excipient, or adjuvant. Representative adjuvants include, without limitation, alum-based adjuvants, oil-in-water emulsions, liposomes, toll-like receptor agonists, or combinations thereof.

In example embodiments, the immunogenic composition is formulated for administration by injection, mucosal delivery, intranasal delivery, or other suitable routes of administration.

One of skill in the art from this disclosure and the knowledge in the art will appreciate that there are a variety of ways in which to produce such Coccidioides specific antigens. In general, such Coccidioides specific antigens may be produced either in vitro or in vivo. Coccidioides specific antigens may be produced in vitro as peptides or polypeptides, which may then be formulated into a vaccine or immunogenic composition and administered to a subject. As described in further detail herein, such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, Coccidioides specific antigens may be produced in vivo by introducing molecules (e.g., DNA, RNA, viral expression systems, and the like) that encode Coccidioides specific antigens into a subject, whereupon the encoded Coccidioides specific antigens are expressed. The methods of in vitro and in vivo production of antigens are also further described herein as it relates to pharmaceutical compositions and methods of delivery.

In example embodiments, an immunogenic composition comprises 1, 5, 10, 15, 20, 25, 30, 35, 40, 45, 50, 55, 60, 65, 70, 75, 80, 85, 90, 95, or more than 100 peptides. Targeting as many epitopes as practically possible takes advantage of the enormous capacity of the immune system, prevents the opportunity for immunological escape by down-modulation of a particular immune targeted gene product, and compensates for the known inaccuracy of epitope prediction approaches. Synthetic peptides provide a particularly useful means to prepare multiple immunogens efficiently and to rapidly translate epitopes to an effective vaccine or immunogenic composition. Peptides can be readily synthesized chemically and easily purified utilizing reagents free of contaminating bacteria or animal substances. The small size allows a clear focus on the immunogenic region of the protein and also reduces irrelevant antigenic competition from other components.

Peptides may be made by any technique known to those of skill in the art, including the expression of proteins, polypeptides or peptides through standard molecular biological techniques, the isolation of proteins or peptides from natural sources, in vitro translation, or the chemical synthesis of proteins or peptides. Peptides can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963). Alternatively, a nucleic acid (e.g., a polynucleotide) encoding a peptide of the invention may be used to produce the peptide in vitro. Many exemplary systems exist that one skilled in the art could utilize (e.g., Retic Lysate IVT Kit, Life Technologies, Waitham, MA). An expression vector capable of expressing a polypeptide can also be prepared. Expression vectors for different cell types are well known in the art and can be selected without undue experimentation.

In example embodiments, the antigenic peptides of the invention may be encoded and expressed in vivo using a viral based system (e.g., an adenovirus system, an adeno associated virus (AAV) vector, a poxvirus, or a lentivirus).

The present invention also contemplates the use of nucleic acid molecules as vehicles for delivering immunogens to a subject, in vivo, in the form of, e.g., DNA/RNA vaccines.

mRNA Vaccines

In example embodiments, the present disclosure provides an mRNA vaccine comprising one or more mRNA molecules encoding one or more peptide sequences selected from SEQ ID NOS: 1-50, or variants thereof having at least 85% sequence identity to any of SEQ ID NOS: 1-50. Messenger RNA (mRNA) vaccine technology provides a versatile platform for in vivo expression of antigenic peptides and proteins, enabling induction of both humoral and cellular immune responses without the need for administration of full-length recombinant proteins. mRNA vaccines have been validated clinically through widespread use of nucleoside-modified mRNA vaccines against SARS-COV-2, which demonstrated that mRNA encoding pathogen-derived antigens, when delivered in lipid nanoparticle (LNP) formulations, can safely and effectively elicit protective immune responses in humans (see, e.g., Pardi N, Krammer F. mRNA vaccines for infectious diseases-advances, challenges and opportunities. Nat Rev Drug Discov. 2024; 23 (11): 838-861). mRNA vaccines are typically formulated as in vitro-transcribed mRNA encoding an antigen of interest, optionally incorporating nucleoside modifications to reduce innate immune sensing and enhance translational efficiency, and packaged in lipid nanoparticles to facilitate cellular uptake and cytosolic delivery (see, e.g., Gote V, Bolla P K, Kommineni N, et al. A Comprehensive Review of mRNA Vaccines. Int J Mol Sci. 2023; 24 (3): 2700).

### Kits

One aspect of the disclosure encompasses kits for detection of an antibody that binds to a Coccidioides protein. The term “kit” as used throughout this specification refer to a product containing components necessary for carrying out the specified methods (e.g., methods for detecting as taught herein), packed so as to allow their transport and storage. Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports. Typically, the kits may also include instructions for use thereof, such as on a printed insert or on a computer readable medium.

In example embodiments, a combination of peptides is included in a kit designed to detect binding of the peptides to an antibody present in a patient sample. In example embodiments, a combination of one or more peptides as described herein is included in a kit to detect antibodies that bind to a Coccidioides protein. In example embodiments, the combination comprises two or more peptides selected from SEQ ID NO: 1-50, or variants thereof having at least 85% sequence identity to any of SEQ ID NO: 1-50. In various embodiments, the combination comprises peptides derived from multiple distinct Coccidioides proteins and/or multiple epitopes within a single Coccidioides protein. In such embodiments, the peptides may be selected without limitation as to the number of peptides selected from any individual protein or the number of distinct proteins represented in the combination.

In example embodiments, the combination comprises peptides selected to represent epitopes identified through proteome-wide serological analysis, including peptides that demonstrate diagnostic enrichment relative to non-endemic controls. In preferred embodiments, the combination comprises a defined panel of peptides selected from SEQ ID NO: 1-50 that together provide improved sensitivity, specificity, or robustness of antibody detection compared to detection using a single peptide or antigen alone. In example embodiments, the peptides comprise SEQ ID NO: 1, 3, 7, 13, 15, 17, 24, 26, 29, 32, 36, 38, 39, 40, 43, and 44, or variants thereof having at least 85% sequence identity.

In example embodiments, the combination further comprises one or more additional epitopes derived from Coccidioides proteins identified in prior studies or from previously known Coccidioides antigens, thereby enabling multiplexed detection of antibodies directed to both newly identified and previously characterized epitopes.

In example embodiments, the combination comprises a panel of 2-20 peptides selected from SEQ ID NOS: 1-50 or variants thereof having at least 85% sequence identity, which panel is suitable for use in a multi-peptide ELISA for serological detection of Coccidioides infection. In various embodiments, the peptides are provided as discrete synthetic peptides and are not present as part of a full-length Coccidioides protein. In example embodiments, the peptides comprise SEQ ID NO: 1, 3, 7, 13, 15, 17, 24, 26, 29, 32, 36, 38, 39, 40, 43, and 44, or variants thereof having at least 85% sequence identity.

In example embodiments, the combination comprises one or more peptides derived from SOWgp and one or more peptides derived from at least one additional Coccidioides protein. In example embodiments, the additional protein is selected from IgG antigen 1, IgG antigen 2, IgG antigen 3, IgG antigen 4, or IgG antigen 5. In example embodiments, the combination comprises peptides selected from SOWgp and IgG antigens 1-3. In further embodiments, the combination comprises peptides selected from IgG antigens 1-5, optionally in the absence of SOWgp-derived peptides.

In example embodiments, the peptides included in the kit are immobilized on a solid support or are otherwise configured for use in a serological assay. In example embodiments, the kit further comprises one or more reagents selected from buffers, wash solutions, detection reagents, labeled secondary antibodies, enzyme substrates, solid supports, instructions for use, or combinations thereof. In example embodiments, the solid support comprises a plate, bead, membrane, slide, or other substrate suitable for immobilizing peptides for antibody detection.

In example embodiments, one or more peptides in the kit are modified to include a detectable or immobilization-facilitating label. In example embodiments, the label comprises biotin. In such embodiments, the biotin-labeled peptides may be configured for capture on streptavidin- or avidin-coated solid supports.

In further embodiments, the peptides are labeled with a detectable label selected from fluorescent labels, chemiluminescent labels, enzymatic labels, colorimetric labels, affinity tags, haptens, or combinations thereof. Representative labels include, without limitation, fluorophores, horseradish peroxidase, alkaline phosphatase, nanoparticles, beads, or epitope tags.

In some embodiments, the label is attached to the peptide at the N-terminus, the C-terminus, or via an internal residue or linker. In example embodiments, the peptides are synthesized with a functional group or linker to facilitate attachment of the label. In various embodiments, labeled peptides are provided individually or as part of a peptide mixture or panel.

In some embodiments, the labeling strategy is selected to permit use of the kit in plate-based, bead-based, lateral-flow, or multiplexed immunoassay formats.

In further embodiments, the peptides are unlabeled and are configured for indirect detection through binding to a labeled secondary reagent.

Further embodiments are illustrated in the following Examples which are given for illustrative purposes only and are not intended to limit the scope of the invention.

### EXAMPLES

### Example 1—Proteome-Wide Discovery of Novel Diagnostic Antigens for Coccidioidomycosis

In this example, we conduct the first Coccidioides proteome-wide serological analysis across multiple isotypes to identify linear epitopes recognized antibodies during CM infection at an unprecedented scale. We created a novel 244,000-member peptide library, Coccidioides whole proteome (CWP), covering >96% of all 11-mers in the publicly available C. immitis and C. posadasii proteomes. We used the novel 244,000-member peptide library (“PepSeq”) to assess reactivity profiles for IgG, IgA and IgM antibodies in serum samples from human subjects with confirmed Coccidioides infections (CM) or healthy controls from non-endemic areas (Louisiana), as well as IgG profiles for plasma samples from naturally infected pigtail macaques (PTM) (infected in the endemic area).

The multiplexed peptide assay (“PepSeq”) platform is a fully in vitro and highly multiplexed technology that generates and uses custom libraries of DNA barcoded peptides to quantitatively probe serum for pathogen specific antibodies²⁰. We have previously used this technology to characterize antibody responses to large numbers of viruses and bacteria at epitope-level resolution through the lenses of natural infection and vaccination. We applied this technology to test the hypothesis that the Coccidioides proteome contains undiscovered antigenic epitopes that may be useful in future CM diagnostic approaches. Using this platform, we have designed the first proteome-wide peptide library, which covers both species of Coccidioides. Presented here is the first Coccidioides proteome-wide serological analysis across multiple antibody isotypes (IgG, IgA, IgM) to identify linear epitopes recognized by antibodies during CM infection at an unprecedented scale. Our findings revealed human antibody reactivity to previously known antigenic proteins (e.g., spherical outer wall glycoprotein (SOWgp)) and identify five novel proteins that have not been previously described.

We next created a novel multi-peptide ELISA based on Coccidioides spp. specific significantly reactive peptides identified in our IgG screen. Multi-peptide ELISAs were performed on the serum samples described and an independent sample set to test the diagnostic utility of the discovered antigens. We were able to robustly discriminate between human CM and control patient sera in a validation cohort, demonstrating that the epitopes discovered using this approach can be used for diagnostic purposes beyond the discovery cohort and may be useful for the improvement of CM diagnostic approaches. The targets identified can be used into a serology based diagnostic kit. These targets could be synthesized as peptides rather than whole proteins. Peptides are advantageous as they are simpler molecules making them more affordable to make and less error prone. No other current Coccidioidomycosis diagnostic uses this approach.

### Methods

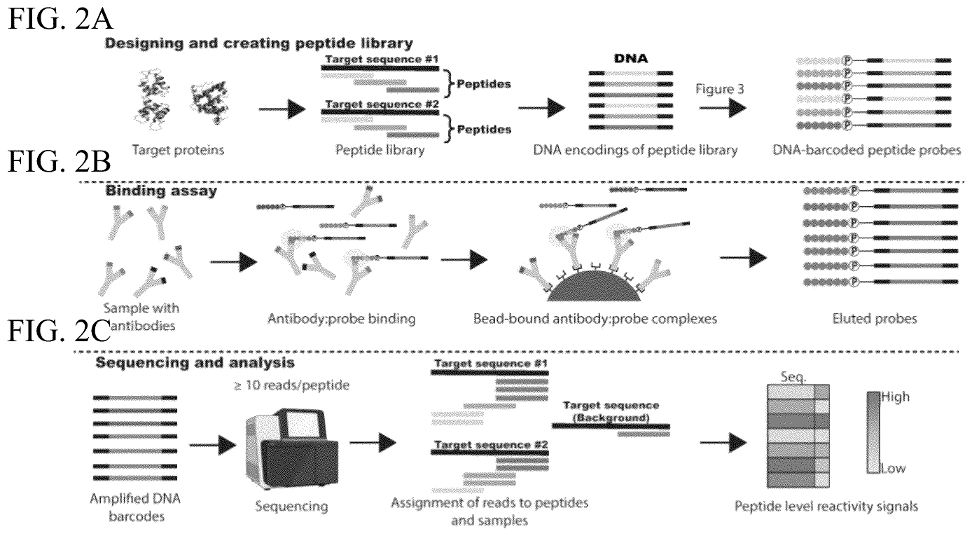

A novel 244,000-member 30mer peptide library, Coccidioides whole proteome (CWP), covering >96% of all 11-mers in the publicly available C. immitis and C. posadasii proteomes was created (see, FIG. 2). Serum from 71 CM patient sera and 60 control sera from a non-endemic region in the US were assayed against the library; then, IgG, IgM and IgA antibody reactivity was mapped at the protein and epitope levels. Due to variable levels of background for each individual peptide, we used a dynamic thresholding approach where a threshold was calculated for each peptide. The threshold was set three standard deviations above the mean of the 97% highest density interval of the non-endemic control samples. A pilot multi-peptide ELISA was designed based on IgG epitopes which were significantly reactive in Coccidioidomycosis patients sample set compared to the controls. An independent sample set of CM patients was included in ELISA runs. A welches two sample t-test was used to compare OD values.

### Serum/Plasma Samples

Serum samples collected from healthy patients outside the endemic range for CM (Louisiana, USA) were used as the presumed negative controls and were divided into the discovery negative control cohort (n=60) and validation negative control cohort (n=30). Serum and plasma samples from patients with coccidioidomycosis were divided into two cohorts: discovery (n=70, analyzed with PepSeq) and validation (n=18, analyzed with ELISA). The discovery CM cohort was recruited under IRB number 1712117680A003 by the University of Arizona biobank. The IRB number STUDY00002075 allowed us to utilize biobanked samples from the University of Arizona. The validation CM cohort was obtained from the UC Davis Coccidioidomycosis Serology Laboratory under IRB number 1661992 to validate ELISA performance. Plasma samples were obtained from naturally infected pigtail macaques (n=33) at the Washington National Primate Laboratory (WaNPRC). Animals at the WaNPRC Arizona Breeding Colony are housed in Mesa, AZ (IACUC (4551-05). As a part of routine animal care, animals undergo thorough semi-annual exams which include the collection of sera to test for Coccidioides spp. seropositivity. Excess sera from these exams are stored in a repository at WaNPRC, and were shared for these experiments. Plasma samples from WaNPRC PTM animals born and raised in Seattle, WA (n=17) were used as the controls in these experiments (IACUC 4551-04).

### Design of Coccidioides Whole Proteome Peptide Library

For the CWP PepSeq library, we targeted all annotated proteins for each Coccidioides species: C. immitis and C. posadasii. Specifically, we included all of the proteins annotated in the two publicly available genomes with the highest sequencing depth for each Coccidioides species, as well as a few additional sequences for known Coccidioides spp. antigens. For C. posadasii, we included Silveira 2022 (GCA_018416015.2) and RMSCC 3488 (GCA_000150055.1); and for C. immitis, we included WA_211 (GCA_004115165.2) and RMSCC 2394 (GCA_000149895.1). Annotated protein sequences from these four Coccidioides spp. genomes were downloaded from GenBank on Jul. 20, 2023 (Table 1). We confirmed the inclusion of all known Coccidioides spp. antigen protein sequences from previously reported studies²⁷, IEDB and the currently used diagnostic proteins.

TABLE 1
Coccidioides genomes used in the design of the CWP library

| | | | | Release | Protein- | Genome |
|---|---|---|---|---|---|---|
| Genbank Assembly | Species | Strain | Level | Date | coding Genes | coverage |
| GCA_018416015.2 | C. posadasii | Silveira | Complete | February, 2022 | 8,299 | 475x |
| GCA_000150055.1 | C. posadasii | RMSCC 3488 | Scaffold | July, 2007 | 9,897 | 8x |
| GCA_004115165.2 | C. immitis | WA_211 | Scaffold | July, 2019 | 7,815 | 62x |
| GCA_000149895.1 | C. immitis | RMSCC 2394 | Scaffold | November, 2006 | 10,403 | 8x |

Peptides were designed from these target proteins using a combined set cover and sliding window approach²⁰. First, we generated clusters of similar protein sequences from the four focal genomes using USEARCH (70% identity threshold) 28. We then selected representative peptides for each cluster, each of which was 30 amino acids (AA) long (github.com/LadnerLab/Library-Design/tree/master/SW_SC). Collectively, these peptides covered ≥95% of the unique 9mers (proxy for linear epitopes) contained in the proteomes of these four genomes and manually curated known antigens described above. Next, we removed redundant peptides using a custom script (github.com/LadnerLab/Library-Design/blob/master/extensions/kmerRedundancy.py) that steps through kmer sizes from 30 AA to 8 AA and removes any peptides that do not contain any unique kmers. This resulted in a 30 AA peptide library containing 243,135 peptides designed from C. immitis and C. posadasii proteins. Our final design covered 96% of the unique AA 9mers in all 11 of the publicly available Coccidioides spp. genomes. Finally, we added 865 positive control viral peptides (designed from 40 different virus species), which previous studies have shown to be commonly recognized by IgG antibodies in human samples^{21,24}.

### PepSeq Library Synthesis and Antibody Binding Assays

The CWP library was encoded as 244,000 128 base pair DNA oligonucleotides using custom scripts²⁰, and were sourced from Agilent (SurePrint oligo pool). These oligonucleotides were used as the starting material to synthesize the CWP PepSeq library of DNA-barcoded peptides following methods previously described²⁰. Briefly, the oligonucleotide library was PCR-amplified and then used to generate mRNA in an in vitro transcription reaction. The mRNA products were ligated to hairpin oligonucleotide adapters bearing a puromycin molecule tethered by a PEG spacer and used as templates in an in vitro translation reaction, resulting in peptides covalently bound to their associated mRNA. Finally, a reverse transcription reaction, primed by the adaptor hairpin, was used to generate a cDNA tag, and the original mRNA was removed using RNAse. To perform serological assays, the resulting CWP DNA-barcoded peptide library was added to diluted plasma or serum and incubated overnight. The binding reaction was then applied to pre-washed isotype specific (IgG, IgM or IgA) capture beads. IgG was captured with streptococcal protein G-bearing beads (ThermoFisher Cat #10004D). IgA and IgM were captured with polyclonal antibodies specific for these human isotypes. IgA and IgM specific beads were generated by incubating biotinylated anti-IgM (Biolegend #314504) or anti-IgA (Invitrogen, #MA5-27919) antibodies in excess with streptavidin beads (Thermo Fisher, #6502). Following capture, bead bound antibodies were repeatedly washed, bound PepSeq probes were eluted, and the associated DNA barcodes were amplified and indexed for sequencing. Following PCR cleanup, products were pooled, quantified, and sequenced using an Illumina NextSeq or NovaSeq instrument. All samples were assayed in duplicate.

### PepSeq Data Analysis

To identify peptides with diagnostic potential for CM, we compared antibody reactivity profiles for 70 confirmed CM patients and 60 presumed negative blood bank controls. For this comparison, we focused on the subset of peptides (n=884) that showed reactivity in at least five samples, regardless of their cohort of origin (Z score≥10 in ≥5 samples). For each, a peptide-specific Z score threshold was calculated as three standard deviations above the mean Z score of the discovery negative control samples. These peptide-specific thresholds were used to count the number of positive and negative samples in each cohort. To identify peptides with significantly more positives among our CM cohort, we used two-sided Fisher's Exact tests with a Benjamini-Hochberg multiple test correction. Peptides of interest were identified as those with a corrected p-value≤0.05. To assess our false discovery rate, we followed an identical procedure to also identify peptides with higher reactivity in 1) our control cohort compared to our CM cohort and 2) datasets with randomly permuted cohort assignments (n=1000). For each protein that contained at least one significant peptide, we predicted the protein's cellular localization using DeepLoc2.1²⁹ with default parameters. For each protein, we input the sequence accession identifier that was used during the design process. The FungiDB database was used to search for gene/protein names, predicted function and gene expression data³⁰. Sequence accession numbers were also input to NCBI BLASTP with clusterNR as the database to look for regions of similarity³¹.

To assess the diagnostic performance, within our discovery cohort, of the peptides of interest detected in our IgG screen, we generated receiver operating characteristic (ROC) curves, both for individual peptides and the Z score sum across multiple peptides, with Z score thresholds ranging from −2.3 to 3,572. To determine the smallest subset of peptides that yielded the largest AUC, we utilized forward and backward selection models, requiring a minimum increase in AUC of 0.001 when adding (or subtracting) a peptide. At each step, the peptide that resulted in the largest AUC increase was selected for inclusion (or exclusion).

To identify significant differences in the summed Z score of our 31 IgG peptides of interest and CF titer while controlling for gender, diagnosis, sample location, year of diagnosis and viral peptide counts, we utilized a GLM implemented in Python using statsmodels v0.14.1³². Specifically, we fit a Gaussian GLM with a single dependent variable (summed Z score) and six independent variables (CF titer [continuous], gender [categorical], diagnosis [categorical], sample location [categorical], shipment [categorical], and enriched viral peptide counts [continuous]).

### Multi-Peptide ELISA's

From an initial PepSeq screen of n=101 human samples, a set of 30 peptides of interest were identified and clustered into 11 groups based on sequence homology using Clustal Omega³³. For clusters containing more than one sequence, the sequence with the highest Z score across all CM samples in each cluster was selected, if the cluster contained multiple potential epitopes, the peptide with the highest Z score in CM samples for each epitope was selected, resulting in 16 peptides being selected for inclusion in a novel, multi-epitope ELISA. C-terminus biotinylated, 30 amino acid peptides were obtained from Thermo Fisher (see, Table 6), and these were used to create a peptide pool at equimolar concentrations.

With this peptide pool as the antigen, a direct ELISA protocol was performed using the following materials and steps. NeutrAvidin coated plates (Thermo) were incubated at 4° C. overnight with 1 μg/mL of the peptide pool. Wells were blocked using a 1% Blocker BSA solution (Thermo) prior to sample introduction. Diluted (1:500) patient sample was added to each peptide containing well (Sample OD). Additionally, for each donor, diluted (1:500) patient sample was added to control wells without peptide (Serum OD). All samples and controls were run in duplicate and for each plate two wells with 1 μg/mL of the peptide pool but without serum were included (Peptide OD). Goat anti-Human IgG-HRP conjugated antibody (Abcam) was used as the detection antibody, followed by 50 μL TMB substrate solution (Thermo) for detection. Optical density was measured at 450 nm on a BioRad xMark spectrophotometer. Washes were performed between each step using a BioRad ImmunoWash 1575. The normalized OD was calculated with the following equation: Mean Sample OD−(Mean Peptide OD+Mean Serum OD). To assess the performance of the peptide ELISA, ROC curves were generated with normalized OD thresholds ranging from −0.273 to 2.527. ELISA assay was performed on the PepSeq samples (51 confirmed CM patients and 55 blood bank controls) and the validation cohort, which included an independent CM sample set (n=18) and additional blood bank controls (n=30) that had not been assayed with the CWP PepSeq library. CM samples and non-endemic control samples were run side by side on the same 96-plate to avoid any batch effects. Each 96-well plate also included a positive control and two negative control samples to help identify any plate to plate variations.

### Immunodiffusion

Three blood bank samples demonstrated reactivity to >2 of the 31 peptides of interest identified in this study. To explore whether this reactivity could be explained by past fungal infections, these serum samples were independently tested for reactivity to Coccidioides spp., Histoplasma, Aspergillus and Blastomyces antigens using the IMMY ID Fungal Antibody System (ID1001) following the manufacturer's protocol. As internal controls for the assay, three CM patients from the validation cohort were also tested using the same approach and two were found to be positive for Coccidioides spp, and negative for Aspergillus. They were not run against Histoplasma or Blastomyces.

### Results

### Identification of Antibody Epitopes Commonly Targeted in CM Patients

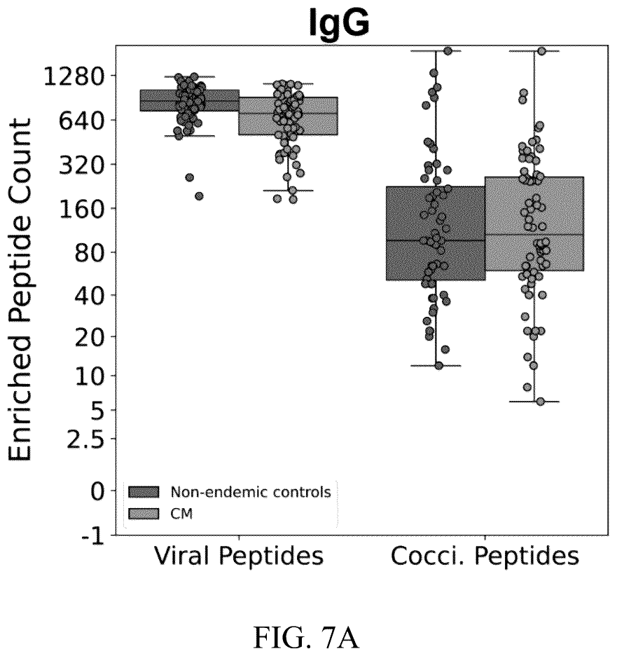

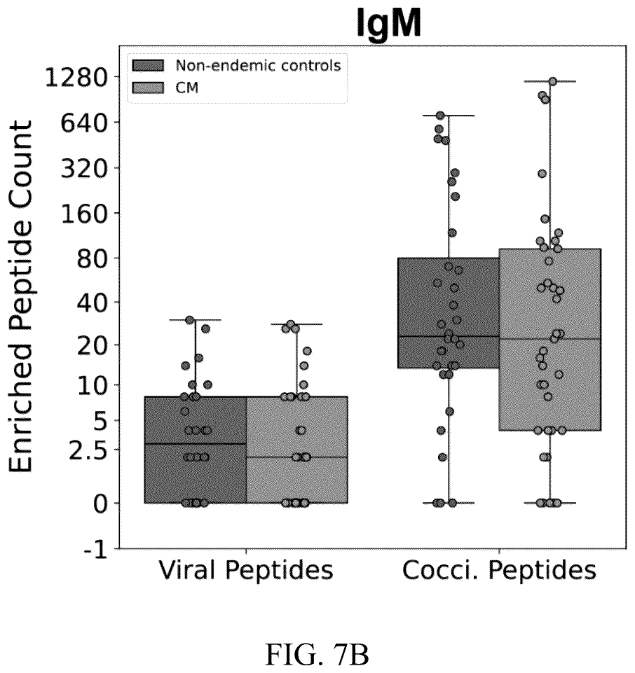

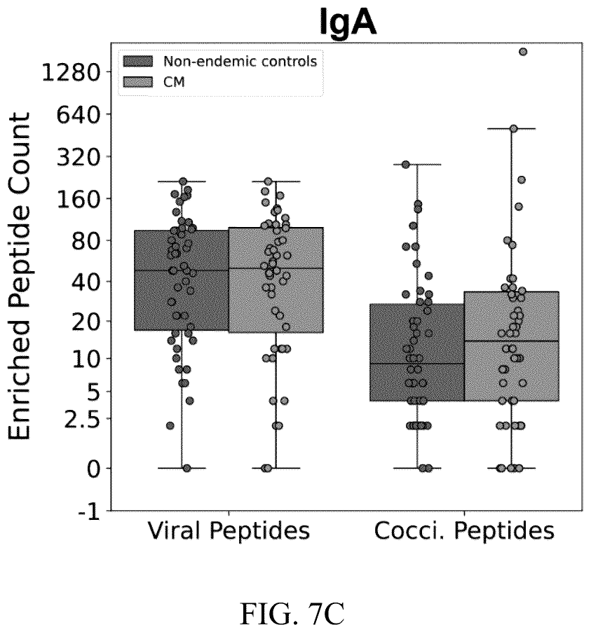

We used the CWP PepSeq library to measure the anti-Coccidioides spp. IgG antibody reactivity profiles in 70 CM patients and 60 control samples (healthy blood donors collected in Louisiana, USA-outside of the endemic range). Consistent with our expectations, both cohorts showed strong IgG antibody reactivity against a panel of 865 virus derived control peptides (93-628 peptides with Z≥10 in both replicates). However, on average, we observed higher numbers of enriched viral peptides in our non-endemic control samples compared to the CM samples (median=431.5 and 355.5, respectively; p=0.001, t-test) (FIG. 7A). In contrast, we observed similar numbers of Coccidioides spp. derived IgG enriched peptides in CM patients and non-endemic controls (median=54 and 49, respectively; pval=0.93, t-test) (FIG. 7A).

Due to variable levels of background for each individual peptide, we first used a dynamic thresholding approach where a threshold was calculated for each peptide. The threshold was set three standard deviations above the mean of the 97% highest density interval of the non-endemic control samples. Using these thresholds we calculated the number of positive and negative samples, and subsequently the positive predictive value (PPV) and sensitivity for all peptides in both the non-endemic and VF cohorts. We set a PPV threshold of 0.8 and a sensitivity threshold of 0.10. Using this approach we first identified 50 peptides that were elevated in CM (VF) patients. These peptides were designed from approximately 16 different proteins. As shown in Table 2, there were multiple peptide sequences that come from the same gene (Sequence Accession).

TABLE 2
Peptides elevated (enriched) in CM (VF) patient samples.

| | | Sequence | Reference | Start | Stop | SEQ ID |
|---|---|---|---|---|---|---|
| Peptide sequence | Species | Accession | Name | Index | Index | NO: |
| ATFSNFGSVLDIFAPGVDVESTMPGGGTQM | Coccidioides | KMP05693.1 | CWP_000547 | 308 | 338 | 1 |
| | immitis RMSCC | | | | | |
| | 2394 | | | | | |
| PTATLPPSPTGSACEPHGDHWHCEPLPTST | Coccidioides | TPX26831.1 | CWP_003724 | 132 | 162 | 2 |
| | immitis | | | | | |
| TGTCEPHGDHWHCPPGIPEPTTPPPSEPSI | Coccidioides | TPX26831.1 | CWP_003727 | 198 | 228 | 3 |
| | immitis | | | | | |
| PPPSEPSIPATTGTCEPHGDHWHCPPGVPE | Coccidioides | TPX26831.1 | CWP_003728 | 220 | 250 | 4 |
| | immitis | | | | | |
| KILEKPLAKSTGAKGKNKKKKKKPAKKQAE | Coccidioides | CPSG_03999 | CWP_007916 | 374 | 404 | 5 |
| | posadasii str. | | | | | |
| | Silveira | | | | | |
| ASPHHQPPVDKPKGGQEFYEAPKPEEPATT | Coccidioides | AOAOJ8S424 | CWP_020318 | | | 6 |
| | immitis H538.4 | | | | | |
| QRDYPTIDWEGLEDRIQAYFAELDQILTRK | Coccidioides | KMM70113.1 | CWP_022046 | 330 | 360 | 7 |
| | posadasii RMSCC | | | | | |
| | 3488 | | | | | |
| PKNPNPQPNPYDPHQPKRKPKPEPKPEPYD | Coccidioides | TPX21976.1 | CWP_028592 | 88 | 118 | 8 |
| | immitis | | | | | |
| EPKPEPYDPYWPKPNPYDPHQPKRKPKPEP | Coccidioides | TPX21976.1 | CWP_028593 | 110 | 140 | 9 |
| | immitis | | | | | |
| KRKPKPEPKPEPYDPYWPKPKPFDPFRPKP | Coccidioides | TPX21976.1 | CWP_028594 | 132 | 162 | 10 |
| | immitis | | | | | |
| FDPFRPKPKPYDPFRPESKPFDPFRPKPKP | Coccidioides | TPX21976.1 | CWP_028595 | 154 | 184 | 11 |
| | immitis | | | | | |
| RPKPKPYDPFRPEPKPYDPFRPESKPFDPF | Coccidioides | TPX21976.1 | CWP_028596 | 198 | 228 | 12 |
| | immitis | | | | | |
| ESKPFDPFRPESKPYDPFRPESKPYDPYWP | Coccidioides | TPX21976.1 | CWP_028597 | 220 | 250 | 13 |
| | immitis | | | | | |
| PYWPKPKPFDPYWPESKPKPKPKPRDPHQE | Coccidioides | TPX21976.1 | CWP_028598 | 286 | 316 | 14 |
| | immitis | | | | | |
| KPRDPHQETPQDPYQPKPQDPEPRPGDGDR | Coccidioides | TPX21976.1 | CWP_028599 | 308 | 338 | 15 |
| | immitis | | | | | |
| PRPGDGDRVINVGFPAGGLICHPACFPPSH | Coccidioides | TPX21976.1 | CWP_028600 | 330 | 360 | 16 |
| | immitis | | | | | |
| STCCELPKPKRESNPHDSDQPNPRPGDHDG | Coccidioides | TPX21976.1 | CWP_028602 | 374 | 404 | 17 |
| | immitis | | | | | |
| HPHHHPHDSHQPNPRPGDRDGPLDPNQPAP | Coccidioides | TPX21976.1 | CWP_028603 | 415 | 445 | 18 |
| | immitis | | | | | |
| RLLDDYDAHRADRSSGALSFAPKFDVRESK | Coccidioides | QVM06765.1 | CWP_037828 | 66 | 96 | 19 |
| | posadasii str. | | | | | |
| | Silveira | | | | | |
| YTSGTPPGKASEAAKGKETETGERYWVSER | Coccidioides | QVM06765.1 | CWP_037831 | 132 | 162 | 20 |
| | posadasii str. | | | | | |
| | Silveira | | | | | |
| VKEKEESVQQDTSKTGMEADNELDGSAIDG | Coccidioides | QVM12200.1 | CWP_039076 | 330 | 360 | 21 |
| | posadasii str. | | | | | |
| | Silveira | | | | | |
| ESPRGWWPDLQPKNPNPQPKPYDPYQPKPK | Coccidioides | KMP05713.1 | CWP_054479 | 66 | 96 | 22 |
| | immitis RMSCC | | | | | |
| | 2394 | | | | | |
| PESKPYDPYQPELKPFDPYQPESKPFDPYW | Coccidioides | KMP05713.1 | CWP_054480 | 154 | 184 | 23 |
| | immitis RMSCC | | | | | |
| | 2394 | | | | | |
| SKPFDPYWPESKPFDPYQPESKPFDPYQPE | Coccidioides | KMP05713.1 | CWP_054481 | 176 | 206 | 24 |
| | immitis RMSCC | | | | | |
| | 2394 | | | | | |
| PFDPYQPESKPFDPYWPESNHYDSYQLEPR | Coccidioides | KMP05713.1 | CWP_054482 | 198 | 228 | 25 |
| | immitis RMSCC | | | | | |
| | 2394 | | | | | |
| DSYQLEPRAGPRPFLRIPRPRPRPHRPRPH | Coccidioides | KMP05713.1 | CWP_054483 | 220 | 250 | 26 |
| | immitis RMSCC | | | | | |
| | 2394 | | | | | |
| GPHRPSRPQPRPQPVPIPQNPHLPNLDASL | Coccidioides | KMP05713.1 | CWP_054484 | 264 | 294 | 27 |
| | immitis RMSCC | | | | | |
| | 2394 | | | | | |
| LPNLDASLIDALLPKPHQETPQDPYQPKPQ | Coccidioides | KMP05713.1 | CWP_054485 | 286 | 316 | 28 |
| | immitis RMSCC | | | | | |
| | 2394 | | | | | |
| DPYQPKPQDPEPRPGDGDRVINVGFPAGDL | Coccidioides | KMP05713.1 | CWP_054486 | 308 | 338 | 29 |
| | immitis RMSCC | | | | | |
| | 2394 | | | | | |
| DLDCFTCCKPLKPKPKPESNPHDSDQPNPR | Coccidioides | KMP05713.1 | CWP_054491 | 418 | 448 | 30 |
| | immitis RMSCC | | | | | |
| | 2394 | | | | | |
| DSDQPNPRPGDHDGPIIDHGALDPNHPHHH | Coccidioides | KMP05713.1 | CWP_054492 | 440 | 470 | 31 |
| | immitis RMSCC | | | | | |
| | 2394 | | | | | |
| GDHDGPIIDHGALDPNHPHHHPHDSHQPAP | Coccidioides | KMP05713.1 | CWP_054493 | 449 | 479 | 32 |
| | immitis RMSCC | | | | | |
| | 2394 | | | | | |
| KPKPYDPHQPKPKPKPNPTSLIPSRLIRSI | Coccidioides | KMM71006.1 | CWP_054494 | | | 33 |
| | posadasii RMSCC | | | | | |
| | 3488 | | | | | |
| PRPFLRIPRPRPSPHRPRPHRPSRPSRPRP | Coccidioides | KMM71006.1 | CWP_054495 | | | 34 |
| | posadasii RMSCC | | | | | |
| | 3488 | | | | | |
| PTTSTTASQPTNGPPADECEPHGDHWHCPP | Coccidioides | KMP00029.1 | CWP_054709 | 198 | 228 | 35 |
| | immitis RMSCC | | | | | |
| | 2394 | | | | | |
| ASEFVTMAPRITLSDFPLLPLTYDDLNSMV | Coccidioides | TPX23161.1 | CWP_068503 | 1320 | 1350 | 36 |
| | immitis | | | | | |
| VTAGVPHLQQAGVNTPNLHPTTVGVLNELM | Coccidioides | TPX22474.1 | CWP_078535 | 858 | 888 | 37 |
| | immitis | | | | | |
| EHSYCDTYGCDGPMEPKTPKPTDCYGDCED | Coccidioides | QVM09276.1 | CWP_085538 | 66 | 96 | 38 |
| | posadasii str. | | | | | |
| | Silveira | | | | | |
| PPKKYGDCDDYDGYCDGPSKTSIKPEPPKP | Coccidioides | QVM09276.1 | CWP_085539 | 198 | 228 | 39 |
| | posadasii str. | | | | | |
| | Silveira | | | | | |
| MALSPTASCALACPEGDVCCQAACVGVPCP | Coccidioides | CPSG_07735 | CWP_088067 | 88 | 118 | 40 |
| | posadasii str. | | | | | |
| | Silveira | | | | | |
| CDQGNGSPAAIEAYAKCQSDCIKTYFFTGS | Coccidioides | CPSG_07735 | CWP_088069 | 132 | 162 | 41 |
| | posadasii str. | | | | | |
| | Silveira | | | | | |
| SDEEEEAEAEADKELSAKLSPNAADGKVEL | Coccidioides | TPX21583.1 | CWP_090285 | 374 | 404 | 42 |
| | immitis | | | | | |
| ATPSHYDEYGYKMRKRGAKEHSYCDTYGCD | Coccidioides | TPX21266.1 | CWP_103986 | 44 | 74 | 43 |
| | immitis | | | | | |
| YCDTYGCDGPMEPKPPKPTDCYGDCEDGYD | Coccidioides | TPX21266.1 | CWP_103987 | 66 | 96 | 44 |
| | immitis | | | | | |
| NEAYEAGLIGKNASGSGYDFDIYIHRGMGA | Coccidioides | KMM64979.1 | CWP_109300 | 220 | 250 | 45 |
| | posadasii RMSCC | | | | | |
| | 3488 | | | | | |
| KLENVPSPDFGLFSDILIGTIRSEIADSSE | Coccidioides | TPX22885.1 | CWP 189559 | 176 | 206 | 46 |
| | immitis | | | | | |
| SIAENAIKAEIMAKKDDDKHQHQGAKDFLV | Coccidioides | TPX23043.1+ | CWP_209904 | 22 | 52 | 47 |
| | immitis | A2:B48 | | | | |
| QSYDGGFAQAPWLEAHAGLTYSALGTLSFL | Coccidioides | QVM12318.1 | CWP_182912 | 198 | 228 | 48 |
| | posadasii str. | | | | | |
| | Silveira | | | | | |
| YPPHDCQSYGDDYGNCKGEKPSATPSHYDE | Coccidioides | TPX21266.1 | CWP_103985 | 22 | 52 | 49 |
| | immitis | | | | | |
| VDATEKFLIETAPGETRWVTEAEKFELKEK | Coccidioides | TPX21188.1 | CWP_196979 | 22 | 52 | 50 |
| | immitis | | | | | |

Of the first proteins that were identified only one has been previously identified as an antigenic protein, spherical outer wall glycoprotein (SOWgp). To our knowledge the other proteins of interest have not been previously studied. To better understand these proteins, we ran AA sequence-based prediction algorithms to predict signal peptides, transmembrane domains and cellular localization (deepLOC). The most common cellular localization for these proteins was extracellular, this is expected as extracellular and secreted proteins are more likely to generate an antibody response than intracellular proteins. The extracellular proteins contained both proteins expected to be secreted and lipid anchored. The other proteins were predicted to be localized to the cytoplasm (n=3), mitochondria (n=2), nucleus (n=2) and endoplasmic reticulum (n=1). All of which were predicted to either be soluble or lipid anchored. There was one protein which was predicted to have a transmembrane domain and it is expected to be localized to the endoplasmic reticulum.

To analyze the epitope level reactivity of the first identified proteins, we mapped the identified peptides to the protein sequences that they were designed from. Of the proteins we identified, several had multiple distinct reactive epitopes, with at least four proteins containing four or more epitopes (Table 2). For some proteins there are a small proportion of non-endemic control samples that show reactivity, potentially indicating cross reactivity with other fungal species. 31 IgG epitopes from five proteins had higher reactivity than non-endemic controls.

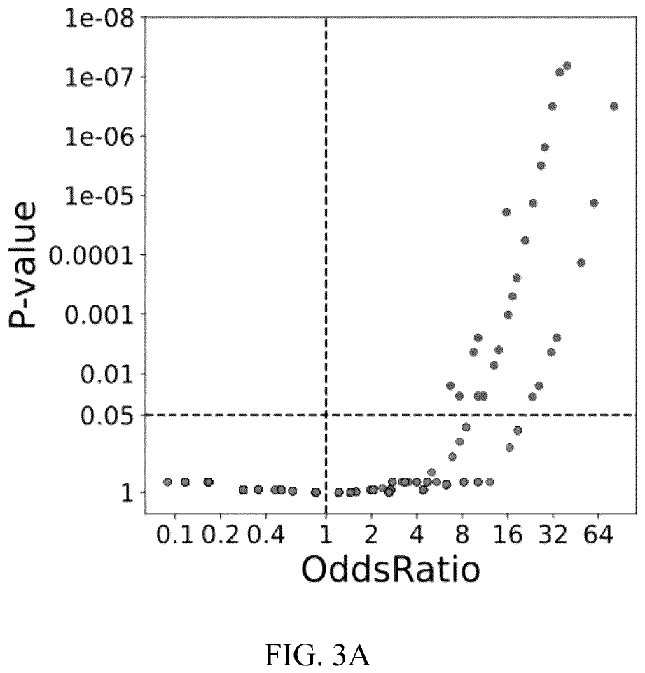

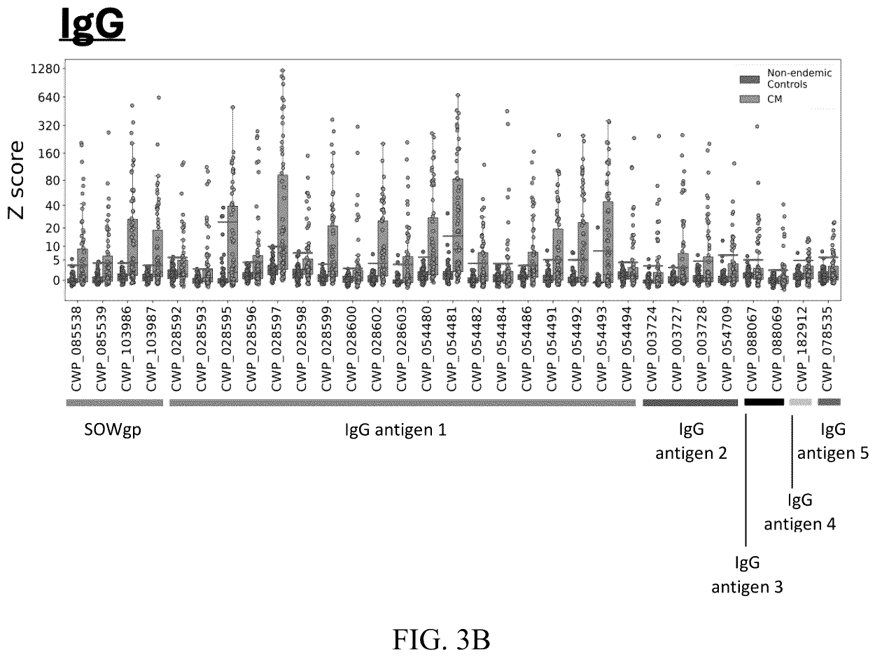

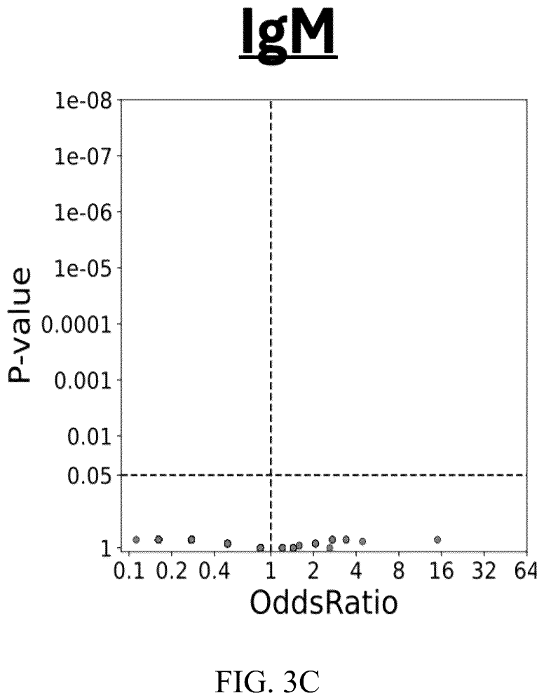

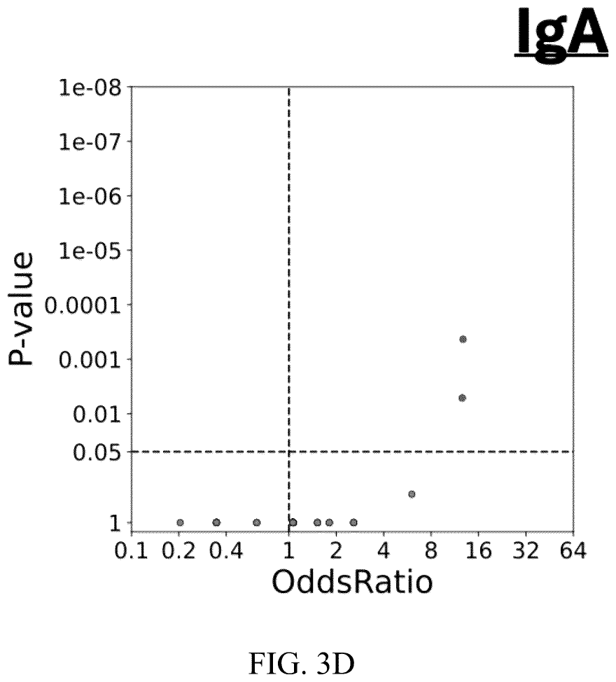

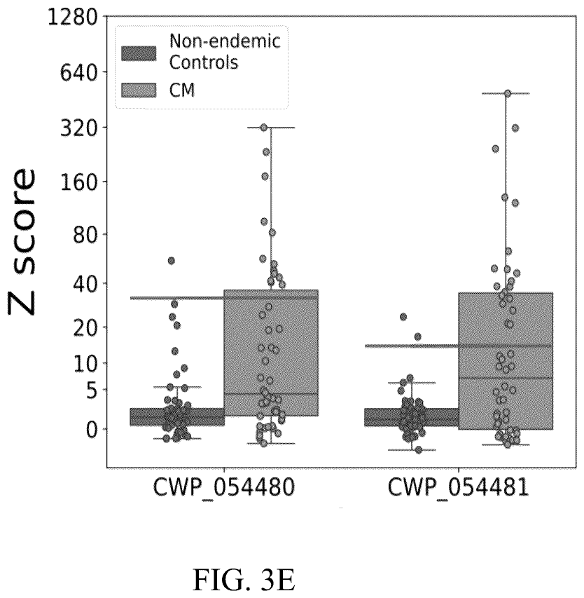

To exclude peptides with little to no evidence for antibody binding, we restricted our downstream analyses to peptides that had a Z score≥10 in at least 5 of the 130 total samples. For the IgG assay, 884 peptides met this threshold. From our human IgG reactivity profiles, we identified 31 peptides that were significantly enriched in the CM cohort compared to our controls, after multiple test correction (FIG. 3A, B; Table 3), with corrected p-values as low as 6.54×10⁻⁸. To assess the likelihood of false positives with our analysis approach, we randomly permuted the sample names, which resulted in an average of 0.042 significantly enriched peptides per permutation, with 99.6% of permutations having no significantly elevated peptides. We also ran a flipped analysis to determine whether any of the Coccidioides spp. derived peptides were significantly enriched in the control cohort and found zero elevated peptides.

TABLE 3
31 peptides significantly enriched in the CM cohort compared to controls (see, FIG. 3B).

| | | | | | | |
|---|---|---|---|---|---|---|
| | | | | | | SEQ |
| | | Sequence | Reference | Start | Stop | ID |
| Peptide sequence | Species | Accession | Name | Index | Index | NO: |

SOWgp

| | | | | | | |
|---|---|---|---|---|---|---|
| EHSYCDTYGCDGPMEPKTPKPTDCYGDCED | Coccidioides posadasii | QVM09276.1 | CWP_085538 | 66 | 96 | 38 |
| | str. Silveira | | | | | |
| PPKKYGDCDDYDGYCDGPSKTSIKPEPPKP | Coccidioides posadasii | QVM09276.1 | CWP_085539 | 198 | 228 | 39 |
| | str. Silveira | | | | | |
| ATPSHYDEYGYKMRKRGAKEHSYCDTYGCD | Coccidioides immitis | TPX21266.1 | CWP_103986 | 44 | 74 | 43 |
| YCDTYGCDGPMEPKPPKPTDCYGDCEDGYD | Coccidioides immitis | TPX21266.1 | CWP_103987 | 66 | 96 | 44 |

IgG antigen 1

| | | | | | | |
|---|---|---|---|---|---|---|
| PKNPNPQPNPYDPHQPKRKPKPEPKPEPYD | Coccidioides immitis | TPX21976.1 | CWP_028592 | 88 | 118 | 8 |
| EPKPEPYDPYWPKPNPYDPHQPKRKPKPEP | Coccidioides immitis | TPX21976.1 | CWP_028593 | 110 | 140 | 9 |
| FDPFRPKPKPYDPFRPESKPFDPFRPKPKP | Coccidioides immitis | TPX21976.1 | CWP_028595 | 154 | 184 | 11 |
| RPKPKPYDPFRPEPKPYDPFRPESKPFDPF | Coccidioides immitis | TPX21976.1 | CWP_028596 | 198 | 228 | 12 |
| ESKPFDPFRPESKPYDPFRPESKPYDPYWP | Coccidioides immitis | TPX21976.1 | CWP_028597 | 220 | 250 | 13 |
| PYWPKPKPFDPYWPESKPKPKPKPRDPHQE | Coccidioides immitis | TPX21976.1 | CWP_028598 | 286 | 316 | 14 |
| KPRDPHQETPQDPYQPKPQDPEPRPGDGDR | Coccidioides immitis | TPX21976.1 | CWP_028599 | 308 | 338 | 15 |
| PRPGDGDRVINVGFPAGGLICHPACFPPSH | Coccidioides immitis | TPX21976.1 | CWP_028600 | 330 | 360 | 16 |
| STCCELPKPKRESNPHDSDQPNPRPGDHDG | Coccidioides immitis | TPX21976.1 | CWP_028602 | 374 | 404 | 17 |
| HPHHHPHDSHQPNPRPGDRDGPLDPNQPAP | Coccidioides immitis | TPX21976.1 | CWP_028603 | 415 | 445 | 18 |
| PESKPYDPYQPELKPFDPYQPESKPFDPYW | Coccidioides immitis | KMP05713.1 | CWP_054480 | 154 | 184 | 23 |
| | RMSCC 2394 | | | | | |
| SKPFDPYWPESKPFDPYQPESKPFDPYQPE | Coccidioides immitis | KMP05713.1 | CWP_054481 | 176 | 206 | 24 |
| | RMSCC 2394 | | | | | |
| PFDPYQPESKPFDPYWPESNHYDSYQLEPR | Coccidioides immitis | KMP05713.1 | CWP_054482 | 198 | 228 | 25 |
| | RMSCC 2394 | | | | | |
| GPHRPSRPQPRPQPVPIPQNPHLPNLDASL | Coccidioides immitis | KMP05713.1 | CWP_054484 | 264 | 294 | 27 |
| | RMSCC 2394 | | | | | |
| DPYQPKPQDPEPRPGDGDRVINVGFPAGDL | Coccidioides immitis | KMP05713.1 | CWP_054486 | 308 | 338 | 29 |
| | RMSCC 2394 | | | | | |
| DLDCFTCCKPLKPKPKPESNPHDSDQPNPR | Coccidioides immitis | KMP05713.1 | CWP_054491 | 418 | 448 | 30 |
| | RMSCC 2394 | | | | | |
| DSDQPNPRPGDHDGPIIDHGALDPNHPHHH | Coccidioides immitis | KMP05713.1 | CWP_054492 | 440 | 470 | 31 |
| | RMSCC 2394 | | | | | |
| GDHDGPIIDHGALDPNHPHHHPHDSHQPAP | Coccidioides immitis | KMP05713.1 | CWP_054493 | 449 | 479 | 32 |
| | RMSCC 2394 | | | | | |
| KPKPYDPHQPKPKPKPNPTSLIPSRLIRSI | Coccidioides posadasii | KMM71006.1 | CWP_054494 | 133 | 162 | 33 |
| | RMSCC 3488 | | | | | |

IgG antigen 2

| | | | | | | |
|---|---|---|---|---|---|---|
| PTATLPPSPTGSACEPHGDHWHCEPLPTST | Coccidioides immitis | TPX26831.1 | CWP_003724 | 132 | 162 | 2 |
| TGTCEPHGDHWHCPPGIPEPTTPPPSEPSI | Coccidioides immitis | TPX26831.1 | CWP_003727 | 198 | 228 | 3 |
| PPPSEPSIPATTGTCEPHGDHWHCPPGVPE | Coccidioides immitis | TPX26831.1 | CWP_003728 | 220 | 250 | 4 |
| PTTSTTASQPTNGPPADECEPHGDHWHCPP | Coccidioides immitis | KMP00029.1 | CWP_054709 | 198 | 228 | 35 |
| | RMSCC 2394 | | | | | |

IgG antigen 3

| | | | | | | |
|---|---|---|---|---|---|---|
| MALSPTASCALACPEGDVCCQAACVGVPCP | Coccidioides posadasii | CPSG_07735 | CWP_088067 | 88 | 118 | 40 |
| | str. Silveira | | | | | |
| CDQGNGSPAAIEAYAKCQSDCIKTYFFTGS | Coccidioides posadasii | CPSG_07735 | CWP_088069 | 132 | 162 | 41 |
| | str. Silveira | | | | | |

IgG antigen 4

| | | | | | | |
|---|---|---|---|---|---|---|
| QSYDGGFAQAPWLEAHAGLTYSALGTLSFL | Coccidioides posadasii | QVM12318.1 | CWP_182912 | 198 | 228 | 48 |
| | str. Silveira | | | | | |

IgG antigen 5

| | | | | | | |
|---|---|---|---|---|---|---|
| VTAGVPHLQQAGVNTPNLHPTTVGVLNELM | Coccidioides immitis | TPX22474.1 | CWP_078535 | 858 | 888 | 37 |

Z score analysis showed that 87% of CM samples were reactive to at least one of the 31 identified peptides, and each sample, on average, was reactive to 11/31 peptides (35%) (FIG. 3B). These peptides were designed from six different proteins (Table 4), with four of the six proteins having multiple significantly elevated peptides. Four of the identified peptides were designed from the known CM antigen, SOWgp (CWP_085538, CWP_085539, CWP_103986, CWP_103987) (FIG. 3B). However, the remaining five proteins have not previously been described as immunoreactive. Strikingly, one of these five, which we have named IgG antigen 1, contained 19 significantly enriched peptides.

TABLE 4
Protein characteristics for the six significantly elevated proteins in CM patient samples.

| | Protein | | | Membrane | Transcriptome | |
|---|---|---|---|---|---|---|
| Protein Name | Accession | Localizations | Signals | types | Data | Potential annotation |
| SOWgp | TPX21266.1, | Extracellular | | Lipid anchor | upregulated in | sperule outer wall |
| | QVM09276.1 | | | \| Soluble | spherules | glycoprotein |
| IgG antigen 1 | KMP05713.1, | Extracellular | Signal peptide | Soluble | upregulated in | filamentous hemagglutinin |
| | KMM71006.1, | | | | spherules | |
| | TPX21976.1, | | | | | |
| IgG antigen 2 | KMP00029.1, | Extracellular, | Signal peptide, | Soluble, Lipid | upregulated in | hypothetical protein |
| | TPX26831.1 | Extracellular | none | anchor | spherules | |
| IgG antigen 3 | CPSG_07735 | Extracellular | | Lipid anchor | upregulated in | extracellular membrane |
| | | | | \| Soluble | spherules | protein CFEM domain- |
| | | | | | | containing protein |
| IgG antigen 4 | QVM12318.1 | Cytoplasm | | Soluble | stable expression | geranylgeranyltransferase |
| IgG antigen 5 | TPX22474.1 | Nucleus | Nuclear | Soluble | downregulated | Zn(2)-C6 fungal-type |
| | | | localization signal | | in spherules | domain |

Next, we used analogous experimental and analytical methods (only substituting a different isotype capture) to assess the IgM reactivity profiles on a subset of CM patients (n=46) and non-endemic controls (n=39). There were few enriched viral peptides detected with the IgM screen in both the control and CM cohorts (median=2 and 1, respectively), and there was no significant difference between the number of Coccidioides spp. derived enriched peptides between the cohorts (pval=0.54, t-test) (FIG. 7B). We observed a total of 273 peptides that passed our reactivity threshold (Z score≥10 in ≥5 samples) but found none of these were significantly enriched in the CM cohort after multiple test correction. (FIG. 3C).

Lastly, we analyzed the IgA reactivity profiles of a subset of CM patients (n=50) and non-endemic controls (n=53). On average we observed a lower number of total enriched peptides (Z≥10 in both replicates) in our IgA screen compared to IgG (IgA median=42, IgG median=475.5) (FIG. 7C). The number of IgA enriched viral control peptides was similar between control and CM samples (medians=25 and 24.5, respectively), however we did see a trend towards an increased number of enriched Coccidioides spp. derived peptides in the CM samples compared to controls (medians=8 and 5, respectively, p=0.11, t-test) (FIG. 7C). There were 32 peptides that showed reactivity in at least five samples, and two peptides that were significantly enriched in our CM cohort after multiple test correction. Both of these peptides were also detected as significantly enriched in the IgG screen and both were designed from the same protein, IgG antigen 1, the protein with the highest number of peptides with diagnostic potential in our IgG analysis (FIGS. 3D, E).

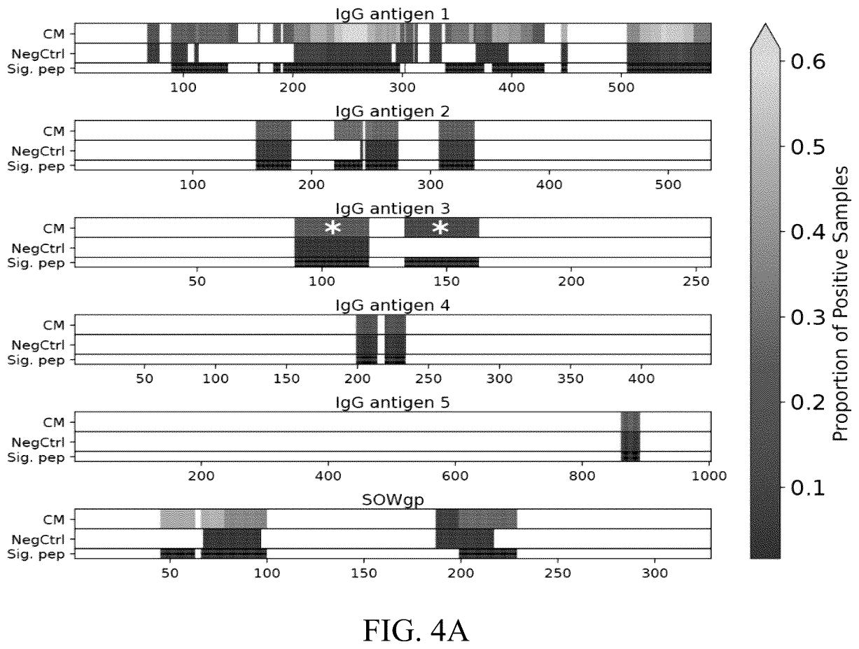

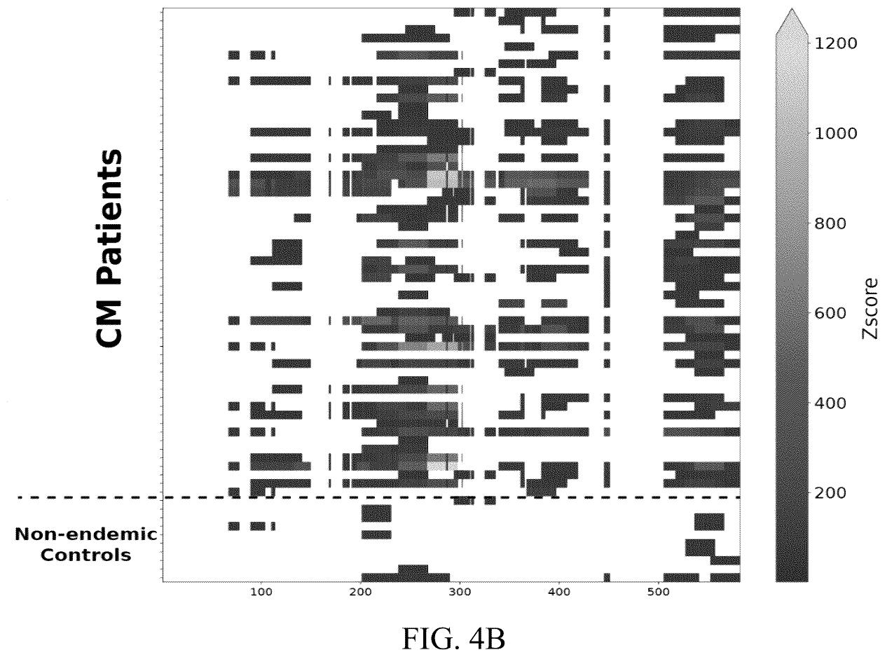

We then took the six identified proteins of interest and looked at the epitope level reactivity. The proportion of positive CM cases ranged from 0.10 to 0.63 across these epitopes (FIG. 4A). For some proteins there are a small proportion (0.02-0.10) of non-endemic control samples that show reactivity, potentially indicating cross reactivity with other pathogens. The protein with the highest amount of enriched peptides (IgG antigen 1) showed at least four epitopes that were broadly reactive across >35% of reactive CM samples, with the region from 200-300 showing the highest Z scores (FIG. 4B).

### Identification of Antibody Epitopes Commonly Targeted in Naturally Infected Pigtail Macaques

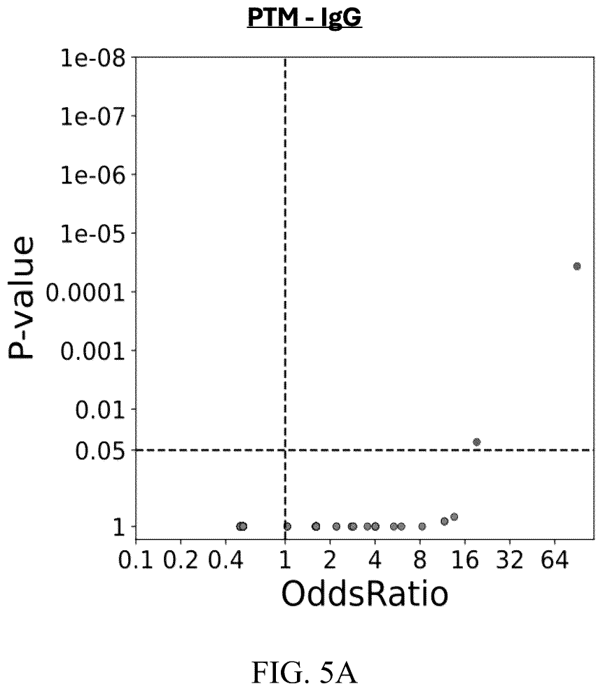

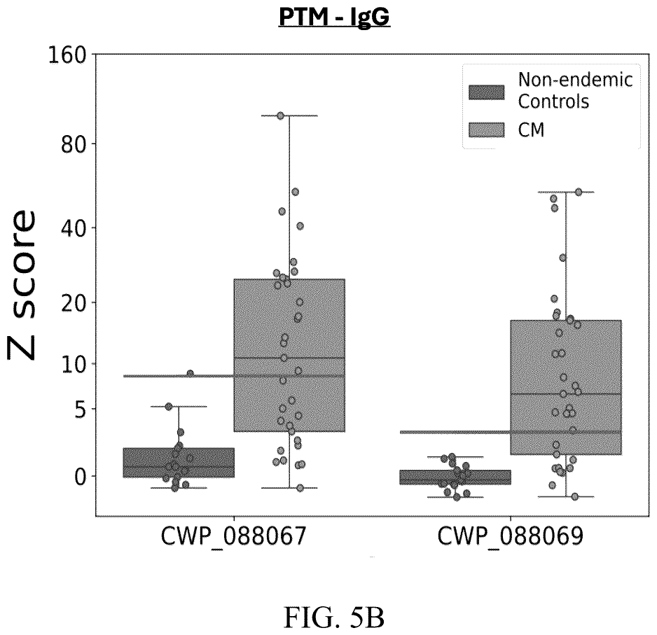

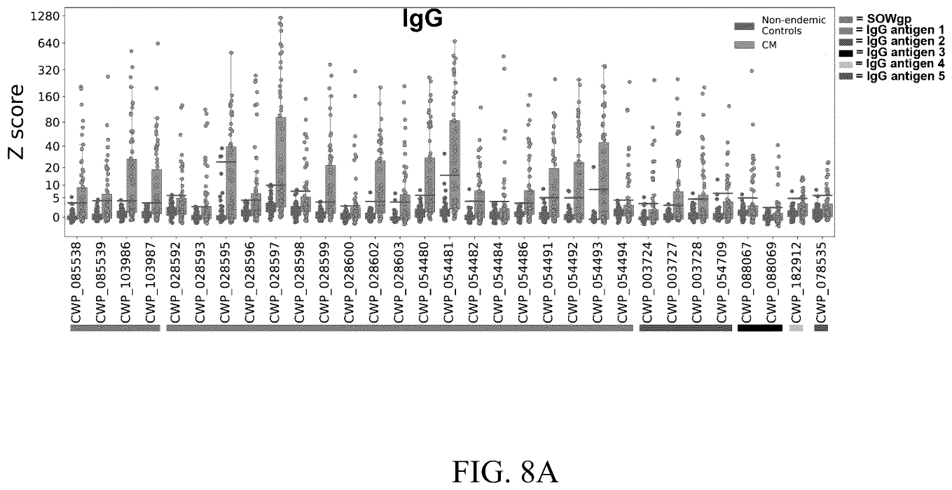

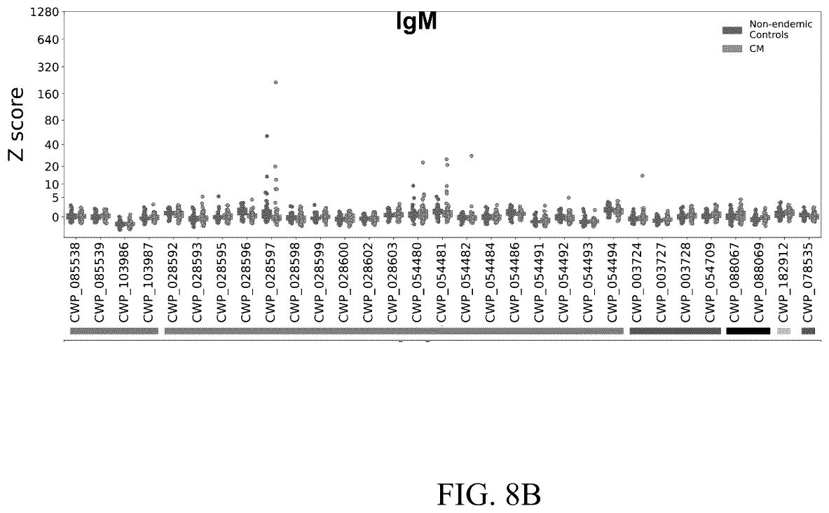

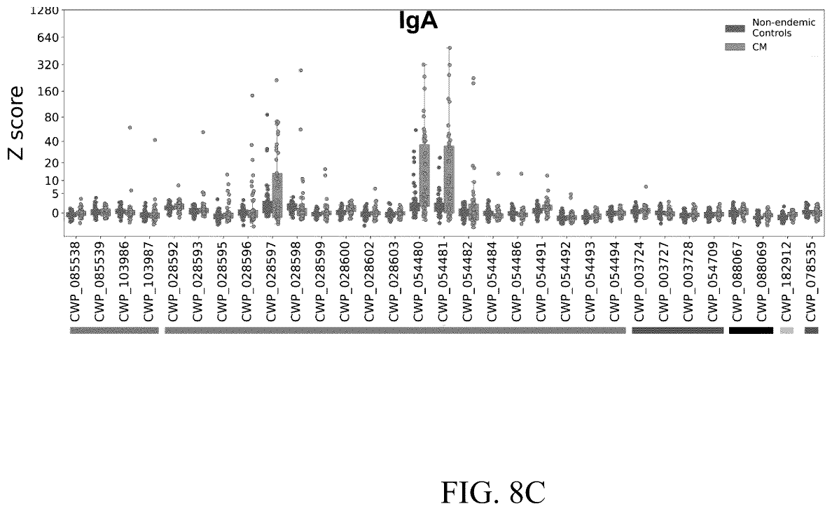

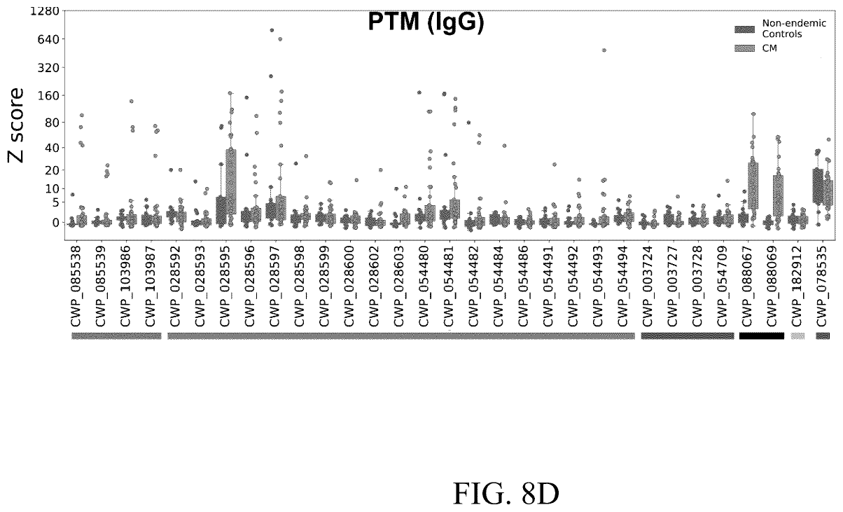

Pigtail macaques (PTM) living in a captive research colony in the endemic region (Mesa, AZ) can be naturally infected with Coccidioides spp³⁴. Previous characterization shows that these PTMs have similar disease manifestation as seen in humans³⁵. We took advantage of this collection of research samples to study the antibody response to Coccidioides spp. infection in a species that is likely to be important in future vaccine challenge studies. We measured the IgG reactivity profiles from PTMs with confirmed Coccidioides spp. infections (n=33) and control samples from PTMs housed in a separate colony outside of the endemic region (Seattle, WA) (n=17). We observed 104 peptides whose enrichment by IgG exceeded our threshold (Z score≥10) in at least 5 samples. We detected two significantly elevated peptides in the Coccidioides spp. infected PTM group, which were both designed from a single protein (IgG antigen 3) (FIG. 5A, B). Both peptides were also detected as significantly elevated in the human IgG analysis (FIG. 5B, FIG. 8).

### Characteristics of Immunogenic Proteins

Of the six proteins that were identified as showing significantly higher IgG reactivity in our CM cohort, only one has been previously identified as an antigenic protein: spherical outer wall glycoprotein (SOWgp). Interestingly, SOWgp and IgG antigen 1 appear to be unique to Coccidioides spp., while the other four proteins have moderate sequence similarity (53-73% identity, 50-100% query coverage) to other fungi. To better understand the biological functions and immune accessibility of the six proteins identified, we utilized the AA sequence based prediction algorithm, deepLOC 2.0, to predict the presence of signal peptides, transmembrane domains and cellular localization (Table 5) 29. Four of the six proteins (SOWgp, IgG antigens 1-3) were predicted to be localized extracellularly with an average model confidence of 0.83. IgG antigen 1 was predicted to contain a signal peptide and to be soluble (avg. confidence=0.90). Interestingly, the significantly enriched peptides for IgG antigen 2 were designed from domains that are shared between the two isoforms of this protein, one of which is predicted to have a signal peptide, whereas the other isoform was predicted to be lipid anchored (model confidence=0.84 and 0.96 respectively). SOWgp and IgG antigen 3 were both predicted to be lipid anchored with high confidence (0.94 and 0.98, respectively). IgG antigen 4 and IgG antigen 5 were predicted to be soluble (model confidence=0.69 and 0.75, respectively), however, IgG antigen 4 was predicted to be localized to the cytoplasm (model confidence=0.77) and IgG antigen 5 to the nucleus (model confidence=0.77) (Table 5). Additionally, transcriptional evidence suggests that four of the six proteins (SOWgp, IgG antigens 1-3) have increased gene expression during the host specific lifecycle (Table 4) 30,36.

TABLE 5
DeepLOC 2.0 predictions of subcellular localizations and the associated membrane types for six proteins of interest detected in human IgG screen

| | Protein | | | Membrane | | | | Cell | |
|---|---|---|---|---|---|---|---|---|---|
| Protein_ID | Accession | Localizations | Signals | types | Cytoplasm | Nucleus | Extracellular | membrane | Mitochondrion |
| SOWgp | TPX21266.1 | Extracellular | | Lipid anchor \| | 0.139 | 0.082 | 0.845 | 0.200 | 0.064 |
| | | | | Soluble | | | | | |
| SOWgp | QVM09276.1 | Extracellular | | Lipid anchor \| | 0.134 | 0.089 | 0.833 | 0.191 | 0.056 |
| | | | | Soluble | | | | | |
| IgG antigen 1 | KMP05713.1 | Extracellular | Signal peptide | Soluble | 0.253 | 0.119 | 0.806 | 0.079 | 0.056 |
| IgG antigen 1 | KMM71006.1 | Extracellular | Signal peptide | Soluble | 0.227 | 0.097 | 0.837 | 0.068 | 0.054 |
| IgG antigen 1 | TPX21976.1 | Extracellular | Signal peptide | Soluble | 0.207 | 0.101 | 0.925 | 0.062 | 0.058 |
| IgG antigen 2 | KMP00029.1 | Extracellular | Signal peptide | Soluble | 0.121 | 0.163 | 0.834 | 0.096 | 0.045 |
| IgG antigen 2 | TPX26831.1 | Extracellular | | Lipid anchor | 0.116 | 0.094 | 0.756 | 0.303 | 0.070 |
| IgG antigen 3 | CPSG_07735 | Extracellular | | Lipid anchor \| | 0.182 | 0.122 | 0.825 | 0.321 | 0.096 |
| | | | | Soluble | | | | | |
| IgG antigen 4 | QVM12318.1 | Cytoplasm | | Soluble | 0.770 | 0.476 | 0.046 | 0.066 | 0.131 |
| IgG antigen 5 | TPX22474.1 | Nucleus | Nuclear | Soluble | 0.446 | 0.765 | 0.034 | 0.069 | 0.125 |
| | | | localization signal | | | | | | |

| | | Endoplasmic | Lysosome/ | Golgi | | | | Lipid | |
|---|---|---|---|---|---|---|---|---|---|
| Protein_ID | Plastid | reticulum | Vacuole | apparatus | Peroxisome | Peripheral | Transmembrane | anchor | Soluble |
| SOWgp | 0.016 | 0.141 | 0.075 | 0.098 | 0.008 | 0.120 | 0.123 | 0.942 | 0.597 |
| SOWgp | 0.014 | 0.139 | 0.080 | 0.095 | 0.007 | 0.136 | 0.111 | 0.946 | 0.595 |
| IgG antigen 1 | 0.025 | 0.042 | 0.078 | 0.030 | 0.002 | 0.163 | 0.050 | 0.084 | 0.903 |
| IgG antigen 1 | 0.028 | 0.052 | 0.084 | 0.032 | 0.001 | 0.172 | 0.062 | 0.083 | 0.883 |
| IgG antigen 1 | 0.014 | 0.056 | 0.062 | 0.023 | 0.001 | 0.170 | 0.032 | 0.152 | 0.922 |
| IgG antigen 2 | 0.013 | 0.073 | 0.210 | 0.103 | 0.007 | 0.252 | 0.097 | 0.373 | 0.842 |
| IgG antigen 2 | 0.006 | 0.185 | 0.214 | 0.175 | 0.005 | 0.121 | 0.168 | 0.959 | 0.475 |
| IgG antigen 3 | 0.004 | 0.152 | 0.164 | 0.150 | 0.009 | 0.100 | 0.134 | 0.987 | 0.553 |
| IgG antigen 4 | 0.000 | 0.302 | 0.166 | 0.255 | 0.128 | 0.327 | 0.106 | 0.048 | 0.688 |
| IgG antigen 5 | 0.010 | 0.174 | 0.230 | 0.125 | 0.024 | 0.333 | 0.183 | 0.083 | 0.747 |

### Diagnostic Potential of Identified Peptides

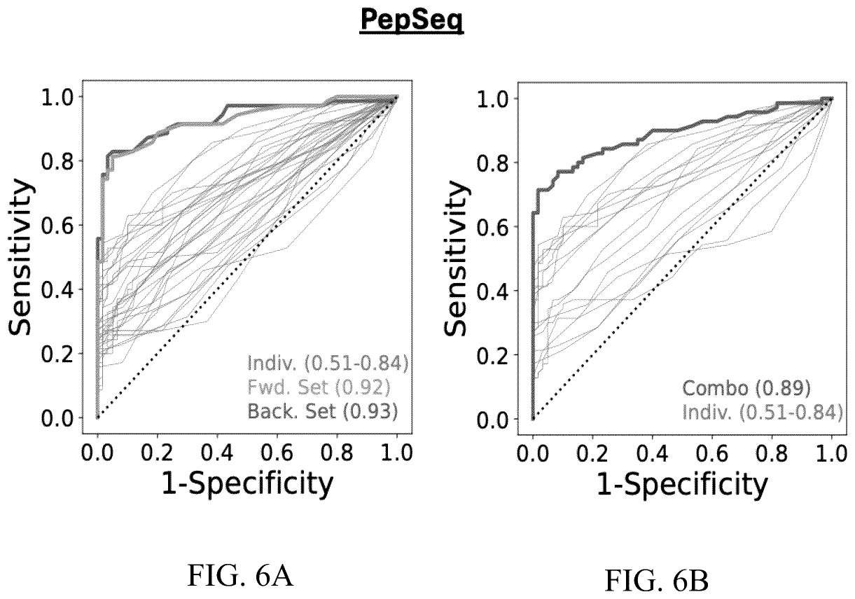

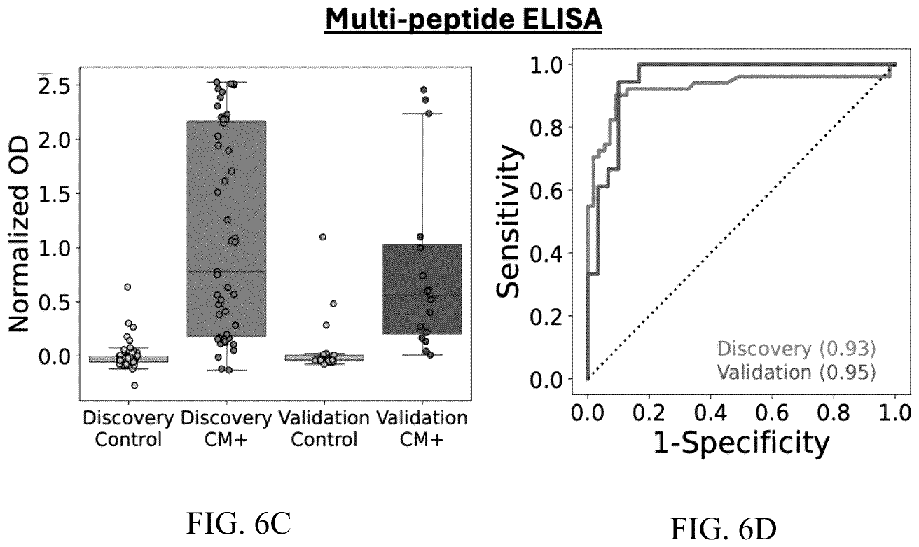

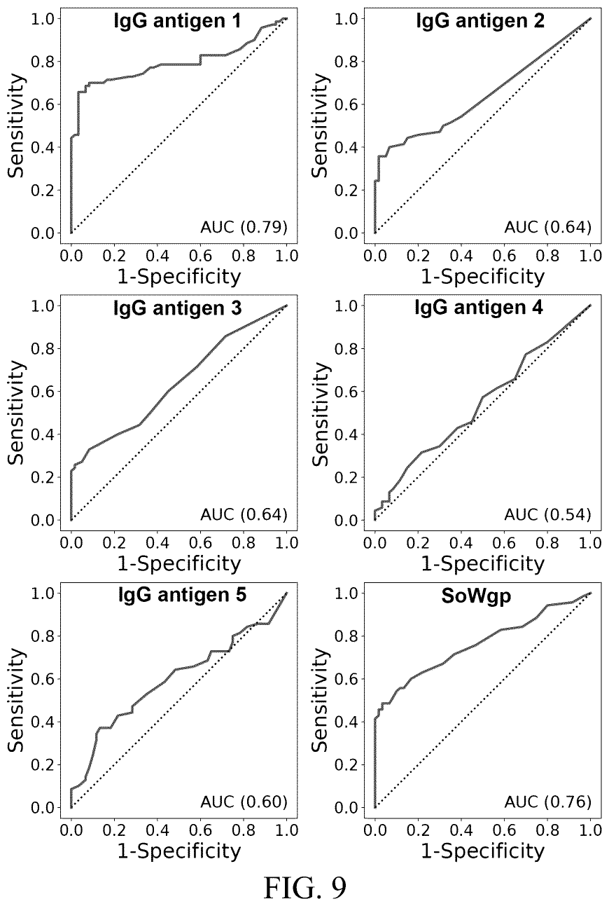

To assess the diagnostic potential of the immunogenic peptides within our discovery cohort, we generated receiver operating characteristic (ROC) curves. The area under the curve (AUC) for the 31 IgG peptides of interest individually ranged from 0.51 to 0.84 (FIG. 6A). We then looked at the protein level diagnostic potential of the identified proteins by taking the sum of the Z scores for all peptides designed from a protein. This analysis resulted in AUCs ranging from 0.54 to 0.79 with IgG antigen 1 having the highest AUC (FIG. 9). Finally, we used both forward and backward selection approaches to identify combinations of peptides with maximum diagnostic potential. This analysis identified sets of four and nine peptides, respectively, with all four of the peptides selected by forward selection contained in the set of peptides selected by backward selection. Both sets contained peptides derived from SOWgp and IgG antigen 1, while the reverse selection set also included peptides from IgG antigen 2 and IgG antigen 3. The sum of the Z scores for these selected peptides resulted in AUCs of 0.92 for the forward selection set and 0.93 for the backward selection set (FIG. 6A).

### ELISA

To validate the results from our highly-multiplexed assay and assess the diagnostic potential of the identified peptides in a simpler assay format, we created a multipeptide ELISA and used it to measure IgG antibody responses within a subset of our discovery cohort, as well as a validation cohort (samples not assayed with PepSeq). Using preliminary data from an initial subset of our final dataset (n=101), we chose 16 peptides (from seven proteins) to include in our multipeptide ELISA (Table 6). The 16 peptides from Table 2 were selected based on Z score reactivity to use in an ELISA assay. The peptides in Table 6 were synthesized with biotin on C-terminus, and subsequently assayed. The peptides can alternatively be modified with biotin on N-terminus, rather than on the C-terminus. Protein information for the proteins is provided in Table 7.

TABLE 6
Peptides in multipeptide ELISA

| Peptide_ | Peptide_Sequence | | | SEQ ID |
|---|---|---|---|---|
| Name | (c_term_modified) | Purity | Protein | NO: |
| CWP_000547 | ATFSNFGSVLDIFAPGVDVESTMPGGGTQM | 83.00% | KMP05693.1 | 1 |
| CWP_003727 | TGTCEPHGDHWHCPPGIPEPTTPPPSEPSI | 97.00% | TPX26831.1 | 3 |
| CWP_022046 | QRDYPTIDWEGLEDRIQAYFAELDQILTRK | 93.00% | KMM70113.1 | 7 |
| CWP_028597 | ESKPFDPFRPESKPYDPFRPESKPYDPYWP | 58.00% | TPX21976.1 | 13 |
| CWP_028599 | KPRDPHQETPQDPYQPKPQDPEPRPGDGDR | 97.00% | TPX21976.1 | 15 |
| CWP_028602 | STCCELPKPKRESNPHDSDQPNPRPGDHDG | 95.00% | TPX21976.1 | 17 |
| CWP_054481 | SKPFDPYWPESKPFDPYQPESKPFDPYQPE | 97.00% | KMP05713.1 | 24 |
| CWP_054483 | DSYQLEPRAGPRPFLRIPRPRPRPHRPRPH | 96.00% | KMP05713.1 | 26 |
| CWP_054486 | DPYQPKPQDPEPRPGDGDRVINVGFPAGDL | 92.00% | KMP05713.1 | 29 |
| CWP_054493 | GDHDGPIIDHGALDPNHPHHHPHDSHQPAP | 97.00% | KMP05713.1 | 32 |
| CWP_068503 | ASEFVTMAPRITLSDFPLLPLTYDDLNSMV | 96.00% | TPX23161.1 | 36 |
| CWP_085538 | EHSYCDTYGCDGPMEPKTPKPTDCYGDCED | 86.00% | QVM09276.1 | 38 |
| CWP_085539 | PPKKYGDCDDYDGYCDGPSKTSIKPEPPKP | 69.00% | QVM09276.1 | 39 |
| CWP_088067 | MALSPTASCALACPEGDVCCQAACVGVPCP | 69.00% | CPSG_07735 | 40 |
| CWP_103986 | ATPSHYDEYGYKMRKRGAKEHSYCDTYGCD | 70.00% | TPX21266.1 | 43 |
| CWP_103987 | YCDTYGCDGPMEPKPPKPTDCYGDCEDGYD | 95.00% | TPX21266.1 | 44 |

TABLE 7
Protein Information for peptides in the ELISA

| Protein | | Signal | Trans- | Protein | Transcript | | |
|---|---|---|---|---|---|---|---|
| Accession. | FungiDB ID | Peptide | membrane | Length (AA) | Length (nt) | Other Names | Transcript data |
| TPX21266.1 | DIZ76_015222 | Yes | No | 230 | 693 | SOWgp | up in spherules |
| TPX21976.1 | DIZ76_015941 | Yes | No | 445 | 1,338 | | up in spherules |
| KMP05713.1 | | Yes | No | 504 | 1,792 | Filamentous | |
| | | | | | | hemagglutinin | |
| QVM09276.1 | D8B26_003939 | Yes | No | 328 | 1,053 | Carbohydrate-binding | up in spherules |
| | | | | | | protein | |
| TPX26831.1 | DIZ76_012294 | Yes | No | 466 | 1,401 | | |
| TPX23161.1 | DIZ76_012486 | No | No | 8,030 | 24,093 | Nonribosomal Peptide | down in |
| | | | | | | Synthase | spherules |
| KMM70113.1 | CPAG_06426 | No | No | 493 | 1,896 | | |
| CPSG_07735 | CPSG_07735 | No | Yes | 252 | 2,154 | HFB protein | up in spherules |
| KMM71006.1 | CPAG_07313 | Yes | No | 460 | 1,383 | Filamentous | up in WT down |
| | | | | | | hemagglutinin/ZN(2) | in csp1 mutant |
| | | | | | | C6- fungal type domain | |
| KMP05693.1 | | Yes | No | 397 | 1,194 | subtilisin-like protease | |

To determine the diagnostic potential of this subset of 16 peptides using PepSeq, we generated ROC curves for each peptide individually and combined, which resulted in a combined AUC of 0.89 (FIG. 6B). From our discovery cohort, we randomly selected 51 CM and 55 non-endemic control samples to be assayed using the multipeptide ELISA. Additionally, we ran an independent validation cohort of 18 CM positive samples and 30 non-endemic control samples that had not been previously assayed with PepSeq and therefore were not involved in the selection of the peptides. The CM positive samples had significantly higher ODs than the controls in both the discovery and validation cohorts (Control-discovery mean=0.0009, CM-discovery mean=1.11, t-test pval=2.85×10⁻¹⁴) (Control-validation mean=0.022 CM-validation mean=0.98, pval=2.35×10⁻⁶) (FIG. 6C). ROC curves were used to assess the diagnostic potential of these peptides. AUCs were 0.93 for the discovery cohort, 0.95 for the validation cohort (FIG. 6D) demonstrating the diagnostic potential of the new targets. When setting an optimal OD threshold based on the discovery cohort data (OD=0.092), the sensitivity and specificity were 89% and 90% for the validation cohort.

### Detection of Fungal Antibodies Using a Commercial Assay

In the human IgG PepSeq assay, there were three samples in the discovery non-endemic control group that demonstrated reactivity to two or more of the 31 CM IgG enriched peptides. To look for evidence of a past fungal infection, we tested the serum from these samples, along with three CM positives that showed high levels of IgG reactivity in our PepSeq assay, using a commercial assay designed to detect antibody reactivity to Coccidioides, Histoplasma, Aspergillus and Blastomyces antigens. None of the discovery non-endemic control samples had detectable reactivity to any antigens tested and two out of three CM validation positives were reactive to the Coccidioides spp. antigen only. In addition, the three CM discovery patients with high titers but low anti-Coccidioides spp. PepSeq reactivity were run against the Coccidioides spp. antigen, two out of the three were positive (Table 8). In the ELISA assay, there were three validation controls that had slightly elevated OD's. Because of volume limitations only two were assayed against the Coccidioides spp. antigen, both of which were negative.

TABLE 8
Immunodiffusion results

| Sample ID | Cohort | Coccidioides Ag | Aspergillus Ag | Histoplasma Ag | Blastomyces Ag | Reason for testing |
|---|---|---|---|---|---|---|
| S00415 | Discovery | Negative | Negative | Negative | Negative | Some reactivity on PepSeq assay |
| | Control | | | | | |
| S00288 | Discovery | Negative | Negative | Negative | Negative | Some reactivity on PepSeq assay |
| | Control | | | | | |
| S00416 | Discovery | Negative | Negative | Negative | Negative | Some reactivity on PepSeq assay |
| | Control | | | | | |
| S00411 | Discovery | Negative | Negative | Negative | Negative | Some reactivity on PepSeq assay |
| | Control | | | | | |
| S00229 | Discovery | Negative | Negative | Negative | Negative | Low/none reactivity on PepSeq |
| | Control | | | | | assay |
| S00361 | Discovery | Negative | Negative | Negative | Negative | Low/none reactivity on PepSeq |
| | Control | | | | | assay |
| S00281 | Discovery | Negative | Negative | Negative | Negative | Low/none reactivity on PepSeq |
| | Control | | | | | assay |
| S00260 | Discovery | Negative | Negative | Negative | Negative | Low/none reactivity on PepSeq |
| | Control | | | | | assay |
| S00306 | Discovery | Negative | Negative | Negative | Negative | Low/none reactivity on PepSeq |
| | Control | | | | | assay |
| S00346 | Validation | Negative | Not tested | Not tested | Not tested | Slightly elevated OD on ELISA |
| | Control | | | | | |
| S00225 | Validation | Negative | Not tested | Not tested | Not tested | Slightly elevated OD on ELISA |
| | Control | | | | | |
| TGen05 | CM | Negative | Negative | Not tested | Not tested | Known positive |
| | Validation | | | | | |
| TGen15 | CM | Positive | Negative | Not tested | Not tested | Known positive |
| | Validation | | | | | |
| TGen17 | CM | Positive | Negative | Not tested | Not tested | Known positive |
| | Validation | | | | | |
| ADC2027 | CM | Negative | Not tested | Not tested | Not tested | High titer, low PepSeq score |
| | Discovery | | | | | |
| ADC2028 | CM | Positive | Not tested | Not tested | Not tested | High titer, low PepSeq score |
| | Discovery | | | | | |
| ADC2003 | CM | Positive | Not tested | Not tested | Not tested | High titer, low PepSeq score |
| | Discovery | | | | | |

### Discussion

In this study, we utilized highly-multiplexed serology to identify novel antigens from across the Coccidioides spp. proteome. Using a 244,000-plex Coccidioides specific PepSeq library (CWP), we probed CM positive and control sera from humans and PTMs to determine reactivity profiles for IgG (humans and PTM), IgM (humans) and IgA (humans). Although we saw no significant difference in overall numbers of enriched Coccidioides spp. derived peptides in the IgG, IgA or IgM screens, when applying peptide-specific comparisons we detected a strong signal in the IgG screen that robustly differentiated CM and non-endemic control samples. The high number of enriched Coccidioides spp. derived peptides in the non-endemic control cohort are likely due to the presence of cross reactive antibodies generated in response to exposures/infections with other microorganisms (FIG. 7).

In humans, we identified two peptides with significantly increased IgA reactivity in CM patients compared to non-endemic controls (FIG. 3D, E). Interestingly, while not significant with our current sample size, we do observe a small number of CM samples showing elevated reactivity to 16 of the peptides identified in the IgG screen (FIG. 8C). These results suggest that humans produce circulating IgA antibodies against Coccidioides spp. antigens. Comparing the IgG and IgA reactivity profiles for the two significant peptides (CWP_054480, CWP_054481), there appears to be less specificity with IgA compared to IgG. We looked at circulating IgA antibodies in this study; however, future investigations of IgA antibodies from CM bronchoalveolar lavage fluid where this isotope is anticipated to be more abundant are underway39. We did not identify any peptides with significantly elevated IgM reactivity in our CM cohort and only observed modest reactivity in 1-2 samples to six of the peptides of interest determined from our IgG screen (FIG. 8B). This is not particularly surprising, given that IgM is typically only produced for the first two weeks of infection2,5, while the samples for which we have metadata (54/70 CM samples) and were characterized in this study were collected 0-3,388 days (mean=381 days) after diagnosis. Diagnosis usually occurs weeks after infection, making it unlikely that samples in the current study were collected in the first two weeks of infection⁴⁰. The ideal sample set to probe for novel IgM antigens would be collected during the first two weeks of infection. Therefore, in this example we primarily focused on antigens recognized by IgG antibodies, as IgG is the primary antibody isotype found circulating in blood and tends to be long lived⁴¹.

Using this approach, we identified increased IgG reactivity to 50 peptides from approximately 15-20 different proteins in the disease patients compared to the controls. IgG antibodies are the most characterized among Coccidioidomycosis patients, in addition to utilizing IgG as a diagnostic target the change in isotype abundance during disease can indicate recovery or increasing infection. The role in circulating IgA antibodies is not defined in the context of Coccidioidomycosis, IgA and IgG antibodies against Candida cell surface glycoprotein Als3p antigens are protective (Richardson, J. P. & Moyes, D. L. Adaptive immune responses to Candida albicans infection. Virulence 6, 327-337 (2015); Spellberg, B. et al. Antibody titer threshold predicts anti-candidal vaccine efficacy even though the mechanism of protection is induction of cell-mediated immunity. J. Infect. Dis. 197, 967-971 (2008)).

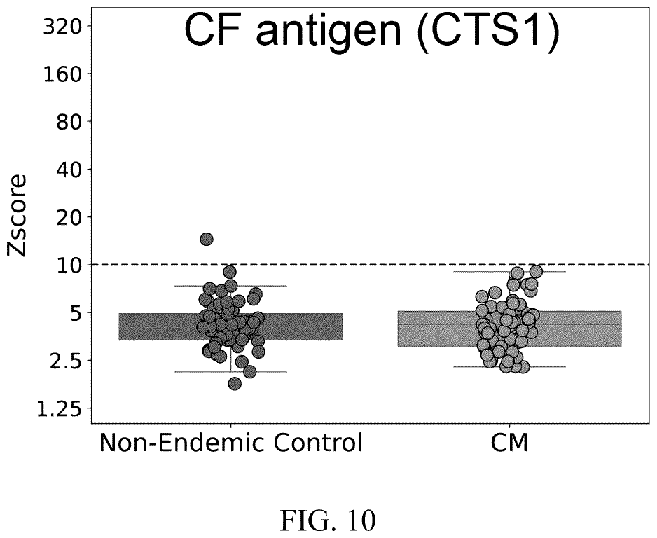

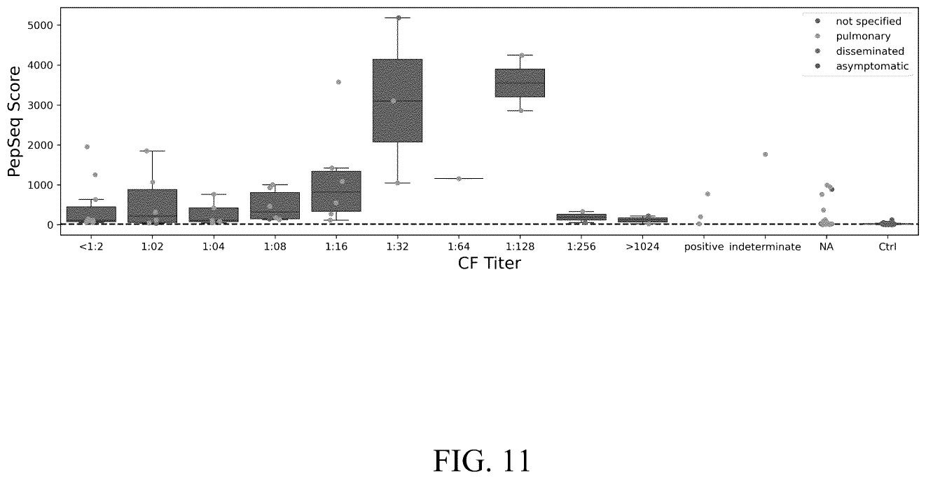

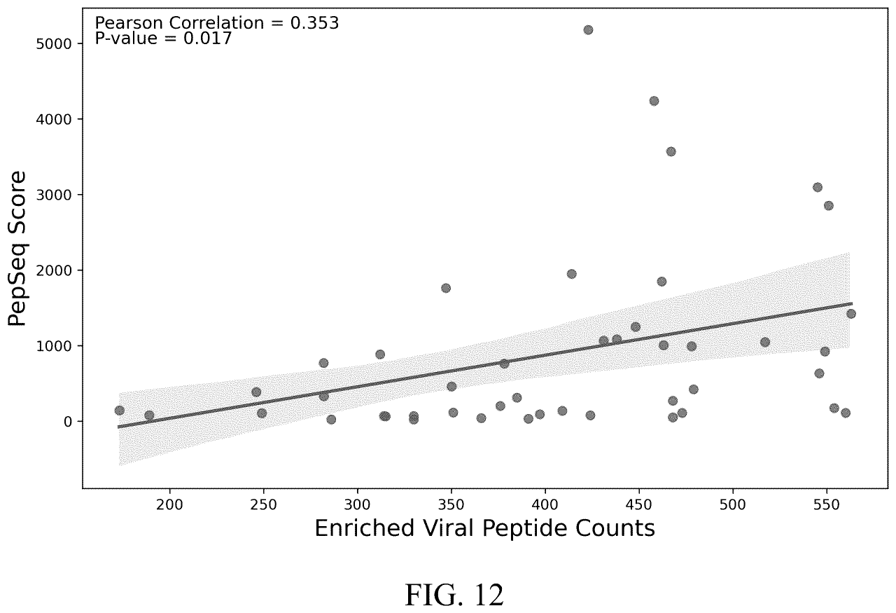

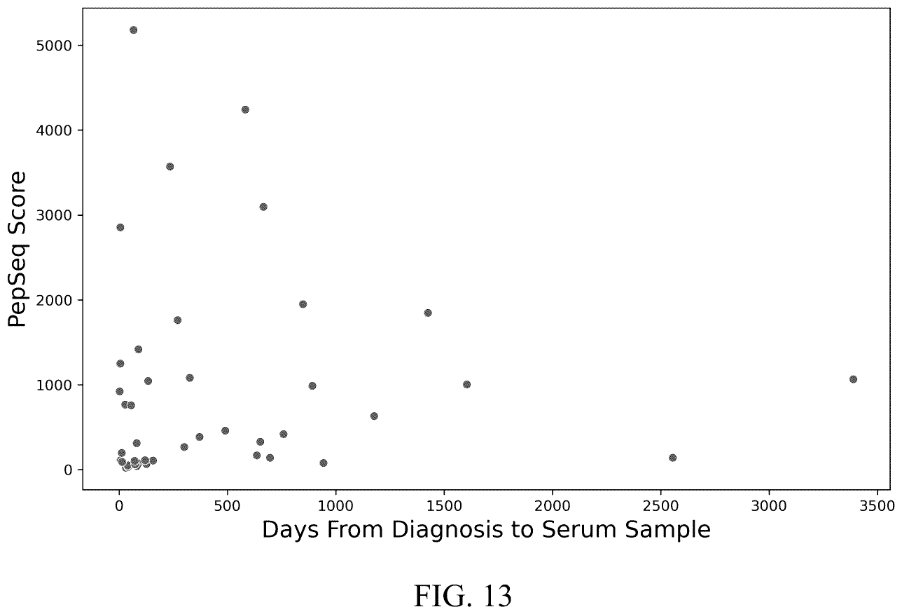

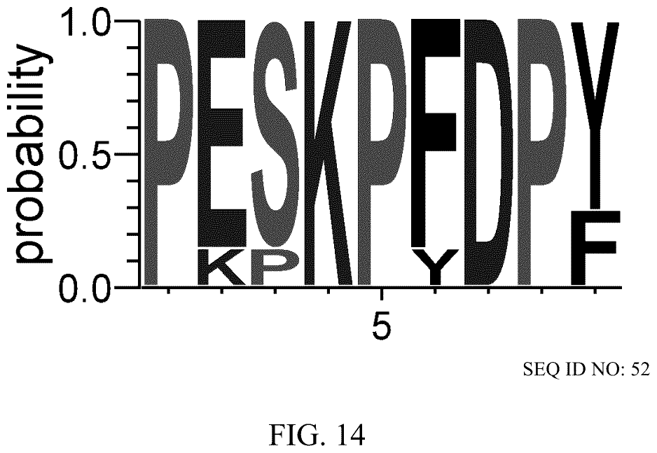

Further, we identified increased IgG reactivity to 31 peptides from six different proteins in CM patients compared to non-endemic controls, two of which overlapped with the significant peptides from the PTM samples and two others which showed significantly elevated IgA reactivity. A surprising result of our analysis was the lack of reactivity to any of the 45 peptides covering the current diagnostic IgG target, CF antigen, for which at least 48 CM patients had a positive titer (for 22 CM patients CF titer information was not available) (FIG. 10, 11, Table 9). One limitation of our approach is that the 30 AA peptides can only accurately represent linear epitopes. It is possible that the antibodies that bind to CF antigen rely on specific tertiary or quaternary structures that are not represented by our peptides. However, a GLM analysis showed a significant positive correlation between the summed Z score of our 31 IgG peptides of interest and CF titer, when controlling for gender, diagnosis, sample location, year of diagnosis and viral peptide counts (estimation of sample quality). We also observed a significant positive correlation between reactive viral peptide counts and the summed Z scores, potentially indicating that there are differences in overall sample reactivity/quality within our CM cohort, which may help explain why some of our CM discovery samples exhibit low reactivity against our 31 peptides of interest (Pearson correlation=0.353, pval=0.017) (FIG. 12). Notably, the four samples with the highest CF titers (1:256 and >1:1024) showed very low PepSeq Z scores (FIG. 11). It is possible these patients targeted a different set of epitopes, some of which are conformation specific, but more samples are needed at these high CF titers to determine if the trends seen here are generalizable. Most CM patients develop a detectable IgG titer to CF a few weeks into infection, and this titer generally decreases over time2,42,43. The samples utilized here were originally collected for a study focused on long lived cellular immunity and thus have a wide range of days from diagnosis to collection. Having such a broad sampling range of time from diagnosis may have negatively impacted the sensitivity of our assay, with the relative abundance of Coccidioides spp. specific antibodies waning over time. Interestingly, however, we were able to detect IgG antibody reactivity to the 31 peptides of interest in samples collected years after diagnosis (FIG. 13) indicating the presence of long-lived antibodies in response to Coccidioides spp. infection.

TABLE 9
CM patient metadata

| | | | | | Days from | Days from | | |
|---|---|---|---|---|---|---|---|---|
| | | | | | diagnosis to | CF titer to | | |
| Donor | | | CF | Year of | blood | blood | | |
| ID | Race | Ethnicity | titer | diagnosis | sample | sample | Diagnosis | Shipment |
| CM-1 | White | Not Hispanic | <1:2 | 2019 | 848 | 52 | pulmonary | Shipment_1 |
| CM-2 | White | Not Hispanic | <1:2 | 2019 | 1177 | 57 | pulmonary | Shipment_1 |
| CM-3 | Unknown | Unknown | 1:32 | 2020 | 665 | 7 | pulmonary | Shipment_1 |
| CM-4 | White | Hispanic | 1:128 | 2020 | 582 | 210 | pulmonary | Shipment_1 |
| CM-5 | White | Not Hispanic | 1:02 | 2013 | 3388 | 182 | pulmonary | Shipment_1 |
| CM-6 | Asian | Not Hispanic | 1:02 | 2018 | 1425 | 517 | pulmonary | Shipment_1 |
| CM-7 | White | Hispanic | <1:2 | 2021 | 155 | 154 | pulmonary | Shipment_1 |
| CM-8 | White | Hispanic | 1:08 | 2018 | 1604 | 90 | pulmonary | Shipment_1 |
| CM-9 | Asian | Not Hispanic | 1:08 | 2021 | 489 | 34 | pulmonary | Shipment_1 |
| CM-10 | White | Not Hispanic | not | 2022 | 13 | NA | pulmonary | Shipment_1 |
| | | | available | | | | | |
| CM-11 | Native American/ | Not Hispanic | not | 2020 | 891 | NA | pulmonary | Shipment_1 |
| | Alaska Native | | available | | | | | |
| CM-12 | White | Not Hispanic | 1:08 | 2020 | 635 | 635 | pulmonary | Shipment_1 |
| CM-13 | White | Not Hispanic | 1:256 | 2020 | 651 | 208 | pulmonary | Shipment_1 |
| CM-14 | Native American/ | Not Hispanic | 1:32 | 2022 | 133 | 133 | pulmonary | Shipment_1 |
| | Alaska Native | | | | | | | |
| CM-15 | White | Not Hispanic | 1:04 | 2020 | 758 | 665 | pulmonary | Shipment_1 |
| CM-16 | Black/African | Not Hispanic | <1:2 | 2022 | 118 | 118 | pulmonary | Shipment_1 |
| | American | | | | | | | |
| CM-17 | White | Not Hispanic | <1:2 | 2021 | 696 | 601 | pulmonary | Shipment_1 |
| CM-18 | White | Not Hispanic | <1:2 | 2021 | 90 | | not specified | Shipment_2 |
| CM-19 | White | Hispanic | <1:2 | 2022 | 370 | | not specified | Shipment_2 |
| CM-20 | White | Not Hispanic | 1:08 | 2016 | 2555 | | not specified | Shipment_2 |
| CM-21 | White | Hispanic | 1:16 | 2022 | 234 | | pulmonary | Shipment_2 |
| CM-22 | White | Not Hispanic | <1:2 | 2021 | 125 | | pulmonary | Shipment_2 |
| CM-23 | White | Not Hispanic | indeterminate | 2022 | 270 | | pulmonary | Shipment_2 |
| CM-24 | White | Hispanic | 1:16 | 2022 | 300 | | pulmonary | Shipment_2 |
| CM-25 | African American | Not Hispanic | 1:128 | 2023 | 5 | | pulmonary | Shipment_2 |
| CM-26 | White | Hispanic | positive | 2018 | 30 | | pulmonary | Shipment_2 |
| CM-27 | White | Not Hispanic | positive | 2023 | 28 | | pulmonary | Shipment_2 |
| CM-28 | White | Not Hispanic | not | NA | NA | NA | not specified | Shipment_2 |
| | | | available | | | | | |
| CM-29 | White | Not Hispanic | not | NA | NA | NA | not specified | Shipment_2 |
| | | | available | | | | | |
| CM-30 | White | Hispanic | 1:16 | 2022 | 326 | | pulmonary | Shipment_2 |
| CM-31 | Asian | Not Hispanic | 1:02 | 2023 | 81 | | pulmonary | Shipment_2 |
| CM-32 | 2 or more races | Declined | 1:08 | 2022 | 1 | | pulmonary | Shipment_2 |
| CM-33 | White | Unknown | 1:04 | 2023 | 55 | | pulmonary | Shipment_2 |
| CM-34 | White | Not Hispanic | 1:16 | 2023 | 7 | | pulmonary | Shipment_2 |
| CM-35 | White | Hispanic | 1:02 | 2023 | 81 | | pulmonary | Shipment_2 |
| CM-36 | White | Not Hispanic | 1:02 | 2023 | 40 | | disseminated | Shipment_2 |
| CM-37 | White | Not Hispanic | 1:16 | 2023 | 88 | | pulmonary | Shipment_2 |
| CM-38 | White | Hispanic | <1:2 | 2023 | 38 | | pulmonary | Shipment_2 |
| CM-39 | White | Not Hispanic | <1:2 | 2020 | 72 | | pulmonary | Shipment_2 |
| CM-40 | White | Not Hispanic | positive | 2023 | 12 | | pulmonary | Shipment_2 |
| CM-41 | African American | Not Hispanic | <1:2 | 2023 | 5 | | pulmonary | Shipment_2 |
| CM-42 | White | Hispanic | 1:32 | 2023 | 66 | | disseminated | Shipment_2 |
| CM-43 | White | Hispanic | 1:04 | 2023 | 70 | | pulmonary | Shipment_2 |
| CM-44 | White | Not Hispanic | 1:04 | 2021 | 943 | | pulmonary | Shipment_2 |
| CM-45 | White | Not Hispanic | not | NA | NA | NA | not specified | Shipment_2 |
| | | | available | | | | | |
| CM-46 | | | >1024 | 2013 | 0 | 180 | disseminated | Shipment_3 |
| CM-47 | | | not | 2013 | NA | NA | asymptomatic | Shipment_3 |
| | | | available | | | | | |
| CM-48 | | | 1:02 | 2013 | 0 | 16 | pulmonary | Shipment_3 |
| CM-49 | | | not | 2013 | NA | NA | pulmonary | Shipment_3 |
| | | | available | | | | | |
| CM-50 | | | not | 2013 | NA | NA | pulmonary | Shipment_3 |
| | | | available | | | | | |
| CM-51 | | | <1:2 | 2013 | 139 | 5 | pulmonary | Shipment_3 |
| CM-52 | | | >1024 | 2014 | 0 | 0 | pulmonary | Shipment_3 |
| CM-53 | | | 1:256 | 2014 | 0 | 3 | disseminated | Shipment_3 |
| CM-54 | | | 1:64 | 2014 | 0 | 7 | pulmonary | Shipment_3 |
| CM-55 | | | 1:04 | 2014 | 17 | −12 | pulmonary | Shipment_3 |
| CM-56 | | | not | 2002 | NA | NA | disseminated | Shipment_3 |
| | | | available | | | | | |
| CM-57 | | | 1:16 | 2014 | 0 | 9 | pulmonary | Shipment_3 |
| CM-58 | | | 1:08 | 2015 | 0 | 0 | pulmonary | Shipment_3 |
| CM-59 | | | not | 2015 | NA | NA | pulmonary | Shipment_3 |
| | | | available | | | | | |
| CM-60 | | | not | 2015 | NA | NA | pulmonary | Shipment_3 |
| | | | available | | | | | |
| CM-61 | | | not | 2015 | NA | NA | pulmonary | Shipment_3 |
| | | | available | | | | | |
| CM-62 | | | not | 2011 | NA | NA | pulmonary | Shipment_3 |
| | | | available | | | | | |
| CM-63 | | | not | 2011 | NA | NA | pulmonary | Shipment_3 |
| | | | available | | | | | |
| CM-64 | | | not | 2011 | NA | NA | pulmonary | Shipment_3 |
| | | | available | | | | | |
| CM-65 | | | not | 2013 | NA | NA | pulmonary | Shipment_3 |
| | | | available | | | | | |
| CM-66 | | | not | 2013 | NA | NA | pulmonary | Shipment_3 |
| | | | available | | | | | |
| CM-67 | | | not | 2013 | NA | NA | pulmonary | Shipment_3 |
| | | | available | | | | | |
| CM-68 | | | not | 2014 | NA | NA | pulmonary | Shipment_3 |
| | | | available | | | | | |
| CM-69 | | | not | NA | NA | NA | pulmonary | Shipment_3 |
| | | | available | | | | | |
| CM-70 | | | not | NA | NA | NA | pulmonary | Shipment_3 |
| | | | available | | | | | |

53% male, 47% female
Age 23-87, average 55.5 years

Although our sample size is modest, the statistical approach utilized here is robust with zero peptides being detected as significantly enriched in the IgG non-endemic control cohort when running a flipped analysis. Furthermore, our random permutation analysis of the human IgG data indicated that our false discovery rate was quite low. It is likely that there are more reactive peptides and proteins of interest that we are not powered to identify as significant with our current cohort sizes. Another limitation to consider is that the control cohorts in this study were obtained from a blood bank in Louisiana, and we have no metadata for these samples other than where the collection was made. While these samples were taken from outside the endemic CM region, it is possible that some of them previously lived in or visited a Coccidioides spp. endemic region where they were exposed to the fungus. This is one possible explanation for some of the reactivity we see in the control cohort samples in FIG. 4A. However, using a commercial immunodiffusion kit, we did not find evidence of antibody reactivity against Coccidioides spp, or the other endemic fungi on the panel. Of note we also only observed two out of three confirmed CM patients show reactivity in the commercial immunodiffusion assay. The CM patient that tested negative was positive by immunodiffusion at the UC Davis lab and negative for CF, highlighting differences in results between labs and tests in CM.

We observed significant IgG reactivity to the spherule outer wall glycoprotein (SOWgp), a well characterized immunoreactive protein. This protein has previously been shown to be recognized by the cellular and humoral immune system44. SOWgp is found on the cell walls of spherules and endospores, and is expressed primarily during the parasitic phase within the host45. SOWgp abundances fluctuates, as it is concentrated during cell maturation and later degraded by Mep1 to prevent immune system recognition and phagocytosis⁴⁵. It has been suggested that high titers of antibodies against SOWgp contribute to fungal cell opsonization and phagocytosis^{46,47}. Additionally, reactivity to SOWgp has been observed in skin test positive patients, although it has not been developed into a diagnostic^{46,47}. Recombinant SOWgp as a subcutaneous vaccine aided in fungal clearance in mice and production of IFN-γ⁴⁷. Before our study, the epitope regions associated with antibody binding to SOWgp were not defined. Here, we highlight two linear SOWgp regions commonly targeted by antibodies in patients with CM. We identified four significantly reactive peptides for SOWgp (CWP_085538, CWP_085539, CWP_103986, CWP_103987), three of which overlap by eight AAs, and likely contain a single epitope, “YCDTYGCD” (SEQ ID NO: 51). The fourth peptide (CWP_085539) is designed from a different region of SOWgp and represents a second reactive epitope. These peptide sequences had very low or no sequence similarity in our BLASTP results, supporting their utility in a diagnostic for CM.

The other peptides come from uncharacterized proteins, several of which appear to be orthologs (see, Table 4). We used predictive algorithms to give insight into the biological role of these novel antigens. Six of these proteins are predicted to be secreted from the cell, which support an interaction between the proteins and the immune system. The other eight proteins are localized within the cell. Additionally, transcriptional evidence suggests the majority of the proteins have increased transcriptional abundance during the host specific lifecycle.

Out of the 31 IgG peptides of interest, 27 come from five uncharacterized proteins. We used predictive algorithms to give insight into the biological roles of these novel antigens. Four out of six of the identified proteins are predicted to be extracellular, which supports an interaction between the proteins and the immune system. We searched FungiDB to gain insight into these proteins and found limited information or publications beyond those focused on transcript regulation30,36. Interestingly, the protein for which we detected the largest number of reactive peptides was an uncharacterized protein, IgG antigen 1, which is highly specific to Coccidioides spp. based on our BLASTP search. Seven of the significantly reactive peptides in IgG antigen 1 were designed from amino acid positions 201-385, a region of the protein that is highly repetitive and proline rich. These peptides all include the same putative epitope, “PESKPFDPY” (SEQ ID NO: 52), where the bold letters indicate AAs conserved across all seven peptides, and the non-bold AAs are the consensus AAs (FIG. 14). Furthermore, two of the peptides contain two copies of this epitope (CWP_028597, CWP_054481), and these two peptides have the highest median Z score in the CM samples of any of the identified peptides of interest. Apart from this repeated epitope, IgG antigen 1 also has multiple other immunoreactive epitopes (FIG. 4A, B), clearly indicating that it is an immunodominant protein.

Although the biological function of these antigenic proteins is undefined, a major goal of this study was to identify novel diagnostic targets. Therefore, a subset of highly reactive peptides were commercially synthesized and used to build an ELISA. The selection was based on preliminary data analysis and includes nine different proteins (including all six significant proteins of interest), with one to four peptide sequences per protein (Table 6). We tested the ability of the ELISA assay to discriminate between CM patients and healthy blood bank controls who had not previously been assayed with our CWP PepSeq library (Validation cohort), and thus did not inform peptide selection. Using this independent sample set, we demonstrated the peptides identified in this study could successfully discriminate between disease and non-disease at high accuracy (ROC 0.95). These data support that binding signals to these linear epitopes in an ELISA can differentiate between CM positive and negative donors with sensitivity (89%) and specificity (90%) that is competitive with current clinical diagnostics16. Future studies with full length proteins will be needed to determine the potential impact of conformational specific epitopes in the proteins of interest identified here on the performance of the ELISA assay.

Lastly, we report here the first analysis of IgG antibody reactivity against Coccidioides spp. in naturally infected PTMs. We detected significantly higher reactivity in our animals with CM to two peptides designed from the IgG antigen 3 protein that was also identified in our human IgG PepSeq screen (described above). PTM are being used in Coccidioides spp. vaccine trials and a better understanding of the B cell response to natural infection in these animals may be valuable in these efforts (as presented at Coccodioidomycosis Study Group 2024-25). While our sample size here is limited, our results show that PepSeq can be used to detect antibody reactivity in PTMs and that B cell responses to infection in humans and PTMs target some of the same proteins/epitopes (FIG. 8D). Future studies will be needed to further map the B cell responses in PTMs in response to natural infection with Coccidioides spp.

The epitope regions identified herein may be used in peptide-based diagnostics, development of subunit vaccines, and development of therapeutic binders, including monoclonal antibodies. Some embodiments of the invention may comprise the administration of a pharmaceutical composition to the subject that has been previously diagnosed with a fungal infection, such as Coccidioides infection. For example, in some embodiments, the subject may have been previously diagnosed with Coccidioidomycosis by one skilled in the art (e.g., a physician or a veterinarian) such that a therapeutic treatment is warranted by the diagnosis. Moreover, in other embodiments, the invention may comprise the administration of a pharmaceutical composition to a subject that may have not yet been exposed to a Coccidioides-infected environment or individual. As such, the administration of the pharmaceutical composition may function as a vaccine or prophylactic agent to limit any potential fungal infection that could occur, or to improve the subject's immune response to the fungi.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

### REFERENCES

1. Bays, D. J. & Thompson, G. R., 3rd. Coccidioidomycosis. Infect. Dis. Clin. North Am. 35, 453-469 (2021).
2. Gabe, L. M., Malo, J. & Knox, K. S. Diagnosis and Management of Coccidioidomycosis. Clin. Chest Med. 38, 417-433 (2017).
3. Galgiani, J. N. et al. 2016 Infectious Diseases Society of America (IDSA) Clinical Practice Guideline for the Treatment of Coccidioidomycosis. Clin. Infect. Dis. 63, e112-46 (2016).
4. Donovan, F. M. et al. Delays in coccidioidomycosis diagnosis and associated healthcare utilization, Tucson, Arizona, USA. Emerg. Infect. Dis. 25, 1745-1747 (2019).
5. McHardy, I. H., Barker, B. & Thompson, G. R., 3rd. Review of Clinical and Laboratory Diagnostics for Coccidioidomycosis. J. Clin. Microbiol. 61, e0158122 (2023).
6. Hung, C. Y., Yu, J. J., Lehmann, P. F. & Cole, G. T. Cloning and expression of the gene which encodes a tube precipitin antigen and wall-associated beta-glucosidase of Coccidioides immitis. Infect. Immun. 69, 2211-2222 (2001).
7. Johnson, S. M. & Pappagianis, D. The coccidioidal complement fixation and immunodiffusion-complement fixation antigen is a chitinase. Infect. Immun. 60, 2588-2592 (1992).
8. Durkin, M. et al. Diagnosis of coccidioidomycosis with use of the Coccidioides antigen enzyme immunoassay. Clin. Infect. Dis. 47, e69-73 (2008).
9. Kassis, C. et al. Diagnosis of Coccidioidomycosis with the Second-Generation Miravista IgG and IgM Enzyme Immunoassay and the Role of Adding Miravista Coccidioides Antigen Detection to Immunodiagnostic Assays. Med. Mycol. 62, (2024).
10. Grill, F. J. et al. Clinical Laboratory Utility of a Humanized Antibody in Commercially Available Enzyme Immunoassays for Coccidioidomycosis. Microbiol Spectr e0257322 (2022) doi: 10.1128/spectrum.02573-22.
11. Grill, F. J. et al. Development of a rapid lateral flow assay for detection of anti-coccidioidal antibodies. J. Clin. Microbiol. 61, e0063123 (2023).
12. Pappagianis, D. & Zimmer, B. L. Serology of coccidioidomycosis. Clin. Microbiol. Rev. 3, 247-268 (1990).
13. Khan, S. et al. Interlaboratory agreement of coccidioidomycosis enzyme immunoassay from two different manufacturers. Med. Mycol. 57, 441-446 (2019).
14. Donovan, F. M. et al. Comparison of a Novel Rapid Lateral Flow Assay to Enzyme Immunoassay Results for Early Diagnosis of Coccidioidomycosis. Clin. Infect. Dis. 73, e2746-e2753 (2021).
15. Stevens, D. A. et al. Comparative study of newer and established methods of diagnosing coccidioidal meningitis. J. Fungi (Basel) 6, 125 (2020).
16. Malo, J. et al. Comparison of three anti-Coccidioides antibody enzyme immunoassay kits for the diagnosis of coccidioidomycosis. Med. Mycol. 58, 774-778 (2020).
17. Barker, B. M., Jewell, K. A., Kroken, S. & Orbach, M. J. The population biology of Coccidioides: epidemiologic implications for disease outbreaks. Ann. N. Y. Acad. Sci. 1111, 147-163 (2007).
18. Neafsey, D. E. et al. Population genomic sequencing of Coccidioides fungi reveals recent hybridization and transposon control. Genome Res. 20, 938-946 (2010).
19. Lequin, R. M. Enzyme immunoassay (EIA)/enzyme-linked immunosorbent assay (ELISA). Clin Chem 51, 2415-2418 (2005).
20. Henson, S. N. et al. PepSeq: a fully in vitro platform for highly multiplexed serology using customizable DNA-barcoded peptide libraries. Nat. Protoc. (2022) doi: 10.1038/s41596-022-00766-8.
21. Piña, A. et al. Mapping disparities in viral infection rates using highly multiplexed serology. mSphere 9, e0012724 (2024).
22. Elko, E. A. et al. Recurrent SARS-COV-2 mutations at Spike D796 evade antibodies from pre-Omicron convalescent and vaccinated subjects. Microbiol Spectr e0329123 (2024) doi: 10.1128/spectrum.03291-23.
23. Elko, E. A. et al. COVID-19 vaccination elicits an evolving, cross-reactive antibody response to epitopes conserved with endemic coronavirus spike proteins. Cell Rep. 40, 111022 (2022).
24. Kelley, E. J. et al. Virome-wide detection of natural infection events and the associated antibody dynamics using longitudinal highly-multiplexed serology. Nat. Commun. 14, 1783 (2023).
25. Ladner, J. T. et al. Epitope-resolved profiling of the SARS-COV-2 antibody response identifies cross-reactivity with endemic human coronaviruses. Cell Rep Med 2, 100189 (2021).
26. Elko, E. A. et al. PepSeq as a highly multiplexed platform for melioidosis antigen discovery and vaccine development. Front. Immunol. 16, (2025).
27. Mitchell, N. M. et al. Proteogenomic re-annotation of Coccidioides posadasii strain Silveira. Proteomics 18, (2018).
28. Edgar, R. C. Search and clustering orders of magnitude faster than BLAST. Bioinformatics 26, 2460-2461 (2010).
29. Thumuluri, V., Almagro Armenteros, J. J., Johansen, A. R., Nielsen, H. & Winther, O. DeepLoc 2.0: multi-label subcellular localization prediction using protein language models. Nucleic Acids Res. 50, W228-W234 (2022).
30. Basenko, E. Y. et al. FungiDB: An Integrated Bioinformatic Resource for Fungi and Oomycetes. J Fungi (Basel) 4, (2018).
31. Sayers, E. W. et al. Database resources of the National Center for Biotechnology Information in 2025. Nucleic Acids Res. 53, D20-D29 (2025).
32. Seabold, S. & Perktold, J. Statsmodels: Econometric and statistical modeling with python. in Proceedings of the 9th Python in Science Conference (SciPy, 2010). doi: 10.25080/majora-92bf1922-011.
33. Sievers, F. et al. Fast, scalable generation of high-quality protein multiple sequence alignments using Clustal Omega. Mol. Syst. Biol. 7, 539 (2011).
34. Guerriero, K. A. et al. Recrudescence of natural coccidioidomycosis during combination antiretroviral therapy in a pigtail macaque experimentally infected with simian immunodeficiency virus. AIDS Res. Hum. Retroviruses 37, 505-509 (2021).
35. Koistinen, K. et al. Coccidioidomycosis in nonhuman primates: Pathologic and clinical findings. Vet. Pathol. 55, 905-915 (2018).
36. Whiston, E. et al. Comparative transcriptomics of the saprobic and parasitic growth phases in Coccidioides spp. PLOS One 7, e41034 (2012).
37. Richardson, J. P. & Moyes, D. L. Adaptive immune responses to Candida albicans infection. Virulence 6, 327-337 (2015).
38. Spellberg, B. et al. Antibody titer threshold predicts anti-candidal vaccine efficacy even though the mechanism of protection is induction of cell-mediated immunity. J. Infect. Dis. 197, 967-971 (2008).
39. Patel, A. & Jialal, I. Biochemistry, immunoglobulin A. in StatPearls (StatPearls Publishing, Treasure Island (FL), 2025).
40. McCotter, O. Z. et al. Update on the Epidemiology of coccidioidomycosis in the United States. Med. Mycol. 57, S30-S40 (2019).
41. Vidarsson, G., Dekkers, G. & Rispens, T. IgG subclasses and allotypes: from structure to effector functions. Front. Immunol. 5, 520 (2014).
42. McHardy, I. H. et al. Coccidioidomycosis Complement Fixation Titer Trends in the Age of Antifungals. J. Clin. Microbiol. 56, (2018).
43. Smith, C. E., Saito, M. T. & Simons, S. A. Pattern of 39,500 serologic tests in coccidioidomycosis. J. Am. Med. Assoc. 160, 546-552 (1956).
44. Hung, C. Y., Ampel, N. M., Christian, L., Seshan, K. R. & Cole, G. T. A major cell surface antigen of Coccidioides immitis which elicits both humoral and cellular immune responses. Infect. Immun. 68, 584-593 (2000).
45. Cole, G. T. et al. Isolation and morphology of an immunoreactive outer wall fraction produced by spherules of Coccidioides immitis. Infect. Immun. 56, 2686-2694 (1988).
46. Hung, C.-Y. et al. A metalloproteinase of Coccidioides posadasii contributes to evasion of host detection. Infect. Immun. 73, 6689-6703 (2005).
47. Cox, R. A. & Magee, D. M. Coccidioidomycosis: host response and vaccine development. Clin. Microbiol. Rev. 17, 804-39, table of contents (2004).

What is claimed:

1. A method of detecting in a sample the presence of an antibody that binds to a Coccidioides protein, the method comprising:
providing a biological sample from a subject suspected to be infected with Coccidioides;
contacting the biological sample with a peptide comprising an amino acid sequence having at least 85% sequence identity to an amino acid sequence selected from the group consisting of SEQ ID NO: 1-50; and
detecting antigen-antibody complexes formed.

2. The method of claim 1, wherein the peptide comprises an amino acid sequence having at least 85% sequence identity to an amino acid sequence selected from the group consisting of SEQ ID NO: 2-4, 8-9, 11-18, 23-25, 27, 29, 30-33, 35, 37-41, 43-44, and 48.

3. The method of claim 1, wherein the peptide comprises an amino acid sequence having at least 85% sequence identity to an amino acid sequence selected from the group consisting of SEQ ID NO: 1, 3, 7, 13, 15, 17, 24, 26, 29, 32, 36, 38, 39, 40, 43, and 44.

4. The method of claim 1, further comprising detecting in the sample the presence of one or more additional antibodies that each binds to a Coccidioides protein, wherein the sample is contacted with one or more additional peptides comprising an amino acid sequence having at least 85% sequence identity to an amino acid sequence selected from the group consisting of SEQ ID NO: 1-50.

5. The method of claim 4, wherein the one or more additional peptides comprise an amino acid sequence having at least 85% sequence identity to an amino acid sequence selected from the group consisting of SEQ ID NO: 2-4, 8-9, 11-18, 23-25, 27, 29, 30-33, 35, 37-41, 43-44, and 48.

6. The method of claim 4, wherein the one or more additional peptides comprise an amino acid sequence having at least 85% sequence identity to an amino acid sequence selected from the group consisting of SEQ ID NO: 1, 3, 7, 13, 15, 17, 24, 26, 29, 32, 36, 38, 39, 40, 43, and 44.

7. The method of claim 1, wherein the method comprises detecting in the sample the presence of a first, second, third, fourth, fifth, and/or sixth antibody that binds to a Coccidioides protein,
wherein the first peptide comprises an amino acid sequence having at least 85% sequence identity to an amino acid sequence selected from the group consisting of SEQ ID NO: 38, 39, 43, and 44,
wherein the second peptide comprises an amino acid sequence having at least 85% sequence identity to an amino acid sequence selected from the group consisting of SEQ ID NO: 8-9, 11-18, 23-25, 27, and 29-33,
wherein the third peptide comprises an amino acid sequence having at least 85% sequence identity to an amino acid sequence selected from the group consisting of SEQ ID NO: 2-4 and 35,
wherein the fourth peptide comprises an amino acid sequence having at least 85% sequence identity to an amino acid sequence selected from the group consisting of SEQ ID NO: 40-41,
wherein the fifth peptide comprises an amino acid sequence having at least 85% sequence identity to an amino acid sequence consisting of SEQ ID NO: 48, and/or
wherein the sixth peptide comprises an amino acid sequence having at least 85% sequence identity to an amino acid sequence consisting of SEQ ID NO: 37.

8. The method of claim 1, wherein the biological sample is whole blood, serum, or plasma.

9. The method of claim 8, wherein the antibody is IgG.

10. The method of claim 8, wherein the antibody is IgA or IgM.

11. The method of claim 1, wherein the subject is a human.

12. The method of claim 1, wherein the method of detecting antigen-antibody complexes comprises a technique selected from the group consisting of enzyme-linked immunosorbent assay (ELISA), western blot, flow cytometry, and immunoaffinity chromatography.

13. The method of claim 1, wherein detection of the antigen-antibody complex is indicative of a Coccidioides infection.

14. The method of claim 13, further comprising treating the subject for a Coccidioides infection if an antigen-antibody complex is detected.

15. An immunogenic composition, comprising:
one or more peptides comprising an amino acid sequence having at least 85% sequence identity to an amino acid sequence selected from the group consisting of SEQ ID NO: 1-50; and
a pharmaceutically acceptable carrier or adjuvant.

16. The immunogenic composition of claim 15, wherein the amino acid sequence comprises 85% sequence identity to an amino acid sequence selected from the group consisting of SEQ ID NO: 2-4, 8-9, 11-18, 23-25, 27, 29, 30-33, 35, 37-41, 43-44, and 48.

17. The immunogenic composition of claim 15, wherein the amino acid sequence comprises 85% sequence identity to an amino acid sequence selected from the group consisting of SEQ ID NO: 1, 3, 7, 13, 15, 17, 24, 26, 29, 32, 36, 38, 39, 40, 43, and 44.

18. The immunogenic composition of claim 15, wherein the composition comprises a first, second, third, fourth, fifth, and/or sixth peptide,
wherein the first peptide comprises an amino acid sequence having at least 85% sequence identity to an amino acid sequence selected from the group consisting of SEQ ID NO: 38, 39, 43, and 44,
wherein the second peptide comprises an amino acid sequence having at least 85% sequence identity to an amino acid sequence selected from the group consisting of SEQ ID NO: 8-9, 11-18, 23-25, 27, and 29-33,
wherein the third peptide comprises an amino acid sequence having at least 85% sequence identity to an amino acid sequence selected from the group consisting of SEQ ID NO: 2-4 and 35,
wherein the fourth peptide comprises an amino acid sequence having at least 85% sequence identity to an amino acid sequence selected from the group consisting of SEQ ID NO: 40-41,
wherein the fifth peptide comprises an amino acid sequence having at least 85% sequence identity to an amino acid sequence consisting of SEQ ID NO: 48, and/or
wherein the sixth peptide comprises an amino acid sequence having at least 85% sequence identity to an amino acid sequence consisting of SEQ ID NO: 37.

19. A method of eliciting an immune response in a subject, the method comprising administering to the subject the immunogenic composition of claim 15.

20. A kit for detecting the presence of Coccidioides in a subject comprising one or more peptides comprising an amino acid sequence having at least 85% sequence identity to an amino acid sequence selected from the group consisting of SEQ ID NOS: 1-50; and optionally, one or more of the following: (i) the peptides comprise an amino acid sequence having at least 85% sequence identity to an amino acid sequence selected from the group consisting of SEQ ID NO: 2-4, 8-9, 11-18, 23-25, 27, 29, 30-33, 35, 37-41, 43-44, and 48; (ii) the kit comprises a first, second, third, fourth, fifth, and/or sixth peptide, wherein the first peptide comprises an amino acid sequence having at least 85% sequence identity to an amino acid sequence selected from the group consisting of SEQ ID NO: 38, 39, 43, and 44, wherein the second peptide comprises an amino acid sequence having at least 85% sequence identity to an amino acid sequence selected from the group consisting of SEQ ID NO: 8-9, 11-18, 23-25, 27, and 29-33, wherein the third peptide comprises an amino acid sequence having at least 85% sequence identity to an amino acid sequence selected from the group consisting of SEQ ID NO: 2-4 and 35, wherein the fourth peptide comprises an amino acid sequence having at least 85% sequence identity to an amino acid sequence selected from the group consisting of SEQ ID NO: 40-41, wherein the fifth peptide comprises an amino acid sequence having at least 85% sequence identity to an amino acid sequence consisting of SEQ ID NO: 48, and/or wherein the sixth peptide comprises an amino acid sequence having at least 85% sequence identity to an amino acid sequence consisting of SEQ ID NO: 37; (iii) wherein the one or more peptides comprise an amino acid sequence having at least 85% sequence identity to an amino acid sequence selected from the group consisting of SEQ ID NO: 1, 3, 7, 13, 15, 17, 24, 26, 29, 32, 36, 38, 39, 40, 43, and 44; (iv) wherein the kit comprises 16 peptides comprising an amino acid sequence having at least 85% sequence identity to an amino acid sequence as set forth by SEQ ID NO: 1, 3, 7, 13, 15, 17, 24, 26, 29, 32, 36, 38, 39, 40, 43, and 44; (v) where each peptide is biotinylated; and/or (vi) wherein the kit comprises a plate having microwells coated with the peptides.
